(19) United States
(12) Patent Application Publication
(10) Pub. No.: US 2022/0387572 A1
(43) Pub. Date: Dec. 8, 2022

(54) RENAL CELL CARCINOMA (RCC) THERAPY USING GENETICALLY ENGINEERED T CELLS TARGETING CD70

(71) Applicant: CRISPR THERAPEUTICS AG, Zug (CH)

(72) Inventors: Jonathan Alexander TERRETT, Cambridge, MA (US); Mary-Lee DEQUÉANT, Cambridge, MA (US); Matthias WILL, Cambridge, MA (US)

(73) Assignee: CRISPR THERAPEUTICS AG, Zug (CH)

(21) Appl. No.: 17/776,860

(22) PCT Filed: Nov. 13, 2020
(86) PCT No.: PCT/IB2020/060719

§ 371 Date: May 13, 2022

Related U.S. Application Data
(60) Provisional application No. 63/034,552, filed on Jun. 4, 2020.
(60) Provisional application No. 62/934,961, filed on Nov. 13, 2019.

(51) Int. Cl. A61K 39/00; A61K 31/7076; A61K 31/675

(52) U.S. Cl. CPC A61K 39/001138; A61K 31/7076; A61K 31/675; A61K 2039/5156; A61K 2039/5158; A61K 2039/868; C07K 2319/03; C07K 14/7051

(57) ABSTRACT

Aspects of the present disclosure relate to compositions comprising a population of genetically engineered T cells that expresses a chimeric antigen receptor (CAR) that binds CD70, and methods of using such for the treatment of renal cell cancer (RCC).

### CROSS-REFERENCE TO RELATED APPLICATIONS

This application claims the benefit of priority to U.S. Provisional Patent Application No. 62/934,961, filed Nov. 13, 2019, and U.S. Provisional Patent Application No. 63/034,552, filed Jun. 4, 2020. Each of the prior applications is hereby incorporated by reference in its entirety.

### BACKGROUND

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

### SUMMARY

The present disclosure is based, at least in part, on the surprising discovery that anti-CD70 CAR+ T cells reduced tumor burden in various subcutaneous renal cell carcinoma (RCC) xenograft models. It has also been demonstrated that the anti-CD70 CAR T cells described herein displayed long-term in vivo efficacy that prevented tumor growth after re-exposure to tumor cells. Significant reductions in tumor burden were also observed after redosing of anti-CD70 CAR T cells. Further, CTX130 cell distribution, expansion, and persistence were observed in human subjects receiving the CAR-T cells. Superior treatment efficacy was also observed in human RCC patients who received the CTX130 cell treatment.

Accordingly, aspects of the present disclosure provide methods for treating renal cell carcinoma (RCC) comprising (i) subjecting a human patient having RCC to lymphodepletion treatment, and (ii) administering to the human patient a population of genetically engineered T cells (also referred to as CAR T cell therapy) after step (i).

Some aspects of the present disclosure provide a method for treating renal cell carcinoma (RCC), the method comprising (i) subjecting a human patient having RCC to a first lymphodepletion treatment; and (ii) administering to the human patient a first dose of a population of genetically engineered T cells after step (i), wherein the population of genetically engineered T cells comprises T cells expressing a chimeric antigen receptor (CAR) that binds CD70, and comprising a disrupted β2M gene, a disrupted CD70 gene, and a disrupted TRAC gene, into which a nucleotide sequence encoding the CAR is inserted. In some examples, the population of genetically engineered T cells are CTX130 cells as disclosed herein.

In some embodiments, the first lymphodepletion treatment in step (i) comprises co-administering to the human patient fludarabine at 30 mg/m² and cyclophosphamide at 500 mg/m² intravenously per day for three days.

In some embodiments, prior to step (i), the human patient does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, and (f) grade ≥2 acute neurological toxicity.

In some embodiments, step (i) is performed about 2-7 days prior to step (ii). Alternatively or in addition, step (ii) is performed by administering the population of genetically engineered T cells to the human patient intravenously at the first dose, which may be about 1×10⁶ CAR+ cells to about 1×10⁹ CAR+ cells. In some examples, the first dose may range from about 3×10⁷ to about 9×10⁸ CAR+ cells.

In some embodiments, prior to step (ii) and after step (i), the human patient does not show one or more of the following features: (a) active uncontrolled infection, (b) worsening of clinical status compared to the clinical status prior to step (i), and (c) grade ≥2 acute neurological toxicity.

In some embodiments, methods further comprise (iii) monitoring the human patient for development of acute toxicity after step (ii). In some embodiments, acute toxicity comprises cytokine release syndrome (CRS), neurotoxicity (e.g., ICANS), tumor lysis syndrome, GvHD, on target off-tumor toxicity, and/or uncontrolled T cell proliferation. The on target off-tumor toxicity may comprises activity of the population of genetically engineered T cells against activated T lymphocytes, B lymphocytes, dendritic cells, osteoblasts and/or renal tubular-like epithelium.

In some embodiments, methods further comprise (iv) subjecting the human patient to a second lymphodepletion treatment, and (v) administering to the human patient a second dose of the population of genetically engineered T cells after step (ii). In some examples, the human patient does not show one or more of the following after step (ii): (a) dose-limiting toxicity (DLT), (b) grade 4 CRS that does not resolve to grade 2 within 72 hours, (c) grade >1 GvHD, (d) grade ≥3 neurotoxicity, (e) active infection, (f) hemodynamically unstable, and (g) organ dysfunction. The second dose of the population of genetically engineered T cells may be administered to the subject about 8 weeks to about 2 years after the first dose. In some instances, the second dose may be administered to the subject about 8-10 weeks after the first dose. In other instances, the second dose may be administered to the subject about 14-18 weeks after the first dose.

In some embodiments, the second lymphodepletion treatment in step (iv) comprises co-administering to the human patient fludarabine at 30 mg/m² and cyclophosphamide at 500 mg/m² intravenously per day for 1-3 days.

In some embodiments, step (v) is performed 2-7 days after step (iv). In some embodiments, step (v) is performed by administering the population of genetically engineered T cells to the human patient intravenously at the second dose, which can be about 1×10⁶ CAR+ cells to about 1×10⁹ CAR+ cells. In some examples, the second dose may range from about 3×10⁷ to about 9×10⁸ CAR+ cells.

In some embodiments the method may further comprise (vi) subjecting the human patient to a third lymphodepletion treatment, and (vii) administering to the human patient a third dose of the population of genetically engineered T cells about 8 weeks to about 2 years (e.g., about 14-18 weeks) after step (ii). In some instances, the second dose of the population of genetically engineered T cells is administered about 8 weeks to about two years (e.g., about 8-10 weeks) after step (ii). Alternatively or in addition, the third dose of the population of genetically engineered T cells may be administered to the subject about 8 weeks to about 2 years after the second dose. In some instances, the third dose may be administered to the subject about 8-10 weeks after the second dose. In other instances, the third dose may be administered to the subject about 14-18 weeks after the second dose.

In some instances, the human patient does not show one or more of the following after step (v): (a) dose-limiting toxicity (DLT), (b) grade 4 CRS that does not resolve to grade 2 within 72 hours, (c) grade >1 GvHD, (d) grade ≥3 neurotoxicity, (e) active infection, (f) hemodynamically unstable, and (g) organ dysfunction.

In some embodiments, the third lymphodepletion treatment in step (vi) comprises co-administering to the human patient fludarabine at 30 mg/m² and cyclophosphamide at 500 mg/m² intravenously per day for 1-3 days.

In some embodiments, step (vii) is performed 2-7 days after step (vi). In some embodiments, step (vii) is performed by administering the population of genetically engineered T cells to the human patient intravenously at the third dose, which is about 1×10⁶ CAR+ cells to about 1×10⁹ CAR+ cells. For example, the third dose may range from about 3×10⁷ to about 9×10⁸ CAR+ cells.

In some embodiments, the first dose, the second dose, and/or the third dose of the population of genetically engineered T cells is 1×10⁶ CAR+ cells, 3×10⁷ CAR+ cells, 1×10⁸ CAR+ cells, or 1×10⁹ CAR+ cells. In some examples, the first dose, the second dose, and/or the third dose of the population of genetically engineered T cells is about 1.5×10⁸ CAR⁺ cells. In some examples, the first dose, the second dose, and/or the third dose of the population of genetically engineered T cells is about 3×10⁸ CAR⁺ cells. In some examples, the first dose, the second dose, and/or the third dose of the population of genetically engineered T cells is about 4.5×10⁸ CAR⁺ cells. In some examples, the first dose, the second dose, and/or the third dose of the population of genetically engineered T cells is about 6×10⁸ CAR⁺ cells. In some examples, the first dose, the second dose, and/or the third dose of the population of genetically engineered T cells is about 7.5×10⁸ CAR⁺ cells. In some examples, the first dose, the second dose, and/or the third dose of the population of genetically engineered T cells is about 9×10⁸ CAR⁺ cells. In some examples, the first dose, the second dose, and/or the third dose of the population of genetically engineered T cells is about 1×10⁹ CAR⁺ cells. In some examples, the first dose of the population of genetically engineered T cells is the same as the second and/or third dose of the population of genetically engineered T cells. In other examples, the first dose of the population of genetically engineered T cells is lower than the second and/or third dose of the population of genetically engineered T cells.

In some embodiments, the human patient shows stable disease or disease progress. In other embodiments, the human patient has unresectable or metastatic RCC. In yet other embodiments, the human patient has relapsed or refractory RCC. In some embodiments, the human patient has clear cell differentiation (e.g., predominantly). In some embodiments, the human patient has undergone a prior anti-cancer therapy. In some embodiments, the prior anti-cancer therapy comprises a checkpoint inhibitor, a tyrosine kinase inhibitor, a vascular endothelial factor (VEGF) inhibitor, or a combination thereof. In some embodiments, the human patient is subject to an anti-cytokine therapy. In some embodiments, the human patient is subject to an additional anti-cancer therapy after treatment with the population of genetically engineered T cells.

In some embodiments, the human patient has one or more of the following features: (a) Karnofsky performance status (KPS) ≥80%, and (b) adequate organ function, (c) free of treatment with prior anti-CD70 or adoptive T cell or NK cell therapy, (d) free of prior anaphylactic reaction to lymphodepletion therapy, (e) free of brain metastases, (f) free of prior central nervous system disorders, (g) free of unstable angina, arrhythmia, and/or myocardial infarction, (h) free of diabetes mellitus, (i) free of uncontrolled infections, (j) free of immunodeficiency disorders or autoimmune disorders that require immunosuppressive therapy, and (k) free of solid organ transplantation or bone marrow transplantation.

In some embodiments, the human patient is monitored for at least 28 days for development of toxicity after each administration of the population of genetically engineered T cells. In some embodiments, the human patient is subject to toxicity management if development of toxicity is observed. In some embodiments, the human patient is an adult.

In some embodiments, the CAR that binds CD70 comprises an extracellular domain, a CD8 transmembrane domain, a 4-1BB co-stimulatory domain, and a CD3ζ cytoplasmic signaling domain, and wherein the extracellular domain is a single-chain antibody fragment (scFv) that binds CD70. In some embodiments, the scFv comprises a heavy chain variable domain (VH) comprising SEQ ID NO: 49, and a light chain variable domain (VL) comprising SEQ ID NO: 50. In some embodiments, the scFv comprises SEQ ID NO: 48. In some embodiments, the CAR comprises SEQ ID NO: 46.

In some embodiments, the disrupted TRAC gene is produced by a CRISPR/Cas9 gene editing system, which comprises a guide RNA comprising a spacer sequence of SEQ ID NO:

8 or 9. In some embodiments, the disrupted TRAC gene has a deletion of the region targeted by the spacer sequence of SEQ ID NO: 8, or a portion thereof.

In some embodiments, the disrupted β2M gene is produced by a CRISPR/Cas9 gene editing system, which comprises a guide RNA comprising a spacer sequence of SEQ ID NO: 12 or 13.

In some embodiments, the disrupted CD70 gene is produced by a CRISPR/Cas9 gene editing system, which comprises a guide RNA comprising a spacer sequence of SEQ ID NO:

4.

### BRIEF DESCRIPTION OF THE DRAWINGS

FIG. 1 includes graphs showing efficient multiple gene editing in TRAC⁻/β2M⁻/CD70⁻/anti-CD70 CAR⁺ (i.e., 3×KO, CD70 CAR⁺) T cells.

FIG. 2 includes a graph showing that normal proportions of CD4+ and CD8+ T cells are maintained among the TRAC⁻/β2M⁻/CD70⁻/anti-CD70 CAR⁺ T cell population.

FIG. 3 includes a graph showing robust cell expansion in TRAC⁻/β2M⁻/CD70⁻/anti-CD70 CAR⁺ T cells. The total number of viable cells was quantified in 3×KO (TRAC−/β2M−/CD70−) and 2×KO (TRAC−/β2M−) anti-CD70 CAR T cells. 3×KO cells were generated with either CD70 sgRNA T7 or T8.

FIG. 4 includes a graph showing robust cell killing of A498 cells by 3×KO (TRAC⁻/β2M⁻/CD70⁻) anti-CD70 CAR⁺ T cells compared to 2×KO (TRAC⁻/β2M⁻) anti-CD70 CAR⁺ T cells.

FIG. 5 includes a graph showing A498 cell killing by anti-CD70 CAR T cells after serial rechallenge. 3×KO (TRAC⁻/β2M⁻/CD70⁻) and the development lot of CTX130 cells (CTX130) anti-CD70 CAR+ T cells were utilized.

FIGS. 6A-6C include graphs showing results from testing of the development lot of CTX130 cells (lot 01) for cytokine secretion in the presence of CD70+ renal cell carcinoma cells. CTX130 cells were co-cultured with CD70+ (A498; FIG. 6A or ACHN; FIG. 6B) or CD70− (MCF7; FIG. 6C) target cells at the indicated ratios. Unedited T cells were used as control T cells. IFN-γ (left) and IL-2 (right) levels were determined. Mean of biological triplicates±the standard deviation are shown.

FIGS. 7A-7C include graphs showing results from testing of the development lot of CTX130 cells (lot 01) for cell killing activity against CD70 high (A498; FIG. 7A), CD70 low (ACHN; FIG. 7B), and CD70 negative (MCF7; FIG. 7C) cells lines at multiple T cell to target cell ratios. Each data point represents data from triplicates±the standard deviation. Negative values are shown as zero.

FIGS. 8A-8D includes graphs showing results from testing CTX130 cells in various subcutaneous renal cell carcinoma tumor xenograft models. FIG. 8A: a subcutaneous A498-NOG model. FIG. 8B: a subcutaneous 786-O-NSG model. FIG. 8C: a subcutaneous Caki-2-NSG model. FIG. 8D: a subcutaneous Caki-1-NSG model. Tumor volumes were measured twice weekly for the duration of the study. Each point represents the mean tumor volume±standard error.

FIG. 9 includes a graph showing results from testing the efficacy of CTX130 cells in a subcutaneous A498 xenograft model with tumor re-challenge. Tumors were allowed to grow to an average size of approximately 51 mm³ after which the tumor-bearing mice were randomized in two groups (N=5/group). Group 1 was left untreated while Group 2 received 7×10⁶ CAR+ CTX130 cells and Group 3 received 8×10⁶ CAR+ TRAC− B2M− Anti-CD70 CAR T cells. On Day 25, a tumor re-challenge was initiated whereby 5×10⁶ A498 cells were injected into the left flank of treated mice and into a new control group (Group 4). Tumor volume was measured twice weekly for the duration of the study. Each point represents the mean tumor volume±standard error.

FIG. 10 includes a graph showing results from testing the efficacy of CTX130 cells in a subcutaneous A498 xenograft model with redosing of CTX130 cells. When mean tumor size reached an average size of approximately 453 mm³, mice were either left untreated or injected intravenously (N=5) with 8.6×10⁶ CAR+ CTX130 cells per mouse. Group 2 mice were treated with a second and third dose of 8.6×10⁶ CAR+ CTX130 cells per mouse on day 17 and 36, respectively. Group 3 mice were treated with a second dose of 8.6×10⁶ CAR+ CTX130 cells per mouse on day 36. Tumor volumes were measured twice weekly for the duration of the study. Each point represents the mean tumor volume±standard error.

FIG. 11 is a schematic depicting the clinical study design to evaluate CTX130 cells administration to subjects with renal cell carcinoma (RCC). DLT assessment is part of Acute Toxicity Monitoring, but the DLT-assessment period is only 28 days. DLT: dose-limiting toxicity; M: month; max: maximum; min: minimum.

The details of one or more embodiments of the invention are set forth in the description below. Other features or advantages of the present invention will be apparent from the following drawings and detailed description of several embodiments, and also from the appended claims.

### DETAILED DESCRIPTION

Renal cancer accounts for approximately 2% to 3% of all cancer diagnoses and cancer deaths worldwide, with incidence rates generally higher in developed countries (Ferlay et al., Eur J Cancer, 49, 1374-403, 2013). Renal cancer is among the 10 most common cancers in both men and women. Worldwide, there are an estimated 209,000 newly diagnosed cases of RCC and 102,000 deaths from RCC per year (Rini et al., N Engl J Med, 380, 1116-1127, 2019).

Localized RCC can be treated with partial or radical nephrectomy, ablation, or under certain circumstances with active surveillance. Despite the curative intent of a nephrectomy, —30% of patients with localized ccRCC eventually develop metastases (Frank et al., J Urol, 168, 2395-400, 2002, and Patard et al., J Clin Oncol, 22, 3316-22, 2004) requiring systemic therapy, and most of these relapsed patients will ultimately face death from renal cancer.

Checkpoint inhibitors (CPIs) have recently been approved as first-line systemic therapy for patients with unresectable or metastatic RCC. Yet, patients who relapse after treatment with CPIs have no treatment options with established life-prolonging benefit, and thus are in need of new treatment alternatives.

Surprisingly, the anti-CD70 CAR+ T cells as disclosed herein successfully reduced tumor burden in various subcutaneous renal cell carcinoma (RCC) xenograft models and displayed long-term in vivo efficacy that prevented tumor growth after re-exposure to tumor cells. Significant reductions in tumor burden were also observed after redosing of anti-CD70 CAR T cells.

Accordingly, the present disclosure provides, in some aspects, therapeutic uses of anti-CD70 CAR+ T cells (for example, the CTX130 cells) for treating RCC. The anti-CD70 CART cells, methods of producing such (e.g., via the CRISPR approach), as well as components and processes (e.g., the CRISPR approach for gene editing and components used therein) for making the anti-CD70 CAR+ T cells disclosed herein are also within the scope of the present disclosure.

### I. Anti-CD70 Allogeneic CAR T Cells

Disclosed herein are anti-CD70 CAR T cells (e.g., CTX130 cells) for use in treating renal cell carcinoma (RCC). In some embodiments, the anti-CD70 CAR T cells are allogeneic T cells having a disrupted TRAC gene, a disrupted B2M gene, a disrupted CD70 gene, or a combination thereof. In specific examples, the anti-CD70 CAR T cells express an anti-CD70 CAR and have endogenous TRAC, B2M, and CD70 genes disrupted. Any suitable gene editing methods known in the art can be used for making the anti-CD70 CAR T cells disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9).

Exemplary genetic modifications of the anti-CD70 CAR T cells include targeted disruption of T cell receptor alpha constant (TRAC), β2M, CD70, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The disruption of CD70 results in loss of expression of CD70, which prevents possible cell-to-cell fratricide prior to insertion of the CD70 CAR. The addition of the anti-CD70 CAR directs the modified T cells towards CD70− expressing tumor cells.

The anti-CD70 CAR may comprise an anti-CD70 single-chain variable fragment (scFv) specific for CD70, followed by hinge domain and transmembrane domain (e.g., a CD8 hinge and transmembrane domain) that is fused to an intracellular co-signaling domain (e.g., a 4-1BB co-stimulatory domain) and a CD3ζ signaling domain.

### (i) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include MLLLVTSLLLCELPHPAFLLIP (SEQ ID NO: 52) and MALPVTALLLPLALLLHAARP (SEQ ID NO: 53). Other signal peptides may be used.
(a) Antigen Binding Extracellular Domain The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

In some examples, the CAR constructs disclosed herein comprise a scFv extracellular domain capable of binding to CD70. An example of an anti-CD70 CAR is provided in Examples below.

(b) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain Other transmembrane domains may be used as provided herein. In some embodiments, the transmembrane domain is a CD8a transmembrane domain containing the sequence of FVPVFLPAKPTTTPAPRPPTPAPTIASQPLSLRPEACRPAAGG AVHTRGLDFACDIYIWAPLAGTCGVLLLSLVITLYCNHRNR (SEQ ID NO: 54) or IYIWAPLAGTCGVLLLSLVITLY (SEQ ID NO: 55). Other transmembrane domains may be used.

(c) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(d) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

It should be understood that methods described herein encompasses more than one suitable CAR that can be used to produce genetically engineered T cells expressing the CAR, for example, those known in the art or disclosed herein. Examples can be found in, e.g., WO 2019/097305A2, and WO2019/215500, the relevant disclosures of each of the prior applications are incorporated by reference herein for the purpose and subject matter referenced herein.

For example, the CAR binds CD70 (also known as a “CD70 CAR” or an “anti-CD70 CAR”). The amino acid sequence of an exemplary CAR that binds CD70 is provided in SEQ ID NO: 46.

TABLE 1
Sequences of Exemplary Anti-CD70 CAR Construct Components.

| | | SEQ ID |
|---|---|---|
| Description | Sequence | NO: |
| CD70 | CCTGCAGGCAGCTGCGCGCTCGCTCGCTCACTGAGGCCGCCCGGGCGTCGG | 43 |
| rAAV | GCGACCTTTGGTCGCCCGGCCTCAGTGAGCGAGCGAGCGCGCAGAGAGGGA | |
| (CD70B scFv | GTGGCCAACTCCATCACTAGGGGTTCCTGCGGCCGCACGCGTGAGATGTAA | |
| with 41BB) | GGAGCTGCTGTGACTTGCTCAAGGCCTTATATCGAGTAAACGGTAGTGCTG | |
| | GGGCTTAGACGCAGGTGTTCTGATTTATAGTTCAAAACCTCTATCAATGAG | |
| | AGAGCAATCTCCTGGTAATGTGATAGATTTCCCAACTTAATGCCAACATAC | |
| | CATAAACCTCCCATTCTGCTAATGCCCAGCCTAAGTTGGGGAGACCACTCC | |
| | AGATTCCAAGATGTACAGTTTGCTTTGCTGGGCCTTTTTCCCATGCCTGCC | |
| | TTTACTCTGCCAGAGTTATATTGCTGGGGTTTTGAAGAAGATCCTATTAAA | |
| | TAAAAGAATAAGCAGTATTATTAAGTAGCCCTGCATTTCAGGTTTCCTTGA | |
| | GTGGCAGGCCAGGCCTGGCCGTGAACGTTCACTGAAATCATGGCCTCTTGG | |
| | CCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCC | |
| | AGCCCCACAGAGCCCCGCCCTTGTCCATCACTGGCATCTGGACTCCAGCCT | |
| | GGGTTGGGGCAAAGAGGGAAATGAGATCATGTCCTAACCCTGATCCTCTTG | |
| | TCCCACAGATATCCAGAACCCTGACCCTGCCGTGTACCAGCTGAGAGACTC | |
| | TAAATCCAGTGACAAGTCTGTCTGCCTATTCACCGATTTTGATTCTCAAAC | |
| | AAATGTGTCACAAAGTAAGGATTCTGATGTGTATATCACAGACAAAACTGT | |
| | GCTAGACATGAGGTCTATGGACTTCAGGCTCCGGTGCCCGTCAGTGGGCAG | |
| | AGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCG | |
| | TGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACAC | |
| | AGGTAAGTGCCGTGTGTGGTTCCCGCGGGCCTGGCCTCTTTACGGGTTATG | |
| | GCCCTTGCGTGCCTTGAATTACTTCCACTGGCTGCAGTACGTGATTCTTGA | |
| | TCCCGAGCTTCGGGTTGGAAGTGGGTGGGAGAGTTCGAGGCCTTGCGCTTA | |
| | AGGAGCCCCTTCGCCTCGTGCTTGAGTTGAGGCCTGGCCTGGGCGCTGGGG | |
| | CCGCCGCGTGCGAATCTGGTGGCACCTTCGCGCCTGTCTCGCTGCTTTCGA | |
| | TAAGTCTCTAGCCATTTAAAATTTTTGATGACCTGCTGCGACGCTTTTTTT | |
| | CTGGCAAGATAGTCTTGTAAATGCGGGCCAAGATCTGCACACTGGTATTTC | |
| | GGTTTTTGGGGCCGCGGGCGGCGACGGGGCCCGTGCGTCCCAGCGCACATG | |
| | TTCGGCGAGGCGGGGCCTGCGAGCGCGGCCACCGAGAATCGGACGGGGGTA | |
| | GTCTCAAGCTGGCCGGCCTGCTCTGGTGCCTGGCCTCGCGCCGCCGTGTAT | |
| | CGCCCCGCCCTGGGCGGCAAGGCTGGCCCGGTCGGCACCAGTTGCGTGAGC | |
| | GGAAAGATGGCCGCTTCCCGGCCCTGCTGCAGGGAGCTCAAAATGGAGGAC | |
| | GCGGCGCTCGGGAGAGCGGGCGGGTGAGTCACCCACACAAAGGAAAAGGGC | |
| | CTTTCCGTCCTCAGCCGTCGCTTCATGTGACTCCACGGAGTACCGGGCGCC | |
| | GTCCAGGCACCTCGATTAGTTCTCGAGCTTTTGGAGTACGTCGTCTTTAGG | |
| | TTGGGGGGAGGGGTTTTATGCGATGGAGTTTCCCCACACTGAGTGGGTGGA | |
| | GACTGAAGTTAGGCCAGCTTGGCACTTGATGTAATTCTCCTTGGAATTTGC | |
| | CCTTTTTGAGTTTGGATCTTGGTTCATTCTCAAGCCTCAGACAGTGGTTCA | |
| | AAGTTTTTTTCTTCCATTTCAGGTGTCGTGACCACCATGGCGCTTCCGGTG | |
| | ACAGCACTGCTCCTCCCCTTGGCGCTGTTGCTCCACGCAGCAAGGCCGCAG | |
| | GTCCAGTTGGTGCAAAGCGGGGCGGAGGTGAAAAAACCCGGCGCTTCCGTG | |
| | AAGGTGTCCTGTAAGGCGTCCGGTTATACGTTCACGAACTACGGGATGAAT | |
| | TGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACT | |
| | ATGACGCGCGATACCAGCATATCCACCGCATACATGGAGCTGTCCCGACTC | |
| | CGGTCAGACGACACGGCTGTCTACTATTGTGCTCGGGACTATGGCGATTAT | |
| | GGCATGGACTACTGGGGTCAGGGTACGACTGTAACAGTTAGTAGTGGTGGA | |
| | GGCGGCAGTGGCGGGGGGGGAAGCGGAGGAGGGGGTTCTGGTGACATAGTT | |
| | ATGACCCAATCCCCAGATAGTTTGGCGGTTTCTCTGGGCGAGAGGGCAACG | |
| | ATTAATTGTCGCGCATCAAAGAGCGTTTCAACGAGCGGATATTCTTTTATG | |
| | CATTGGTACCAGCAAAAACCCGGACAACCGCCGAAGCTGCTGATCTACTTG | |
| | GCTTCAAATCTTGAGTCTGGGGTGCCGGACCGATTTTCTGGTAGTGGAAGC | |
| | GGAACTGACTTTACGCTCACGATCAGTTCACTGCAGGCTGAGGATGTAGCG | |
| | GTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCA | |
| | GCCAAACCGACCACGACTCCCGCCCCGCGCCCTCCGACACCCGCTCCCACC | |
| | ATCGCCTCTCAACCTCTTAGTCTTCGCCCCGAGGCATGCCGACCCGCCGCC | |
| | GGGGGTGCTGTTCATACGAGGGGCTTGGACTTCGCTTGTGATATTTACATT | |
| | TGGGCTCCGTTGGCGGGTACGTGCGGCGTCCTTTTGTTGTCACTCGTTATT | |
| | ACTTTGTATTGTAATCACAGGAATCGCAAACGGGGCAGAAAGAAACTCCTG | |
| | TATATATTCAAACAACCATTTATGAGACCAGTACAAACTACTCAAGAGGAA | |
| | GATGGCTGTAGCTGCCGATTTCCAGAAGAAGAAGAAGGAGGATGTGAACTG | |
| | CGAGTGAAGTTTTCCCGAAGCGCAGACGCTCCGGCATATCAGCAAGGACAG | |
| | AATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAAGA | |
| | AAGAATCCCCAAGAAGGACTCTACAATGAACTCCAGAAGGATAAGATGGCG | |
| | GAGGCCTACTCAGAAATAGGTATGAAGGGCGAACGACGACGGGGAAAAGGT | |
| | CACGATGGCCTCTACCAAGGGTTGAGTACGGCAACCAAAGATACGTACGAT | |
| | GCACTGCATATGCAGGCCCTGCCTCCCAGATAATAATAAAATCGCTATCCA | |
| | TCGAAGATGGATGTGTGTTGGTTTTTTGTGTGTGGAGCAACAAATCTGACT | |
| | TTGCATGTGCAAACGCCTTCAACAACAGCATTATTCCAGAAGACACCTTCT | |
| | TCCCCAGCCCAGGTAAGGGCAGCTTTGGTGCCTTCGCAGGCTGTTTCCTTG | |
| | CTTCAGGAATGGCCAGGTTCTGCCCAGAGCTCTGGTCAATGATGTCTAAAA | |
| | CTCCTCTGATTGGTGGTCTCGGCCTTATCCATTGCCACCAAAACCCTCTTT | |
| | TTACTAAGAAACAGTGAGCCTTGTTCTGGCAGTCCAGAGAATGACACGGGA | |
| | AAAAAGCAGATGAAGAGAAGGTGGCAGGAGAGGGCACGTGGCCCAGCCTCA | |
| | GTCTCTCCAACTGAGTTCCTGCCTGCCTGCCTTTGCTCAGACTGTTTGCCC | |
| | CTTACTGCTCTTCTAGGCCTCATTCTAAGCCCCTTCTCCAAGTTGCCTCTC | |
| | CTTATTTCTCCCTGTCTGCCAAAAAATCTTTCCCAGCTCACTAAGTCAGTC | |
| | TCACGCAGTCACTCATTAACCCACCAATCACTGATTGTGCCGGCACATGAA | |
| | TGCACCAGGTGTTGAAGTGGAGGAATTAAAAAGTCAGATGAGGGGTGTGCC | |
| | CAGAGGAAGCACCATTCTAGTTGGGGGAGCCCATCTGTCAGCTGGGAAAAG | |
| | TCCAAATAACTTCAGATTGGAATGTGTTTTAACTCAGGGTTGAGAAAACAG | |
| | CTACCTTCAGGACAAAAGTCAGGGAAGGGCTCTCTGAAGAAATGCTACTTG | |
| | AAGATACCAGCCCTACCAAGGGCAGGGAGAGGACCCTATAGAGGCCTGGGA | |
| | CAGGAGCTCAATGAGAAAGGTAACCACGTGCGGACCGAGGCTGCAGCGTCG | |
| | TCCTCCCTAGGAACCCCTAGTGATGGAGTTGGCCACTCCCTCTCTGCGCGC | |
| | TCGCTCGCTCACTGAGGCCGGGCGACCAAAGGTCGCCCGACGCCCGGGCTT | |
| | TGCCCGGGCGGCCTCAGTGAGCGAGCGAGCGCGCAGCTGCCTGCAGG | |
| CD70 | GAGATGTAAGGAGCTGCTGTGACTTGCTCAAGGCCTTATATCGAGTAAACG | 44 |
| LHA to RHA | GTAGTGCTGGGGCTTAGACGCAGGTGTTCTGATTTATAGTTCAAAACCTCT | |
| (CD70B scFv | ATCAATGAGAGAGCAATCTCCTGGTAATGTGATAGATTTCCCAACTTAATG | |
| with 41BB) | CCAACATACCATAAACCTCCCATTCTGCTAATGCCCAGCCTAAGTTGGGGA | |
| | GACCACTCCAGATTCCAAGATGTACAGTTTGCTTTGCTGGGCCTTTTTCCC | |
| | ATGCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCAGG | |
| | TTTCCTTGAGTGGCAGGCCAGGCCTGGCCGTGAACGTTCACTGAAATCATG | |
| | GCCTCTTGGCCAAGATTGATAGCTTGTGCCTGTCCCTGAGTCCCAGTCCAT | |
| | CACGAGCAGCTGGTTTCTAAGATGCTATTTCCCGTATAAAGCATGAGACCG | |
| | TGACTTGCCAGCCCCACAGAGCCCCGCCCTTGTCCATCACTGGCATCTGGA | |
| | CTCCAGCCTGGGTTGGGGCAAAGAGGGAAATGAGATCATGTCCTAACCCTG | |
| | ATCCTCTTGTCCCACAGATATCCAGAACCCTGACCCTGCCGTGTACCAGCT | |
| | GAGAGACTCTAAATCCAGTGACAAGTCTGTCTGCCTATTCACCGATTTTGA | |
| | TTCTCAAACAAATGTGTCACAAAGTAAGGATTCTGATGTGTATATCACAGA | |
| | CAAAACTGTGCTAGACATGAGGTCTATGGACTTCAGGCTCCGGTGCCCGTC | |
| | AGTGGGCAGAGCGCACATCGCCCACAGTCCCCGAGAAGTTGGGGGGAGGGG | |
| | TCGGCAATTGAACCGGTGCCTAGAGAAGGTGGCGCGGGGTAAACTGGGAAA | |
| | GTGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCCG | |
| | CCAGAACACAGGTAAGTGCCGTGTGTGGTTCCCGCGGGCCTGGCCTCTTTA | |
| | CGGGTTATGGCCCTTGCGTGCCTTGAATTACTTCCACTGGCTGCAGTACGT | |
| | GATTCTTGATCCCGAGCTTCGGGTTGGAAGTGGGTGGGAGAGTTCGAGGCC | |
| | TTGCGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTCTCGC | |
| | TGCTTTCGATAAGTCTCTAGCCATTTAAAATTTTTGATGACCTGCTGCGAC | |
| | GCTTTTTTTCTGGCAAGATAGTCTTGTAAATGCGGGCCAAGATCTGCACAC | |
| | TGGTATTTCGGTTTTTGGGGCCGCGGGCGGCGACGGGGCCCGTGCGTCCCA | |
| | GCGCACATGTTCGGCGAGGCGGGGCCTGCGAGCGCGGCCACCGAGAATCGG | |
| | ACGGGGGTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGCACCAGT | |
| | TGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAG | |
| | GAAAAGGGCCTTTCCGTCCTCAGCCGTCGCTTCATGTGACTCCACGGAGTA | |
| | CCGGGCGCCGTCCAGGCACCTCGATTAGTTCTCGAGCTTTTGGAGTACGTC | |
| | GTCTTTAGGTTGGGGGGAGGGGTTTTATGCGATGGAGTTTCCCCACACTGA | |
| | GTGGGTGGAGACTGAAGTTAGGCCAGCTTGGCACTTGATGTAATTCTCCTT | |
| | GGAATTTGCCCTTTTTGAGTTTGGATCTTGGTTCATTCTCAAGCCTCAGAC | |
| | AGTGGTTCAAAGTTTTTTTCTTCCATTTCAGGTGTCGTGACCACCATGGCG | |
| | CTTCCGGTGACAGCACTGCTCCTCCCCTTGGCGCTGTTGCTCCACGCAGCA | |
| | AGGCCGCAGGTCCAGTTGGTGCAAAGCGGGGCGGAGGTGAAAAAACCCGGC | |
| | GCTTCCGTGAAGGTGTCCTGTAAGGCGTCCGGTTATACGTTCACGAACTAC | |
| | GGGATGAATTGGGTTCGCCAAGCGCCGGGGCAGGGACTGAAATGGATGGGG | |
| | TGGATAAATACCTACACCGGCGAACCTACATACGCCGACGCTTTTAAAGGG | |
| | CGAGTCACTATGACGCGCGATACCAGCATATCCACCGCATACATGGAGCTG | |
| | TCCCGACTCCGGTCAGACGACACGGCTGTCTACTATTGTGCTCGGGACTAT | |
| | GGCGATTATGGCATGGACTACTGGGGTCAGGGTACGACTGTAACAGTTAGT | |
| | AGTGGTGGAGGCGGCAGTGGCGGGGGGGGAAGCGGAGGAGGGGGTTCTGGT | |
| | GACATAGTTATGACCCAATCCCCAGATAGTTTGGCGGTTTCTCTGGGCGAG | |
| | AGGGCAACGATTAATTGTCGCGCATCAAAGAGCGTTTCAACGAGCGGATAT | |
| | TCTTTTATGCATTGGTACCAGCAAAAACCCGGACAACCGCCGAAGCTGCTG | |
| | ATCTACTTGGCTTCAAATCTTGAGTCTGGGGTGCCGGACCGATTTTCTGGT | |
| | AGTGGAAGCGGAACTGACTTTACGCTCACGATCAGTTCACTGCAGGCTGAG | |
| | GATGTAGCGGTCTATTATTGCCAGCACAGTAGAGAAGTCCCCTGGACCTTC | |
| | GGTCAAGGCACGAAAGTAGAAATTAAAAGTGCTGCTGCCTTTGTCCCGGTA | |
| | TTTCTCCCAGCCAAACCGACCACGACTCCCGCCCCGCGCCCTCCGACACCC | |
| | GCTCCCACCATCGCCTCTCAACCTCTTAGTCTTCGCCCCGAGGCATGCCGA | |
| | CCCGCCGCCGGGGGTGCTGTTCATACGAGGGGCTTGGACTTCGCTTGTGAT | |
| | ATTTACATTTGGGCTCCGTTGGCGGGTACGTGCGGCGTCCTTTTGTTGTCA | |
| | CTCGTTATTACTTTGTATTGTAATCACAGGAATCGCAAACGGGGCAGAAAG | |
| | AAACTCCTGTATATATTCAAACAACCATTTATGAGACCAGTACAAACTACT | |
| | CAAGAGGAAGATGGCTGTAGCTGCCGATTTCCAGAAGAAGAAGAAGGAGGA | |
| | TGTGAACTGCGAGTGAAGTTTTCCCGAAGCGCAGACGCTCCGGCATATCAG | |
| | CAAGGACAGAATCAGCTGTATAACGAACTGAATTTGGGACGCCGCGAGGAG | |
| | TATGACGTGCTTGATAAACGCCGGGGGAGAGACCCGGAAATGGGGGGTAAA | |
| | CCCCGAAGAAAGAATCCCCAAGAAGGACTCTACAATGAACTCCAGAAGGAT | |
| | AAGATGGCGGAGGCCTACTCAGAAATAGGTATGAAGGGCGAACGACGACGG | |
| | GGAAAAGGTCACGATGGCCTCTACCAAGGGTTGAGTACGGCAACCAAAGAT | |
| | ACGTACGATGCACTGCATATGCAGGCCCTGCCTCCCAGATAATAATAAAAT | |
| | CGCTATCCATCGAAGATGGATGTGTGTTGGTTTTTTGTGTGTGGAGCAACA | |
| | AATCTGACTTTGCATGTGCAAACGCCTTCAACAACAGCATTATTCCAGAAG | |
| | ACACCTTCTTCCCCAGCCCAGGTAAGGGCAGCTTTGGTGCCTTCGCAGGCT | |
| | GTTTCCTTGCTTCAGGAATGGCCAGGTTCTGCCCAGAGCTCTGGTCAATGA | |
| | TGTCTAAAACTCCTCTGATTGGTGGTCTCGGCCTTATCCATTGCCACCAAA | |
| | ACCCTCTTTTTACTAAGAAACAGTGAGCCTTGTTCTGGCAGTCCAGAGAAT | |
| | GACACGGGAAAAAAGCAGATGAAGAGAAGGTGGCAGGAGAGGGCACGTGGC | |
| | CCAGCCTCAGTCTCTCCAACTGAGTTCCTGCCTGCCTGCCTTTGCTCAGAC | |
| | TGTTTGCCCCTTACTGCTCTTCTAGGCCTCATTCTAAGCCCCTTCTCCAAG | |
| | TTGCCTCTCCTTATTTCTCCCTGTCTGCCAAAAAATCTTTCCCAGCTCACT | |
| | AAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAG | |
| | GGGTGTGCCCAGAGGAAGCACCATTCTAGTTGGGGGAGCCCATCTGTCAGC | |
| | TGGGAAAAGTCCAAATAACTTCAGATTGGAATGTGTTTTAACTCAGGGTTG | |
| | AGAAAACAGCTACCTTCAGGACAAAAGTCAGGGAAGGGCTCTCTGAAGAAA | |
| | TGCTACTTGAAGATACCAGCCCTACCAAGGGCAGGGAGAGGACCCTATAGA | |
| | GGCCTGGGACAGGAGCTCAATGAGAAAGG | |
| CD70 CAR | ATGGCGCTTCCGGTGACAGCACTGCTCCTCCCCTTGGCGCTGTTGCTCCACG | 45 |
| nucleotide | CAGCAAGGCCGCAGGTCCAGTTGGTGCAAAGCGGGGCGGAGGTGAAAAAACC | |
| sequence | CGGCGCTTCCGTGAAGGTGTCCTGTAAGGCGTCCGGTTATACGTTCACGAAC | |
| (CD70B scFv with | TACGGGATGAATTGGGTTCGCCAAGCGCCGGGGCAGGGACTGAAATGGATGG | |
| 41BB) | GGTGGATAAATACCTACACCGGCGAACCTACATACGCCGACGCTTTTAAAGG | |
| | GCGAGTCACTATGACGCGCGATACCAGCATATCCACCGCATACATGGAGCTG | |
| | TCCCGACTCCGGTCAGACGACACGGCTGTCTACTATTGTGCTCGGGACTATG | |
| | GCGATTATGGCATGGACTACTGGGGTCAGGGTACGACTGTAACAGTTAGTAG | |
| | TGGTGGAGGCGGCAGTGGCGGGGGGGGAAGCGGAGGAGGGGGTTCTGGTGAC | |
| | ATAGTTATGACCCAATCCCCAGATAGTTTGGCGGTTTCTCTGGGCGAGAGGG | |
| | CAACGATTAATTGTCGCGCATCAAAGAGCGTTTCAACGAGCGGATATTCTTT | |
| | TATGCATTGGTACCAGCAAAAACCCGGACAACCGCCGAAGCTGCTGATCTAC | |
| | TTGGCTTCAAATCTTGAGTCTGGGGTGCCGGACCGATTTTCTGGTAGTGGAA | |
| | GCGGAACTGACTTTACGCTCACGATCAGTTCACTGCAGGCTGAGGATGTAGC | |
| | GGTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCAG | |
| | CCAAACCGACCACGACTCCCGCCCCGCGCCCTCCGACACCCGCTCCCACCAT | |
| | CGCCTCTCAACCTCTTAGTCTTCGCCCCGAGGCATGCCGACCCGCCGCCGGG | |
| | GGTGCTGTTCATACGAGGGGCTTGGACTTCGCTTGTGATATTTACATTTGGG | |
| | CTCCGTTGGCGGGTACGTGCGGCGTCCTTTTGTTGTCACTCGTTATTACTTT | |
| | GTATTGTAATCACAGGAATCGCAAACGGGGCAGAAAGAAACTCCTGTATATA | |
| | TTCAAACAACCATTTATGAGACCAGTACAAACTACTCAAGAGGAAGATGGCT | |
| | GTAGCTGCCGATTTCCAGAAGAAGAAGAAGGAGGATGTGAACTGCGAGTGAA | |
| | GTTTTCCCGAAGCGCAGACGCTCCGGCATATCAGCAAGGACAGAATCAGCTG | |
| | TATAACGAACTGAATTTGGGACGCCGCGAGGAGTATGACGTGCTTGATAAAC | |
| | GCCGGGGGAGAGACCCGGAAATGGGGGGTAAACCCCGAAGAAAGAATCCCCA | |
| | AGAAGGACTCTACAATGAACTCCAGAAGGATAAGATGGCGGAGGCCTACTCA | |
| | GAAATAGGTATGAAGGGCGAACGACGACGGGGAAAAGGTCACGATGGCCTCT | |
| | ACCAAGGGTTGAGTACGGCAACCAAAGATACGTACGATGCACTGCATATGCA | |
| | GGCCCTGCCTCCCAGATAA | |
| CD70 CAR amino | MALPVTALLLPLALLLHAARPQVQLVQSGAEVKKPGASVKVSCKASGYTFTN | 46 |
| acid sequence | YGMNWVRQAPGQGLKWMGWINTYTGEPTYADAFKGRVTMTRDTSISTAYMEL | |
| (CD70B scFv with | SRLRSDDTAVYYCARDYGDYGMDYWGQGTTVTVSSGGGGSGGGGSGGGGSGD | |
| 41BB) | IVMTQSPDSLAVSLGERATINCRASKSVSTSGYSFMHWYQQKPGQPPKLLIY | |
| | LASNLESGVPDRFSGSGSGTDFTLTISSLQAEDVAVYYCQHSREVPWTFGQG | |
| | TKVEIKSAAAFVPVFLPAKPTTTPAPRPPTPAPTIASQPLSLRPEACRPAAG | |
| | GAVHTRGLDFACDIYIWAPLAGTCGVLLLSLVITLYCNHRNRKRGRKKLLYI | |
| | FKQPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| CD70B | CAGGTCCAGTTGGTGCAAAGCGGGGCGGAGGTGAAAAAACCCGGCGCTTCCG | 47 |
| scFv nucleotide | TGAAGGTGTCCTGTAAGGCGTCCGGTTATACGTTCACGAACTACGGGATGAA | |
| sequence | TTGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACTA | |
| | TGACGCGCGATACCAGCATATCCACCGCATACATGGAGCTGTCCCGACTCCG | |
| | GTCAGACGACACGGCTGTCTACTATTGTGCTCGGGACTATGGCGATTATGGC | |
| | ATGGACTACTGGGGTCAGGGTACGACTGTAACAGTTAGTAGTGGTGGAGGCG | |
| | GCAGTGGCGGGGGGGGAAGCGGAGGAGGGGGTTCTGGTGACATAGTTATGAC | |
| | CCAATCCCCAGATAGTTTGGCGGTTTCTCTGGGCGAGAGGGCAACGATTAAT | |
| | TGTCGCGCATCAAAGAGCGTTTCAACGAGCGGATATTCTTTTATGCATTGGT | |
| | ACCAGCAAAAACCCGGACAACCGCCGAAGCTGCTGATCTACTTGGCTTCAAA | |
| | TCTTGAGTCTGGGGTGCCGGACCGATTTTCTGGTAGTGGAAGCGGAACTGAC | |
| | TTTACGCTCACGATCAGTTCACTGCAGGCTGAGGATGTAGCGGTCTATTATT | |
| | GCCAGCACAGTAGAGAAGTCCCCTGGACCTTCGGTCAAGGCACGAAAGTAGA | |
| | AATTAAA | |
| CD70B | QVQLVQSGAEVKKPGASVKVSCKASGYTFTNYGMNWVRQAPGQGLKWMGWIN | 48 |
| scFv amino acid | TYTGEPTYADAFKGRVTMTRDTSISTAYMELSRLRSDDTAVYYCARDYGDYG | |
| sequence | MDYWGQGTTVTVSSGGGGSGGGGSGGGGSGDIVMTQSPDSLAVSLGERATIN | |
| (linker | CRASKSVSTSGYSFMHWYQQKPGQPPKLLIYLASNLESGVPDRFSGSGSGTD | |
| underlined) | FTLTISSLQAEDVAVYYCQHSREVPWTFGQGTKVEIK | |
| CD70 VH | QVQLVQSGAEVKKPGASVKVSCKASGYTFTNYGMNWVRQAPGQGLKWMGWIN | 49 |
| | TYTGEPTYADAFKGRVTMTRDTSISTAYMELSRLRSDDTAVYYCARDYGDYG | |
| | MDYWGQGTTVTVSS | |
| CD70 VL | DIVMTQSPDSLAVSLGERATINCRASKSVSTSGYSFMHWYQQKPGQPPKLLI | 50 |
| | YLASNLESGVPDRFSGSGSGTDFTLTISSLQAEDVAVYYCQHSREVPWTFGQ | |
| | GTKVEIK | |
| Linker | GGGGSGGGGSGGGGSG | 51 |
| signal peptide | MLLLVTSLLLCELPHPAFLLIP | 52 |
| signal peptide | MALPVTALLLPLALLLHAARP | 53 |
| CD8a | FVPVFLPAKPTTTPAPRPPTPAPTIASQPLSLRPEACRPAAGGAVHTRGLDF | 54 |
| transmembrane | ACDIYIWAPLAGTCGVLLLSLVITLYCNHRNR | |
| domain | | |
| CD8a | IYIWAPLAGTCGVLLLSLVITLY | 55 |
| transmembrane | | |
| 4-1BB nucleotide | AAACGGGGCAGAAAGAAACTCCTGTATATATTCAAACAACCATTTATGAGAC | 56 |
| sequence | CAGTACAAACTACTCAAGAGGAAGATGGCTGTAGCTGCCGATTTCCAGAAGA | |
| | AGAAGAAGGAGGATGTGAACTG | |
| 4-1BB amino acid | KRGRKKLLYIFKQPFMRPVQTTQEEDGCSCRFPEEEEGGCEL | 57 |
| sequence | | |
| CD28 nucleotide | TCAAAGCGGAGTAGGTTGTTGCATTCCGATTACATGAATATGACTCCTCGCC | 58 |
| sequence | GGCCTGGGCCGACAAGAAAACATTACCAACCCTATGCCCCCCCACGAGACTT | |
| | CGCTGCGTACAGGTCC | |
| CD28 amino acid | SKRSRLLHSDYMNMTPRRPGPTRKHYQPYAPPRDFAAYRS | 59 |
| sequence | | |
| CD3ζ nucleotide | CGAGTGAAGTTTTCCCGAAGCGCAGACGCTCCGGCATATCAGCAAGGACAGA | 60 |
| sequence | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGA | |
| CD3ζ amino acid | RVKFSRSADAPAYQQGQNQLYNELNLGRREEYDVLDKRRGRDPEMGGKPRRK | 61 |
| sequence | NPQEGLYNELQKDKMAEAYSEIGMKGERRRGKGHDGLYQGLSTATKDTYDAL | |
| | HMQALPPR | |
| TRAC-LHA | GAGATGTAAGGAGCTGCTGTGACTTGCTCAAGGCCTTATATCGAGTAAACGG | 62 |
| | TAGTGCTGGGGCTTAGACGCAGGTGTTCTGATTTATAGTTCAAAACCTCTAT | |
| | CAATGAGAGAGCAATCTCCTGGTAATGTGATAGATTTCCCAACTTAATGCCA | |
| | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCA | |
| EF1α promoter | GGCTCCGGTGCCCGTCAGTGGGCAGAGCGCACATCGCCCACAGTCCCCGAGA | 63 |
| | AGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCGCA | |
| | ACGGGTTTGCCGCCAGAACACAGGTAAGTGCCGTGTGTGGTTCCCGCGGGCC | |
| | TGGCCTCTTTACGGGTTATGGCCCTTGCGTGCCTTGAATTACTTCCACTGGC | |
| | TGCAGTACGTGATTCTTGATCCCGAGCTTCGGGTTGGAAGTGGGTGGGAGAG | |
| | TTCGAGGCCTTGCGCTTAAGGAGCCCCTTCGCCTCGTGCTTGAGTTGAGGCC | |
| | TGGCCTGGGCGCTGGGGCCGCCGCGTGCGAATCTGGTGGCACCTTCGCGCCT | |
| | GTCTCGCTGCTTTCGATAAGTCTCTAGCCATTTAAAATTTTTGATGACCTGC | |
| | TGCGACGCTTTTTTTCTGGCAAGATAGTCTTGTAAATGCGGGCCAAGATCTG | |
| | CACACTGGTATTTCGGTTTTTGGGGCCGCGGGCGGCGACGGGGCCCGTGCGT | |
| | CCCAGCGCACATGTTCGGCGAGGCGGGGCCTGCGAGCGCGGCCACCGAGAAT | |
| | CGGACGGGGGTAGTCTCAAGCTGGCCGGCCTGCTCTGGTGCCTGGCCTCGCG | |
| | CCGCCGTGTATCGCCCCGCCCTGGGCGGCAAGGCTGGCCCGGTCGGCACCAG | |
| | TTGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAGG | |
| | AAAAGGGCCTTTCCGTCCTCAGCCGTCGCTTCATGTGACTCCACGGAGTACC | |
| | GGGCGCCGTCCAGGCACCTCGATTAGTTCTCGAGCTTTTGGAGTACGTCGTC | |
| | TTTAGGTTGGGGGGAGGGGTTTTATGCGATGGAGTTTCCCCACACTGAGTGG | |
| | GTGGAGACTGAAGTTAGGCCAGCTTGGCACTTGATGTAATTCTCCTTGGAAT | |
| | TTGCCCTTTTTGAGTTTGGATCTTGGTTCATTCTCAAGCCTCAGACAGTGGT | |
| | TCAAAGTTTTTTTCTTCCATTTCAGGTGTCGTGA | |
| Synthetic poly(A) | AATAAAATCGCTATCCATCGAAGATGGATGTGTGTTGGTTTTTTGTGTG | 64 |
| signal | | |
| TRAC-RHA | TGGAGCAACAAATCTGACTTTGCATGTGCAAACGCCTTCAACAACAGCATTA | 65 |
| | TTCCAGAAGACACCTTCTTCCCCAGCCCAGGTAAGGGCAGCTTTGGTGCCTT | |
| | CGCAGGCTGTTTCCTTGCTTCAGGAATGGCCAGGTTCTGCCCAGAGCTCTGG | |
| | TCAATGATGTCTAAAACTCCTCTGATTGGTGGTCTCGGCCTTATCCATTGCC | |
| | ACCAAAACCCTCTTTTTACTAAGAAACAGTGAGCCTTGTTCTGGCAGTCCAG | |
| | AGAATGACACGGGAAAAAAGCAGATGAAGAGAAGGTGGCAGGAGAGGGCACG | |
| | TGGCCCAGCCTCAGTCTCTCCAACTGAGTTCCTGCCTGCCTGCCTTTGCTCA | |
| | GACTGTTTGCCCCTTACTGCTCTTCTAGGCCTCATTCTAAGCCCCTTCTCCA | |
| | AGTTGCCTCTCCTTATTTCTCCCTGTCTGCCAAAAAATCTTTCCCAGCTCAC | |
| | TAAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAGG | |
| | GGTGTGCCCAGAGGAAGCACCATTCTAGTTGGGGGAGCCCATCTGTCAGCTG | |
| | GGAAAAGTCCAAATAACTTCAGATTGGAATGTGTTTTAACTCAGGGTTGAGA | |
| | AAACAGCTACCTTCAGGACAAAAGTCAGGGAAGGGCTCTCTGAAGAAATGCT | |
| | ACTTGAAGATACCAGCCCTACCAAGGGCAGGGAGAGGACCCTATAGAGGCCT | |
| | GGGACAGGAGCTCAATGAGAAAGG | |

(ii) Knock-Out of TRAC, B2M, and/or CD70 Genes

The anti-CD70 CAR-T cells disclosed herein may further have a disrupted TRAC gene, a disrupted B2M gene, a disrupted CD70 gene, or a combination thereof. The disruption of the TRAC locus results in loss of expression of the T cell receptor (TCR) and is intended to reduce the probability of Graft versus Host Disease (GvHD), while the disruption of the β2 M locus results in lack of expression of the major histocompatibility complex type I (MHC I) proteins and is intended to improve persistence by reducing the probability of host rejection. The disruption of the CD70 gene would minimize the fratricide effect in producing the anti-CD70 CAR-T cells. Further, disruption of the CD70 gene unexpectedly increased health and activity of the resultant engineered T cells. The addition of the anti-CD70 CAR directs the modified T cells towards CD70− expressing tumor cells.

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

In some instances, the disrupted TRAC gene in the anti-CD70 CAR-T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 17, which is the target site of TRAC guide RNA TA-1. See sequence tables below. In some examples, the fragment of SEQ ID NO: 17 may be replaced by a nucleic acid encoding the anti-CD70 CAR. Such a disrupted TRAC gene may comprise the nucleotide sequence of SEQ ID NO: 44.

The disrupted B2M gene in the anti-CD70 CAR-T cells disclosed herein may be generated using the CRISPR/Cas technology. In some examples, a B2M gRNA provided in the sequence table below can be used. The disrupted B2M gene may comprise a nucleotide sequence of any one of SEQ ID NOs: 31-36. See Table 4 below.

Alternatively or in addition, the disrupted CD70 gene in the anti-CD70 CAR-T cells disclosed herein may be generated using the CRISPR/Cas technology. In some examples, a CD70 gRNA provided in the sequence table below can be used. The disrupted CD70 gene may comprise a nucleotide sequence of any one of SEQ ID NOs:37-42. See Table 5 below.

(iii) Exemplary Anti-CD70 CAR T Cells

In some examples, the anti-CD70 CAR T cells are CTX130 cells, which are CD70− directed T cells having disrupted TRAC gene, B2M gene, and CD70 gene. CTX130 cells can be produced via ex vivo genetic modification using CRISPR/Cas9 (Clustered Regularly Interspaced Short Palindromic Repeats/CRISPR associated protein 9) gene editing components (sgRNA and Cas9 nuclease).

Also within the scope of the present disclosure are populations of anti-CD70 CAR T cells (e.g., a population of CTX130 cells), which comprises genetically engineered cells (e.g., CRISPR-Cas9-mediated gene edited) expressing the anti-CD70 CAR disclosed herein and disrupted TRAC, B2M, and CD70 genes; and the nucleotide sequence encoding the anti-CD70 CAR is inserted into the TRAC locus.

It should be understood that gene disruption encompasses gene modification through gene editing (e.g., using CRISPR/Cas gene editing to insert or delete one or more nucleotides). As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

The examples provided herein describe generating edited T cells, and engineering the edit T cells to express a chimeric antigen receptor (CAR) that binds CD70, thereby producing anti-CD70 CAR T cells express an anti-CD70 CAR and have endogenous TRAC, β2M, and CD70 genes disrupted.

In specific instances, the anti-CD70 CAR+ T cells are CTX130 cells, which are produced using CRISPR technology to disrupt targeted genes, and adeno-associated virus (AAV) transduction to deliver the CAR construct. CRISPR-Cas9-mediated gene editing involves three guide RNAs (sgRNAs): CD70− 7 sgRNA (SEQ ID NO: 2) which targets the CD70 locus, TA-1 sgRNA (SEQ ID NO: 6) which targets the TRAC locus, and B2M-1 sgRNA (SEQ ID NO: 10) which targets the β2M locus. The anti-CD70 CAR of CTX130 cells is composed of an anti-CD70 single-chain antibody fragment (scFv) specific for CD70, followed by a CD8 hinge and transmembrane domain that is fused to an intracellular co-signaling domain of 4-1BB and a CD3ζ signaling domain. As such, CTX130 is a CD70− directed T cell immunotherapy comprised of allogeneic T cells that are genetically modified ex vivo using CRISPR/Cas9 gene editing components (sgRNA and Cas9 nuclease).

In some embodiments, at least 50% of a population of CTX130 cells may not express a detectable level of β2M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of CTX130 cells may not express a detectable level of TRAC surface protein. For example, at least 55%, at least 60%, at least 70%, at least 75%, at least 80%, at least 85%, at least 90%, or at least 95% of the engineered T cells of a population may not express a detectable level of TRAC surface protein. In some embodiments, 50%400%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein.

In some embodiments, at least 50% of a population of CTX130 cells may not express a detectable level of CD70 surface protein. For example, at least 55%, at least 60%, at least 70%, at least 75%, at least 80%, at least 85%, at least 90%, at least 95%, or at least 98% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, or 95%-100% of the engineered T cells of a population does not express a detectable level of CD70 surface protein.

In some embodiments, a substantial percentage of the population of CTX130 cells may comprise more than one gene edit, which results in a certain percentage of cells not expressing more than one gene and/or protein.

For example, at least 50% of a population of CTX130 cells may not express a detectable level of two surface proteins, e.g., does not express a detectable level of β2M and TRAC proteins, β2M and CD70 proteins, or TRAC and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of two surface proteins. In another example, at least 50% of a population of the CTX130 cells may not express a detectable level of all of the three target surface proteins β2M, TRAC, and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M, TRAC, and CD70 surface proteins.

In some embodiments, the population of CTX130 cells may comprise more than one gene edit (e.g., in more than one gene), which may be an edit described herein. For example, the population of CTX130 cells may comprise a disrupted TRAC gene via the CRISPR/Cas technology using guide RNA TA-1 (see also Table 2, SEQ ID NOS: 6-7). Alternatively or in addition, the population of CTX130 cells may comprise a disrupted β2M gene via CRISPR/Cas9 technology using the guide RNA of B2M-1 (see also Table 2, SEQ ID NOS: 10-11). Such CTX130 cells may comprise Indels in the β2M gene, which comprise one or more of the nucleotide sequences listed in Table 4. For example, the population of CTX130 cells may comprise a disrupted CD70 gene via the CRISPR/Cas technology using guide RNA CD70− 7 (see also Table 2, SEQ ID NOS: 2-3). Further, the population of the CTX130 cells may comprise Indels in the CD70 gene, which may comprise one or more nucleotide sequences listed in Table 5.

In some embodiments, the CTX130 cells may comprise a deletion in the TRAC gene relative to unmodified T cells. For example, the CTX130 cells may comprise a deletion of the fragment AGAGCAACAGTGCTGTGGCC (SEQ ID NO: 17) in the TRAC gene, or a portion of thereof, e.g., a fragment of SEQ ID NO: 17 comprising 1, 2, 3, 4, 5, 6, 7, 8, 9, 10, 11, 12, 13, 14, 15, 16, 17, 18, or 19 consecutive base pairs. In some embodiments, the CTX130 cells include a deletion comprising the fragment of SEQ ID NO: 17 in the TRAC gene. In some embodiments, an engineered T cell comprises a deletion of SEQ ID NO: 17 in the TRAC gene relative to unmodified T cells. In some embodiments, an engineered T cell comprises a deletion comprising SEQ ID NO: 17 in the TRAC gene relative to unmodified T cells.

Further, the population of CTX130 cells may comprise cells expressing an anti-CD70 CAR such as those disclosed herein (e.g., SEQ ID NO: 46). The coding sequence of the anti-CD70 CAR may be inserted into the TRAC locus, e.g., at the region targeted by guide RNA TA-1 (see also Table 2, SEQ ID NOS: 6-7). In such instances, the amino acid sequence of the exemplary anti-CD70 CAR comprises the amino acid sequence of SEQ ID NO: 46.

In some embodiments, at least 30% at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of the CTX130 cells are CAR+ cells, which express the anti-CD70 CAR. See also WO 2019/097305A2, and WO2019/215500, the relevant disclosures of each of which are incorporated by reference for the subject matter and purpose referenced herein.

In specific examples, the anti-CD70 CAR-T cells disclosed herein (e.g., CTX130 cells) is a population of T cells having ≥30% CAR+ T cells, ≤0.4% TCR+ T cells, ≤30% B2M+ T cells, and ≤2% CD70+ T cells.

### (v) Pharmaceutical Compositions

In some aspects, the present disclosure provides pharmaceutical compositions comprising any of the populations of genetically engineered anti-CD70 CAR T cells as disclosed herein, for example, CTX130 cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered anti-CD70 CAR-T cells (e.g., CTX130 cells) suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxyethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered anti-CD70 CAR-T cells such as the CTX130 cells suspended in a cryopreservation solution (e.g., substantially free of serum) may be placed in storage vials.

Any of the pharmaceutical compositions disclosed herein, comprising a population of genetically engineered anti-CD70 CAR T cells as also disclosed herein (e.g., CTX130 cells), which optionally may be suspended in a cryopreservation solution as disclosed herein may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135° C. No significant changes were observed with respect to appearance, cell count, viability, % CAR⁺ T cells, % TCR⁺ T cells, % B2M⁺ T cells, and % CD70⁺ T cells after the cells have been stored under such conditions for a period of time.

In some embodiments, the pharmaceutical composition disclosed herein can be a suspension for infusion, comprising the anti-CD70 CAR T cells disclosed herein such as the CTX130 cells. In some examples, the suspension may comprise abut 25-85×10⁶ cells/ml (e.g., 50×10⁶ cells/ml) with ≥30% CAR+ T cells, ≤0.4% TCR+ T cells, ≤30% B2M+ T cells, and ≤2% CD70+ T cells. In some examples, the suspension may comprise about 25×10⁶ CAR+ cells/ml. In specific examples, the pharmaceutical composition may be placed in a vial, each comprising about 1.5×10⁸ CAR+ T cells such as CTX130 cells (e.g., viable cells). In other examples, the pharmaceutical composition may be placed in a vial, each comprising about 3×10⁸ CAR+ T cells such as CTX130 cells (e.g., viable cells).

### II. Preparation of Anti-CD70 CAR T Cells

Any suitable gene editing methods known in the art can be used for making the genetically engineered immune cells (e.g., T cells such as CTX130 cells) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as CTX130 cells are produced by the CRISPR technology in combination with homologous recombination using an adeno-associated viral vector (AAV) as a donor template.

### (i) CRISPR-Cas9-Mediated Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 1.

Amino acid sequence of Cas9 nuclease (SEQ ID NO:
1):
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a CD70 gene or a TRAC gene or a β2 M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a CD70 gene is provided in SEQ ID NO: 2. See also WO2019/215500, the relevant disclosures of which are incorporated by reference herein for the subject matter and purpose referenced herein. Other gRNA sequences may be designed using the CD70 gene sequence located on chromosome 19 (GRCh38: chromosome 19: 6,583,183-6,604,103; Ensembl; ENSG00000125726). In some embodiments, gRNAs targeting the CD70 genomic region and Cas9 create breaks in the CD70 genomic region resulting Indels in the CD70 gene disrupting expression of the mRNA or protein.

An exemplary gRNA targeting a TRAC gene is provided in SEQ ID NO: 6. See also WO 2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

An exemplary gRNA targeting a β2M gene is provided in SEQ ID NO: 10. See also WO2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

TABLE 2
sgRNA Sequences and Target Gene Sequences.

SEQ ID
NO:

sgRNA Sequences

| | | | |
|---|---|---|---|
| CD70 | Modified | G*C*U*UUGGUCCCAUUGGUCGCguuuuagagcuagaaauagcaa | 2 |
| sgRNA | | guuaaaauaaggcuaguccguuaucaacuugaaaaaguggcaccgagucggugcU | |
| (CD70-7) | | *U*U*U | |
| | Unmodified | GCUUUGGUCCCAUUGGUCGCguuuuagagcuagaaauagcaaguu | 3 |
| | | aaaauaaggcuaguccguuaucaacuugaaaaaguggcaccgagucggugcUUU | |
| | | U | |
| CD70 | Modified | G*C*U*UUGGUCCCAUUGGUCGC | 4 |
| spacer | | | |
| sgRNA | Unmodified | GCUUUGGUCCCAUUGGUCGC | 5 |
| TRAC | Modified | A*G*A*GCAACAGUGCUGUGGCCguuuuagagcuagaaauagcaa | 6 |
| sgRNA | | guuaaaauaaggcuaguccguuaucaacuugaaaaaguggcaccgagucggugcU | |
| (TA-1) | | *U*U*U | |
| | Unmodified | AGAGCAACAGUGCUGUGGCCguuuuagagcuagaaauagcaaguu | 7 |
| | | aaaauaaggcuaguccguuaucaacuugaaaaaguggcaccgagucggugcUUU | |
| | | U | |
| TRAC | Modified | A*G*A*GCAACAGUGCUGUGGCC | 8 |
| spacer | | | |
| sgRNA | Unmodified | AGAGCAACAGUGCUGUGGCC | 9 |
| β2M | Modified | G*C*U*ACUCUCUCUUUCUGGCCguuuuagagcuagaaauagcaag | 10 |
| (B2M-1) | | uuaaaauaaggcuaguccguuaucaacuugaaaaaguggcaccgagucggugcU* | |
| sgRNA | | U*U*U | |
| | Unmodified | GCUACUCUCUCUUUCUGGCCguuuuagagcuagaaauagcaaguua | 11 |
| | | aaauaaggcuaguccguuaucaacuugaaaaaguggcaccgagucggugcUUU | |
| | | U | |
| β2M | Modified | G*C*U*ACUCUCUCUUUCUGGCC | 12 |
| sgRNA | | | |
| spacer | Unmodified | GCUACUCUCUCUUUCUGGCC | 13 |

Target Sequences (PAM)

| | | |
|---|---|---|
| CD70 | GCTTTGGTCCCATTGGTCGC (GGG) | 14 |
| target | | |
| sequence | | |
| with (PAM) | | |
| CD70 | GCTTTGGTCCCATTGGTCGC | 15 |
| target | | |
| sequence | | |
| TRAC | AGAGCAACAGTGCTGTGGCC (TGG) | 16 |
| sgRNA | | |
| TRAC | AGAGCAACAGTGCTGTGGCC | 17 |
| target | | |
| sequence | | |
| with (PAM) | | |
| TRAC | GCTACTCTCTCTTTCTGGCC (TGG) | 18 |
| target | | |
| sequence | | |
| 2M target | GCTACTCTCTCTTTCTGGCC | 19 |
| sequence | | |
| with (PAM) | | |

β2M target sequence

| | | |
|---|---|---|
| sgRNA | nnnnnnnnnnnnnnnnnnnnguuuuagagcuagaaauagcaaguuaaaauaaggcuaguccguuauc | 20 |
| sequence | aacuugaaaaaguggcaccgagucggugcuuuu | |
| sgRNA | nnnnnnnnnnnnnnnnnnnnguuuuagagcuagaaauagcaaguuaaaauaaggcuaguccguuauc | 21 |
| sequence | aacuugaaaaaguggcaccgagucggugc | |
| sgRNA | n(17-30)guuuuagagcuagaaauagcaaguuaaaauaaggcuaguccguuaucaacuugaaa | 22 |
| sequence | aaguggcaccgagucggugcu( 1-8) | |

*indicates a nucleotide with a 2′-O-methyl phosphorothioate modification, “n” refers to the spacer sequence at the 5′ end.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the CD70 target sequence is 5′-GCTTTGGTCCCATTGGTCGC-3′ (SEQ ID NO: 15), then the gRNA spacer sequence is 5′-GCUUUGGUCCCAUUGGUCGC-3′ (SEQ ID NO: 5). In another example, if the TRAC target sequence is 5′-AGAGCAACAGTGCTGTGGCC-3′ (SEQ ID NO: 17), then the gRNA spacer sequence is 5′-AGAGCAACAGUGCUGUGGCC-3′ (SEQ ID NO: 9). In yet another example, if the β2M target sequence is 5′-GCTACTCTCTCTTTCTGGCC-3′ (SEQ ID NO: 19), then the gRNA spacer sequence is 5′-GCUACUCUCUCUUUCUGGCC-3′ (SEQ ID NO: 13). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

A spacer sequence in a gRNA is a sequence (e.g., a 20 nucleotide sequence) that defines the target sequence (e.g., a DNA target sequences, such as a genomic target sequence) of a target gene of interest. An exemplary spacer sequence of a gRNA targeting a CD70 gene is provided in SEQ ID NO: 4. An exemplary spacer sequence of a gRNA targeting a TRAC gene is provided in SEQ ID NO: 8. An exemplary spacer sequence of a gRNA targeting a β2M gene is provided in SEQ ID NO: 12.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of the prior applications are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as a sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of the prior applications are herein incorporated by reference for the purposes and subject matter referenced herein.

In some embodiments, gRNAs targeting the TRAC genomic region create Indels in the TRAC gene comprising at least one nucleotide sequence selected from the sequences in Table 3. In some embodiments, gRNA (e.g., SEQ ID NO: 6) targeting the TRAC genomic region create Indels in the TRAC gene comprising at least one nucleotide sequence selected from the sequences in Table 3.

TABLE 3
Edited TRAC Gene Sequence.

| | Sequence (Deletions indicated by dashes (-); insertions | SEQ ID |
|---|---|---|
| Description | indicated by bold) | NO: |
| TRAC gene edit | AA---------------------GAGCAACAAATCTGACT | 23 |
| TRAC gene edit | AAGAGCAACAGTGCTGT-GCCTGGAGCAACAAATCTGACT | 24 |
| TRAC gene edit | AAGAGCAACAGTG-------CTGGAGCAACAAATCTGACT | 25 |
| TRAC gene edit | AAGAGCAACAGT------GCCTGGAGCAACAAATCTGACT | 26 |
| TRAC gene edit | AAGAGCAACAGTG---------------------CTGACT | 27 |
| TRAC gene edit | AAGAGCAACAGTGCTGTGGGCCTGGAGCAACAAATCTGACT | 28 |
| TRAC gene edit | AAGAGCAACAGTGC--TGGCCTGGAGCAACAAATCTGACT | 29 |
| TRAC gene edit | AAGAGCAACAGTGCTGTGTGCCTGGAGCAACAAATCTGACT | 30 |

In some embodiments, gRNAs targeting the β2M genomic region create Indels in the β2M gene comprising at least one nucleotide sequence selected from the sequences in Table 4. In some embodiments, gRNA (e.g., SEQ ID NO: 10) targeting the β2M genomic region create Indels in the β2M gene comprising at least one nucleotide sequence selected from the sequences in Table 4.

TABLE 4
Edited p2M Gene Sequence.

| | Sequence (Deletions indicated by dashes (-); insertions | SEQ ID |
|---|---|---|
| Description | indicated by bold) | NO: |
| β2M gene-edit | CGTGGCCTTAGCTGTGCTCGCGCTACTCTCTCTTTCT- | 31 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 32 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 33 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAGCCTG | 34 |
| | GAGGCTATCCAGCGTGAGTCTCTCCTACCCTCCCGCT | |
| β2M gene-edit | CGTGGCCTTAGCTGTGCTCGC------------------------- | 35 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGCCTGGA | 36 |
| | GGCTATCCAGCGTGAGTCTCTCCTACCCTCCCGCT | |

In some embodiments, gRNAs targeting the CD70 genomic region create Indels in the CD70 gene comprising at least one nucleotide sequence selected from the sequences in Table 5. In some embodiments, gRNAs targeting the CD70 genomic region create Indels in the CD70 gene comprising at least one nucleotide sequence selected from the sequences in Table 5. In some embodiments, gRNA (e.g., SEQ ID NO: 2) targeting the CD70 genomic region create Indels in the CD70 gene comprising at least one nucleotide sequence selected from the sequences in Table 5.

TABLE 5
Edited CD70 Gene Sequence.

| | Sequence (Deletions indicated by dashes (-); insertions | SEQ ID |
|---|---|---|
| Description | indicated by bold) | NO: |
| CD70 gene-edit | CACACCACGAGGCAGATCACCAAGCCCGCG-- | 37 |
| | CAATGGGACCAAAGCAGCCCGCAGGACG | |
| CD70 gene-edit | CACACCACGAGGCAGATCACCAAGCCCGCGAACCAATGGGACCAAAG | 38 |
| | CAGCCCGCAGGACG | |
| CD70 gene-edit | CACACCACGAGGCAGATC------------ | 39 |
| | ACCAATGGGACCAAAGCAGCCCGCAGGACG | |
| CD70 gene-edit | CACACCACGAGGCAGATCACCAAGCCCGCG- | 40 |
| | CCAATGGGACCAAAGCAGCCCGCAGGACG | |
| CD70 gene-edit | CACACCACGAGGCAGATCACCAAGCCCGC- | 41 |
| | ACCAATGGGACCAAAGCAGCCCGCAGGACG | |
| CD70 gene-edit | CACACCACGAGGCAGATCACCA------------------------- | 42 |
| | AGCCCGCAGGACG | |

### (ii) AAV Vectors for Delivery of CAR Constructs to T Cells

A nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids primarily bind and thus what cells the AAV most efficiently infects. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### III. Treatment of Renal Cell Carcinoma (RCC)

In some aspects, provided herein are methods for treating a human patient having renal cell carcinoma (RCC) using a population of any of the anti-CD70 CAR T cells such as the CTX130 cells as disclosed herein. Such treatment methods may comprise a conditioning regimen (lymphodepleting treatment), which comprises giving one or more doses of one or more lymphodepleting agents to a suitable human patient, and a treatment regimen (anti-CD70 CAR T cell therapy), which comprises administration of the population of anti-CD70 CAR T cells such as the CTX130 cells as disclosed herein to the human patient. When applicable, multiple doses of the anti-CD70 CAR cells may be given to the human patient and a lymphodepletion treatment can be applied to the human patient prior to each dose of the anti-CD70 CAR T cells.

### (i) Patient Population

A human patient may be any human subject for whom diagnosis, treatment, or therapy is desired. A human patient may be of any age. In some embodiments, the human patient is an adult (e.g., a person who is at least 18 years old). In some embodiments, the human patient is a child. In some embodiments, the human patient has a body weight ≥60 kg.

A human patient to be treated by the methods described herein can be a human patient having, suspected of having, or a risk for having renal cell carcinoma (RCC). A subject suspected of having RCC might show one or more symptoms of RCC, e.g., unexplained weight loss, anemia, abdominal pain, blood in the urine, or lumps in the abdomen. A subject at risk for RCC can be a subject having one or more of the risk factors for RCC, e.g., smoking, obesity, high blood pressure, family history of RCC, or genetic conditions such as von Hippel-Lindau disease. A human patient who needs the anti-CD70 CAR T cell (e.g., CTX130 cell) treatment may be identified by routine medical examination, e.g., laboratory tests, biopsy, magnetic resonance imaging (MRI) scans, or ultrasound exams.

Examples of renal cell carcinomas (RCCs) that may be treated using methods described herein include, but are not limited to, clear cell renal carcinomas (ccRCC), papillary renal cell carcinomas (pRCC), and chromophobe renal cell carcinomas (crRCC). These three subtypes account for more than 90% of all RCCs.

In some embodiments, the human patient has unresectable or metastatic RCC. In some embodiments, the human patient has relapsed or refractory RCC. As used herein, “refractory RCC” refers to RCC that does not respond to or becomes resistant to a treatment. As used herein, “relapsed RCC” refers to RCC that returns following a period of complete response. In some embodiments, relapse occurs after the treatment. In other embodiments, relapse occurs during the treatment. A lack of response may be determined by routine medical practice. In some embodiments, the human patient has predominantly clear cell RCC (ccRCC). In some embodiments, the human patient has advanced (e.g., unresectable or metastatic) RCC with clear cell differentiation (e.g., predominantly). In some embodiments, the human patient has relapsed or refractory RCC with clear cell differentiation (e.g., predominantly).

A human patient may be screened to determine whether the patient is eligible to undergo a conditioning regimen (lymphodepleting treatment) and/or a treatment regimen (anti-CD70 CAR T cell therapy). For example, a human patient who is eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, and (f) grade ≥2 acute neurological toxicity. In another example, a human patient who is eligible for a treatment regimen does not show one or more of the following features: (a) active uncontrolled infection, (b) worsening of clinical status compared to the clinical status prior to lymphodepletion treatment, and (c) grade ≥2 acute neurological toxicity (e.g., ICANS).

A human patient may be screened and excluded from the conditioning regimen and/or treatment regimen based on such screening results. For example, a human patient may be excluded from a conditioning regimen and/or a treatment regimen if the patient meets any of the following exclusion criteria: (a) prior treatment with any anti-CD70 targeting agents, (b) prior treatment with any CAR T cells or any other modified T or natural killer (NK) cells, (c) prior anaphylactic reaction to any lymphodepletion treatment or any of the excipients of any treatment regimen, (d) detectable malignant cells from cerebrospinal fluid (CSF) or magnetic resonance imaging (MRI) indicating brain metastases, (e) history or presence of clinically relevant CNS pathology, (f) unstable angina, arrhythmia, or myocardial infarction within 6 months prior to screening, (g) diabetes mellitus with an HBA1c level of 6.5% or 48 mmol/ml, and (h) uncontrolled, acute life-threatening bacterial, viral, or fungal infection.

A human patient subjected to lymphodepletion treatment may be screened for eligibility to receive one or more doses of the anti-CD70 CAR T cells disclosed herein such as the CTX130 cells. For example, a human patient subjected to lymphodepletion treatment that is eligible for an anti-CD70 CAR T cell treatment does not show one or more of the following features: (a) active uncontrolled infection, (b) worsening of clinical status compared to the clinical status prior to lymphodepletion treatment, and (c) grade ≥2 acute neurological toxicity (e.g., ICANS).

Following each dosing of anti-CD70 CAR T cells, a human patient may be monitored for acute toxicities such as cytokine release syndrome (CRS), tumor lysis syndrome (TLS), neurotoxicity (e.g., ICANS), graft versus host disease (GvHD), on target off-tumor toxicity, and/or uncontrolled T cell proliferation. The on target off-tumor toxicity may comprises activity of the population of genetically engineered T cells against activated T lymphocytes, B lymphocytes, dendritic cells, osteoblasts and/or renal tubular-like epithelium. One or more of the following potential toxicity may also be monitored: hypotension, renal insufficiency, hemophagocytic lymphohistiocytosis (HLH), prolonged cytopenias, and/or drug-induced liver injury. After each dose of anti-CD70 CAR T cells, a human patient may be monitored for at least 28 days for development of toxicity.

When a human patient exhibits one or more symptoms of acute toxicity, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CAR T cells.

Alternatively, or in addition to, when a human patient exhibits one or more symptoms of acute toxicity, treatment of the human patient may be terminated. Patient treatment may also be terminated if the patient exhibits one or more signs of an adverse event (AE), e.g., the patient has an abnormal laboratory finding and/or the patient shows signs of disease progression.

Any of the human patients treated using a method disclosed herein may receive subsequent treatment. For example, the human patient is subject to an anti-cytokine therapy. In another example, the human patient is subject to autologous or allogeneic hematopoietic stem cell transplantation after treatment with the population of genetically engineered T cells.

### (ii) Conditioning Regimen (Lymphodepleting Therapy)

Any human patients suitable for the treatment methods disclosed herein may receive a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 20 or 30 mg/m²) per day for 2-4 days (e.g., 3 days) and cyclophosphamide at about 300-600 mg/m² (e.g., 500 mg/m²) per day for 2-4 days (e.g., 3 days). In another example, the human patient receives fludarabine at about 20-30 mg/m² (e.g., 25 mg/m²) per day for 2-4 days (e.g., 3 days) and cyclophosphamide at about 300-600 mg/m² (e.g., 300 or 400 mg/m²) per day for 2-4 days (e.g., 3 days). If needed, the dose of cyclophosphamide may be increased, for example, to up to 1,000 mg/m².

The human patient may then be administered any of the anti-CD70 CAR T cells such as CTX130 cells within a suitable period after the lymphodepleting therapy as disclosed herein. For example, a human patient may be subject to one or more lymphodepleting agent about 2-7 days (e.g., for example, 2, 3, 4, 5, 6, 7 days) before administration of the anti-CD70 CAR+ T cells (e.g., CTX130 cells).

Since the allogeneic anti-CD70 CAR-T cells such as CTX130 cells can be prepared in advance, the lymphodepleting therapy as disclosed herein may be applied to a human patient having RCC within a short time window (e.g., within 2 weeks) after the human patient is identified as suitable for the allogeneic anti-CD70 CAR-T cell therapy disclosed herein.

Methods described herein encompass redosing a human patient with anti-CD70 CAR+ T cells. In such instances, the human patient is subjected to lymphodepletion treatment prior to redosing. For example, a human patient may be subject to a first lymphodepletion treatment and a first dose of CTX130 followed by a second lymphodepletion treatment and a second dose of CTX130. In another example, a human patient may be subject to a first lymphodepletion treatment and a first dose of CTX130, a second lymphodepletion treatment and a second dose of CTX130, and a third lymphodepletion treatment and a third dose of CTX130.

Prior to any of the lymphodepletion steps (e.g., prior to the initial lymphodepletion step or prior to any follow-on lymphodepletion step in association with a re-dosing of the anti-CD70 CAR T cells such as CTX130 cells), a human patient may be screened for one or more features to determine whether the patient is eligible for lymphodepletion treatment. For example, prior to lymphodepletion, a human patient eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, and (f) grade ≥2 acute neurological toxicity (e.g., ICANS).

Following lymphodepletion, a human patient may be screened for one or more features to determine whether the patient is eligible for treatment with anti-CD70 CAR T cells. For example, prior to anti-CD70 CAR T cell treatment and after lymphodepletion treatment, a human patient eligible for anti-CD70 CAR T cells treatment does not show one or more of the following features: (a) active uncontrolled infection, (b) worsening of clinical status compared to the clinical status prior to lymphodepletion treatment, and (c) grade ≥2 acute neurological toxicity (e.g., ICANS).

(iii) Administration of Anti-CD70 CAR T Cells

Aspects of the present disclosure provide methods of treating renal cell carcinoma (RCC) comprising subjecting a human patient to lymphodepletion treatment and administering to the human patient a dose of a population of genetically engineered T cells described herein (e.g., CTX130 cells).

Administering anti-CD70 CAR T cells may include placement (e.g., transplantation) of a genetically engineered T cell population into a human patient by a method or route that results in at least partial localization of the genetically engineered T cell population at a desired site, such as a tumor site, such that a desired effect(s) can be produced. The genetically engineered T cell population can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of the genetically engineered T cell population can be administered via a systemic route of administration, such as an intraperitoneal or intravenous route.

In some embodiments, the genetically engineered T cell population is administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

An effective amount refers to the amount of a genetically engineered T cell population needed to prevent or alleviate at least one or more signs or symptoms of a medical condition (e.g., cancer such as renal cell carcinoma), and relates to a sufficient amount of a genetically engineered T cell population to provide the desired effect, e.g., to treat a subject having a medical condition (e.g., renal cell carcinoma). An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

An effective amount of a genetically engineered T cell population may comprise about 1×10⁶ cells to about 1×10⁹ CAR+ cells, e.g., about 3.0×10⁷ cells to about 1×10⁹ cells that express an anti-CD70 CAR (CAR⁺ cells), for example, CAR⁺ CTX130 cells. In some embodiments, an effective amount of a genetically engineered T cell population may comprise about 3.0×10⁷ CAR+ cells to about 9×10⁸ cells that express an anti-CD70 CAR, for example, CAR⁺ CTX130 cells. In some embodiments, an effective amount of a genetically engineered T cell population may comprise at least 3.0×10⁸ CAR⁺ CTX130 cells, at least 4×10⁸ CAR⁺ CTX130 cells, at least 4.5×10⁸ CAR⁺ CTX130 cells, at least 5×10⁸ CAR⁺ CTX130 cells, at least 5.5×10⁸ CAR⁺ CTX130 cells, at least 6×10⁸ CAR⁺ CTX130 cells, at least 6.5×10⁸ CAR⁺ CTX130 cells, at least 7×10⁸ CAR⁺ CTX130 cells, at least 7.5×10⁸ CAR⁺ CTX130 cells, at least 8×10⁸ CAR⁺ CTX130 cells, at least 8.5×10⁸ CAR⁺ CTX130 cells, or at least 9×10⁸ CAR⁺ CTX130 cells. In some examples, the amount of the CAR⁺ CTX130 cells may not exceed 1×10⁹ cells.

In some embodiments, an effective amount of the genetically engineered T cell population as disclosed herein (e.g., the CTX130 cells) may range from about 3.0×10⁷ to about 3×10⁸ CAR⁺ T cells, for example, about 1×10⁷ to about 1×10⁸ CAR⁺ T cells or about 1×10⁸ to about 3×10⁸ CAR⁺ T cells. In some embodiments, an effective amount of the genetically engineered T cell population as disclosed herein (e.g., the CTX130 cells) may range from about 1.5×10⁸ to about 3×10⁸ CAR⁺ T cells.

In some embodiments, an effective amount of the genetically engineered T cell population as disclosed herein (e.g., the CTX130 cells) may range from about 3.0×10⁸ to about 9×10⁸ CAR⁺ T cells, for example, about 3.5×10⁸ to about 6×10⁸ CAR⁺ T cells or about 3.5×10⁸ to about 4.5×10⁸ CAR⁺ T cells. In some embodiments, an effective amount of the genetically engineered T cell population as disclosed herein (e.g., the CTX130 cells) may range from about 4.5×10⁸ to about 9×10⁸ CAR⁺ T cells. In some embodiments, an effective amount of the genetically engineered T cell population as disclosed herein (e.g., the CTX130 cells) may range from about 4.5×10⁸ to about 6×10⁸ CAR⁺ T cells. In some embodiments, an effective amount of the genetically engineered T cell population as disclosed herein (e.g., the CTX130 cells) may range from about 6×10⁸ to about 9×10⁸ CAR⁺ T cells. In some embodiments, an effective amount of the genetically engineered T cell population as disclosed herein (e.g., the CTX130 cells) may range from about 7.5×10⁸ to about 9×10⁸ CAR⁺ T cells.

In specific examples, an effective amount of the genetically engineered T cell population as disclosed herein (e.g., the CTX130 cells) may comprise about 3.0×10⁸ CAR⁺ T cells. For example, an effective amount of the genetically engineered T cell population as disclosed herein (e.g., the CTX130 cells) may comprise about 4.5×10⁸ CAR⁺ T cells. In other examples, an effective amount of the genetically engineered T cell population as disclosed herein (e.g., the CTX130 cells) may comprise about 6×10⁸ CAR⁺ T cells. In some examples, an effective amount of the genetically engineered T cell population as disclosed herein (e.g., the CTX130 cells) may comprise about 7.5×10⁸ CAR⁺ T cells. In yet other examples, an effective amount of the genetically engineered T cell population as disclosed herein (e.g., the CTX130 cells) may comprise about 9×10⁸ CAR⁺ T cells.

In some embodiments, an effective amount of the genetically engineered T cell population as disclosed herein (e.g., the CTX130 cells) may range from about 3×10⁸ to about 9×10⁸ CAR⁺ T cells. In some embodiments, an effective amount of the genetically engineered T cell population as disclosed herein (e.g., the CTX130 cells) may range from about 3×10⁸ to about 7.5×10⁸ CAR⁺ T cells. In some embodiments, an effective amount of the genetically engineered T cell population as disclosed herein (e.g., the CTX130 cells) may range from about 3×10⁸ to about 6×10⁸ CAR⁺ T cells. In some embodiments, an effective amount of the genetically engineered T cell population as disclosed herein (e.g., the CTX130 cells) may range from about 3×10⁸ to about 4.5×10⁸ CAR⁺ T cells.

In some embodiments, an effective amount of a genetically engineered T cell population may comprise a dose of the genetically engineered T cell population, e.g., a dose comprising about 3.0×10⁸ CAR⁺ CTX130 cells to about 9×10⁸ CAR⁺ CTX130 cells, e.g., any dose or range of doses disclosed herein. In some examples, the effective amount is 4.5×10⁶ CAR⁺ CTX130 cells. In some examples, the effective amount is 6×10⁸ CAR⁺ CTX130 cells. In some examples, the effective amount is 7.5×10⁸ CAR⁺ CTX130 cells. In some examples, the effective amount is 9×10⁸ CAR⁺ CTX130 cells.

In some examples, a patient having advanced (e.g., unresectable or metastatic) RCC or relapsed/refractory RCC may be given a suitable dose of CTX130 cells, for example, about 3×10⁷ to about 6×10⁸ CAR⁺ CTX130 cells. Such an RCC patient may be administered about 3×10⁷ CAR⁺ CTX130 cells. Alternatively, the RCC patient may be administered about 1×10⁸ CAR⁺ CTX130 cells. In another example, the RCC patient may be administered about 3×10⁸ CAR⁺ CTX130 cells. In another example, the RCC patient may be administered about 4.5×10⁸ CAR⁺ CTX130 cells. In another example, the RCC patient may be administered about 6×10⁸ CAR⁺ CTX130 cells. In another example, the RCC patient may be administered about 7.5×10⁸ CAR⁺ CTX130 cells. In another example, the RCC patient may be administered about 9×10⁸ CAR⁺ CTX130 cells.

In some examples, a patient having advanced (e.g., unresectable or metastatic) RCC or relapsed/refractory RCC may be given a suitable dose of CTX130 cells, for example, about 9×10⁹ to about 1×10⁹ CAR⁺ CTX130 cells. Such an RCC patient may be administered about 9×10⁹ CAR⁺ CTX130 cells. Alternatively, the RCC patient may be administered about 1.0×10⁹ CAR⁺ CTX130 cells.

In some embodiments, a suitable dose of CTX130 cells administered from one or more vials of the pharmaceutical composition, each comprising about 1.5×10⁸ CAR+ CTX130 cells. In some embodiments, a suitable dose of CTX130 cells is administered from one or more vials of the pharmaceutical composition, each comprising about 3×10⁸ CAR+ CTX130 cells. In some embodiments, a suitable dose of CTX130 cells administered to a subject is one or more folds of 1.5×10⁸ CAR+ CTX130 cells, for example, 1-fold, 2-fold, 3-fold, 4-fold, 5-fold, or 6-fold of CAR+ CTX130 cells. In some embodiments a suitable dose of CTX130 cells is administered from one or more full or partial vials of the pharmaceutical composition.

The efficacy of anti-CD70 CAR T cell therapy described herein can be determined by the skilled clinician. An anti-CD70 CAR T cell therapy is considered “effective”, if any one or all of the signs or symptoms of, as but one example, levels of CD70 are altered in a beneficial manner (e.g., decreased by at least 10%), or other clinically accepted symptoms or markers of renal cell carcinoma are improved or ameliorated. Efficacy can also be measured by failure of a subject to worsen as assessed by hospitalization or need for medical interventions (e.g., progression of the renal cell carcinoma is halted or at least slowed). Methods of measuring these indicators are known to those of skill in the art and/or described herein. Treatment includes any treatment of a renal cell carcinoma in a human patient and includes: (1) inhibiting the disease, e.g., arresting, or slowing the progression of symptoms; or (2) relieving the disease, e.g., causing regression of symptoms; and (3) preventing or reducing the likelihood of the development of symptoms.

Treatment methods described herein encompass repeating lymphodepletion and redosing of anti-CD70 CAR T cells. Prior to each redosing of anti-CD70 CAR T cells, the patient is subjected to another lymphodepletion treatment. The doses of anti-CD70 CAR T cells may be the same for the first, second, and third doses. For example, each of the first, second, and third doses is 1×10⁶ CAR+ cells, 1×10⁷ CAR+ cells, 3×10⁷ CAR+ cells, 1×10⁸ CAR+ cells, 1.5×10⁸ CAR+ cells, 4.5×10⁸ CAR⁺ cells, 6×10⁸ CAR⁺ cells, 7.5×10⁸ CAR⁺ cells, 9.8×10⁸, or 1×10⁹ CAR+ cells. In other instances, the doses of anti-CD70 CAR T cells may increase in number of CAR+ cells as the number of doses increases. For example, the first dose is 1×10⁶ CAR+ cells, the second dose is 1×10⁷ CAR+ cells, and the third dose is 1×10⁸ CAR+ cells. Alternatively, the first dose of CAR+ cells is lower than the second and/or third dose of CAR+ cells, e.g., the first dose is 1×10⁶ CAR+ cells and the second and the third doses are 1×10⁸ CAR+ cells. In some examples, the dose of anti-CD70 CAR T cells may increase by 1.5×10⁸ CAR+ cells for each subsequent dose.

Patients may be assessed for redosing following each administration of anti-CD70 CAR T cells. For example, following a first dose of anti-CD70 CAR T cells, a human patient may be eligible for receiving a second dose of anti-CD70 CAR T cells if the patient does not show one or more of the following: (a) dose-limiting toxicity (DLT), (b) grade 4 CRS that does not resolve to grade 2 within 72 hours, (c) grade >1 GvHD, (d) grade ≥3 neurotoxicity, (e) active infection, (f) hemodynamically unstable, and (g) organ dysfunction. In another example, following a second dose of anti-CD70 CAR T cells, a human patient may be eligible for receiving a third dose of CTX130 if that patient does not show one or more of the following: (a) dose-limiting toxicity (DLT), (b) grade 4 CRS that does not resolve to grade 2 within 72 hours, (c) grade >1 GvHD, (d) grade ≥3 neurotoxicity, (e) active infection, (f) hemodynamically unstable, and (g) organ dysfunction.

In some embodiments, a human patient as disclosed herein may be given multiple doses of the anti-CD70 CART cells (e.g., the CTX130 cells as disclosed herein), i.e., re-dosing. The human patient may be given up to three doses in total (i.e., re-dosing for no more than 2 times). The interval between two consecutive doses may be about 8 weeks to about 2 years. In some examples, a human patient may be re-dosed if the patient achieved a partial response (PR) or complete response (CR) after a first dose (or a second dose) and subsequently progressed within 2 years of last dose. In other examples, a human patient may be re-dosed when the patient achieved PR (but not CR) or stable disease (SD) after the most recent dose. See also Example 9 below.

Redosing of anti-CD70 CAR T cells such as CTX130 cells may take place about 8 weeks to about 2 years after the first dose of the anti-CD70 CAR T cells. For example, redosing of anti-CD70 CAR T cells may take place about 8-10 weeks after the first dose of anti-CD70 CAR T cells. In other examples, redosing of anti-CD70 CAR T cells may take place about 14-18 weeks after the first dose of the anti-CD70 CAR T cells. When a patient is administered two doses, the second dose may be administered 8 weeks to two years (e.g., 8-10 weeks or 14-18 weeks) after the preceding dose. In some examples, a patient can be administered three doses. The third dose may be administered 14-18 weeks after the first dose, and the second dose may be administered 6-10 weeks after the first dose. In some instances, the interval between two consecutive doses may be about 6-10 weeks.

Following each dosing of anti-CD70 CAR T cells, a human patient may be monitored for acute toxicities such as cytokine release syndrome (CRS), tumor lysis syndrome (TLS), neurotoxicity (e.g., ICANS), graft versus host disease (GvHD), on target off-tumor toxicity, and/or uncontrolled T cell proliferation. The on target off-tumor toxicity may comprises activity of the population of genetically engineered T cells against activated T lymphocytes, B lymphocytes, dendritic cells, osteoblasts and/or renal tubular-like epithelium. One or more of the following potential toxicity may also be monitored: hypotension, renal insufficiency, hemophagocytic lymphohistiocytosis (HLH), prolonged cytopenias, and/or drug-induced liver injury. After each dose of anti-CD70 CAR T cells, a human patient may be monitored for at least 28 days for development of toxicity. If development of toxicity is observed, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CAR T cells.

Anti-CD70 CAR T cell treatment methods described herein may be used on a human patient that has undergone a prior anti-cancer therapy. For example, anti-CD70 CAR T cells as described herein may be administered to a patient that has been previously treated with a checkpoint inhibitor, a tyrosine kinase inhibitor, a vascular endothelial growth factor (VEGF), or a combination thereof.

Anti-CD70 CAR T cells treatment methods described herein may also be used in combination therapies. For example, anti-CD70 CAR T cells treatment methods described herein may be co-used with other therapeutic agents, for treating renal cell carcinoma, or for enhancing efficacy of the genetically engineered T cell population and/or reducing side effects of the genetically engineered T cell population.

### IV. Kit for Treating Renal Cell Carcinoma

The present disclosure also provides kits for use of a population of anti-CD70 CAR T cells such as CTX130 cells as described herein in methods for treating renal cell carcinoma (RCC). Such kits may include one or more containers comprising a first pharmaceutical composition that comprises one or more lymphodepleting agents, and a second pharmaceutical composition that comprises any nucleic acid or population of genetically engineered T cells (e.g., those described herein), and a pharmaceutically acceptable carrier.

In some embodiments, the kit can comprise instructions for use in any of the methods described herein. The included instructions can comprise a description of administration of the first and/or second pharmaceutical compositions to a subject to achieve the intended activity in a human patient. The kit may further comprise a description of selecting a human patient suitable for treatment based on identifying whether the human patient is in need of the treatment. In some embodiments, the instructions comprise a description of administering the first and second pharmaceutical compositions to a human patient who is in need of the treatment.

The instructions relating to the use of a population of anti-CD70 CAR T cells such as CTX130 cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a renal cell carcinoma in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-CD70 CAR-T cells such as the CTX130 cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986»; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Example 1: Generation of T Cells with Multiple Gene Knockouts

This example describes the use of CRISPR/Cas9 gene editing technology to produce human T cells that lack expression of two or three genes simultaneously. Specifically, the T cell receptor (TCR) gene (gene edited in the TCR Alpha Constant (TRAC) region), the β2-microglobulin (β2M) gene, and the Cluster of Differentiation 70 (CD70) gene were edited by CRISPR/Cas9 gene editing to produce T cells deficient in two or more of the listed genes. The following abbreviations are used in for brevity and clarity:

2×KO: TRAC⁻/β2M⁻

3×KO (CD70): TRAC⁻/β2M⁻/CD70⁻

Activated primary human T cells were electroporated with Cas9:gRNA RNP complexes. The nucleofection mix contained the Nucleofector™ Solution, 5×10⁶ cells, 1 μM Cas9, and 5 μM gRNA (as described in Hendel et al., Nat Biotechnol. 2015; 33(9):985-989, PMID: 26121415). For the generation of double knockout T cells (2×KO), the cells were electroporated with two different RNP complexes, each containing Cas9 protein and one of the following sgRNAs: TRAC (SEQ ID NO: 6) and β2M (SEQ ID NO: 10) at the concentrations indicated above. For the generation of triple knockout T cells (3×KO), the cells were electroporated with three different RNP complexes, each RNA complex containing Cas protein and one of the following sgRNAs: (a) TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). See also sequences in Table 6.

TABLE 6
gRNA Sequences/Target Sequences.

| Name | Unmodified Sequence | Modified Sequence |
|---|---|---|
| TRAC sgRNA | AGAGCAACAGUGCUGUG | A*G*A*GCAACAGUGCUG |
| | GCCguuuuagagcuagaaauagcaa | UGGCCguuuuagagcuagaaauag |
| | guuaaaauaaggcuaguccguuauca | caaguuaaaauaaggcuaguccguua |
| | acuugaaaaaguggcaccgagucggu | ucaacuugaaaaaguggcaccgagucg |
| | gcUUUU | gugcU*U*U*U (SEQ ID NO: |
| | (SEQ ID NO: 7) | 6) |
| TRAC sgRNA spacer | AGAGCAACAGUGCUGUG | A*G*A*GCAACAGUGCUG |
| | GCC(SEQ ID NO: 9) | UGGCC(SEQ ID NO: 8) |
| β2M sgRNA | GCUACUCUCUCUUUCUGG | G*C*U*ACUCUCUCUUUC |
| | CCguuuuagagcuagaaauagcaag | UGGCCguuuuagagcuagaaauag |
| | uuaaaauaaggcuaguccguuaucaac | caaguuaaaauaaggcuaguccguua |
| | uugaaaaaguggcaccgagucggugc | ucaacuugaaaaaguggcaccgagucg |
| | UUUU | gugcU*U*U*U |
| | (SEQ ID NO: 11) | (SEQ ID NO: 10) |
| β2M sgRNA spacer | GCUACUCUCUCUUUCUGG | G*C*U*ACUCUCUCUUUC |
| | CC(SEQ ID NO: 13) | UGGCC(SEQ ID NO: 12) |
| CD70 sgRNA; also referred | GCUUUGGUCCCAUUGGU | G*C*U*UUGGUCCCAUUG |
| to as: T7 | CGCguuuuagagcuagaaauagcaa | GUCGCguuuuagagcuagaaauag |
| | guuaaaauaaggcuaguccguuauca | caaguuaaaauaaggcuaguccguua |
| | acuugaaaaaguggcaccgagucggu | ucaacuugaaaaaguggcaccgagucg |
| | gcUUUU | gugcU*U*U*U (SEQ ID NO: |
| | (SEQ ID NO: 3) | 2) |
| CD70 sgRNA spacer; also | GCUUUGGUCCCAUUGGU | G*C*U*UUGGUCCCAUUG |
| referred to as: T7 | CGC(SEQ ID NO: 5) | GUCGC(SEQ ID NO: 4) |
| CD70 sgRNA; also referred | GCCCGCAGGACGCACCCA | G*C*C*CGCAGGACGCACC |
| to as: T8 | UAguuuuagagcuagaaauagcaag | CAUAguuuuagagcuagaaauagc |
| | uuaaaauaaggcuaguccguuaucaac | aaguuaaaauaaggcuaguccguuau |
| | uugaaaaaguggcaccgagucggugc | caacuugaaaaaguggcaccgagucgg |
| | UUUU | ugcU*U*U*U (SEQ ID NO: |
| | (SEQ ID NO: 67) | 66) |
| CD70 sgRNA spacer; also | GCCCGCAGGACGCACCCA | G*C*C*CGCAGGACGCACC |
| referred to as: T8 | UA (SEQ ID NO: 69) | CAUA (SEQ ID NO: 68) |

About one (1) week post electroporation, cells were either left untreated or treated with phorbol myristate acetate (PMA)/ionomycin overnight. The next day cells were processed for flow cytometry (see, e.g., Kalaitzidis D et al., J Clin Invest 2017; 127(4): 1405-1413) to assess TRAC, β2M, and CD70 expression levels at the cell surface of the edited cell population. The following primary antibodies were used (Table 7):

TABLE 7
Antibodies.

| Antibody | Clone | Fluor | Catalogue # | Dilution | For 1 |
|---|---|---|---|---|---|
| TCR | BW242/412 | PE | 130-091-236 | 1:100 | 1 μL |
| | | | (Miltenyi) | | |
| β2M | 2M2 | PE-Cy7 | 316318 | 1:100 | 1 μL |
| | | | (Biolegend) | | |
| CD70 | 113-16 | FITC | 355105 | 1:100 | 1 μL |
| | | | (Biolegend) | | |

Table 8 shows highly efficient multiple gene editing. For the triple knockout cells, 80% of viable cells lacked expression of TCR, β2M, and CD70 (Table 8).

TABLE 8
% of viable cells lacking expression in 3KO cell populations.

| | TRAC KO | β2M KO | CD70 KO | 3KO |
|---|---|---|---|---|
| 3KO (CD70) | 99% | 79% | 99% | 80% |

To assess whether triple gene editing in T cells affects cell expansion, cell numbers were enumerated among double and triple gene edited T cells (unedited T cells were used as a control) over a two week period of post editing. 5×10⁶ cells were generated and plated for each genotype of T cells.

Cell proliferation (expansion) continued over the post-electroporation window test. Similar cell proliferation was observed among the double β2M−/TRAC−) and triple β2M−/TRAC−/CD70−), knockout T cells, as indicated by the number of viable cells (data not shown). These data suggest that multiple gene editing does not impact T cell health as measured by T cell proliferation.

### Example 2: Generation of Anti-CD70 CAR T Cells with Multiple Knockouts

This example describes the production of allogeneic human T cells that lack expression of the TCR gene, β2M gene, and/or CD70 gene, and express a chimeric antigen receptor (CAR) targeting CD70. These cells are designated TCR⁻/β2M⁻/CD70⁻/anti-CD70 CAR⁺ or 3×KO (CD70) CD70 CAR⁺.

A recombinant adeno-associated adenoviral vector, serotype 6 (AAV6) (MOI 50, 000) comprising the nucleotide sequence of SEQ ID NO: 43 (comprising the donor template in SEQ ID NO: 44, encoding anti-CD70 CAR comprising the amino acid sequence of SEQ ID NO: 46) was delivered with Cas9:sgRNA RNPs (1 μM Cas9, 5 μM gRNA) to activated allogeneic human T cells. The following sgRNAs were used: TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). About one (1) week post electroporation, cells were processed for flow cytometry to assess TRAC, β2M, and CD70, expression levels at the cell surface of the edited cell population. The following primary antibodies were used (Table 9):

TABLE 9
Antibodies.

| Antibody | Clone | Fluor | Catalogue # | Dilution |
|---|---|---|---|---|
| TCR | BW242/412 | PE | 130-091-236 (Miltenyi) | 1:100 |
| β2M | 2M2 | PE-Cy7 | 316318 (Biolegend) | 1:100 |
| CD70 | 113-16 | FITC | 355105 (Biolegend) | 1:100 |

T cell Proportion Assay. The proportions of CD4+ and CD8+ cells were then assessed in the edited T cell populations by flow cytometry using the following antibodies (Table 10):

TABLE 10
Antibodies.

| Antibody | Clone | Fluor | Catalogue # | Dilution |
|---|---|---|---|---|
| CD4 | RPA-T4 | BV510 | 300545 (Biolegend) | 1:100 |
| CD8 | SK1 | BV605 | 344741 (Biolegend) | 1:100 |

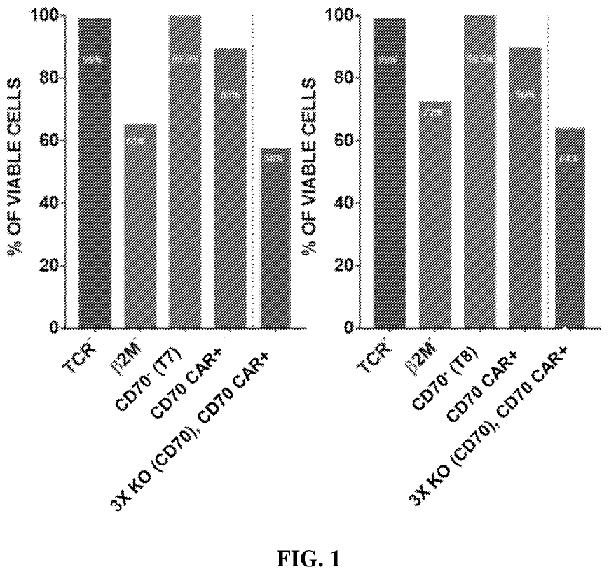

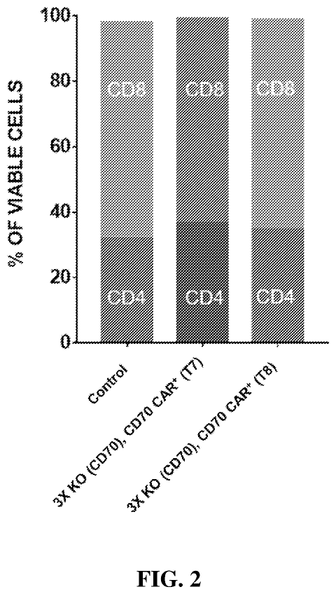

High efficiency gene editing and CAR expression was achieved in the edited anti-CD70 CAR T cell populations. In addition, editing did not adversely alter CD4/CD8 T cell populations. FIG. 1 shows highly efficient gene editing and anti-CD70 CAR expression in the triple knockout CAR T cell. More than 55% of viable cells lacked expression of TCR, β2M, and CD70, and also expressed the anti-CD70 CAR. FIG. 2 shows that normal proportions of CD4/CD8 T cell subsets were maintained in the TRAC−/β2M−/CD70−/anti-CD70 CAR+ cells, suggesting that these multiple gene edits do not affect T cell biology as measured by the proportion of CD4/CD8 T cell subsets.

### Example 3: Effect of CD70 KO on Cell Proliferation of Anti-CD70 CAR T Cells In Vitro

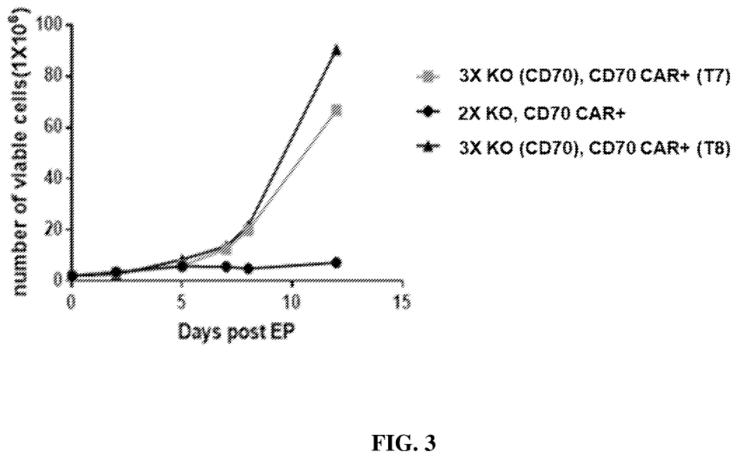

To further assess the impact of disrupting the CD70 gene in CAR T cells, anti-CD70 CAR T cells were generated as described in Example 2. Specifically, 3×KO (TRAC−/β2M−/CD70−) anti-CD70 CAR T cells were generated using two different gRNAs (T7 (SEQ ID NO: 2 and T8 (SEQ ID NO: 66)). After electroporation, cell expansion was assessed by enumerating double or triple gene edited T cells over a two week period of post editing. 5×10⁶ cells were generated and plated for each genotype of T cells. Proliferation was determined by counting number of viable cells. FIG. 3 shows that triple knockout TRAC⁻/β2M⁻/CD70⁻/anti-CD70 CAR⁺ T cells generated with either T7 or T8 gRNAs exhibited greater cell expansion relative to double knockout TRAC⁻/β2M⁻/anti-CD70 CAR⁺ T cells. These data suggest that knocking-out the CD70 gene gives a cell proliferation advantage to anti-CD70 CAR+ T cells.

### Example 4: Cell Killing Function of Anti-CD70 CAR T Cells with CD70 Knock-Out

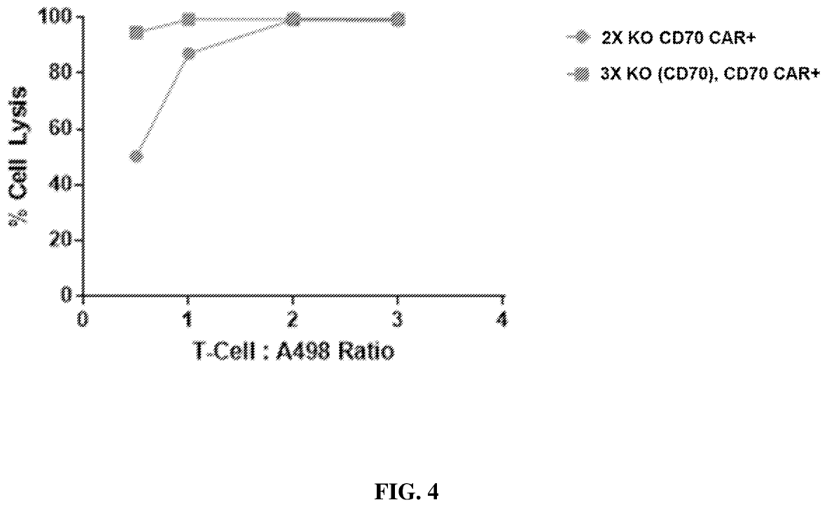

A cell killing assay was used to assess the ability of the TRAC⁻/β2M⁻/CD70⁻/anti-CD70 CAR⁺ T cells and TRAC⁻/β2M⁻/anti-CD70 CAR⁺ T cells to kill a CD70⁺ adherent renal cell carcinoma (RCC)-derived cell line (A498 cells). Adherent cells were seeded in 96-well plates at 50,000 cells per well and left overnight at 37° C. The next day edited anti-CD70 CAR T cells were added to the wells containing target cells at the indicated ratios. After the indicated incubation period, CAR T cells were removed from the culture by aspiration and 100 μL Cell titer-Glo (Promega) was added to each well of the plate to assess the number of remaining viable cells. The amount of light emitted per well was then quantified using a plate reader. The cells exhibited potent cell killing of RCC-derived cells following 24-hour co-incubation (FIG. 4). The anti-CD70 CAR T cells demonstrated higher potency when CD70 was knocked out, which is clearly visible at low T cell: A498 ratios (1:1 and 0.5:1) where cell lysis remains above 90% for TRAC⁻/β2M⁻/CD70⁻/anti-CD70 CAR⁺ T cells, while cells lysis drops below 90% for the TRAC⁻/β2M⁻/anti-CD70 CAR⁺ T cells. This suggests that knocking-out the CD70 gene gives a higher cell kill potency to anti-CD70 CAR+ T cells.

### Example 5: Knockout of CD70 Maintained Anti-CD70 CAR⁺ T Cell Killing Upon Serial Rechallenge

The anti-CD70 CAR⁺ T cells generated above were serially rechallenged with CD70+ kidney cancer cell line, A498, and evaluated for their ability to kill the CD70+ kidney cancer cell line A498.

A498 cells were plated in a T25 flask and mixed at a ratio of 2:1 (T-cell to A498) with 10×10⁶ anti-CD70 CAR⁺ T cells containing either two (TRAC⁻/β2M⁻) or three (TRAC⁻/β2M⁻/CD70⁻)) gRNA edits. Anti-CD70 CAR+ T cells with three edits are also referred to as CTX130.

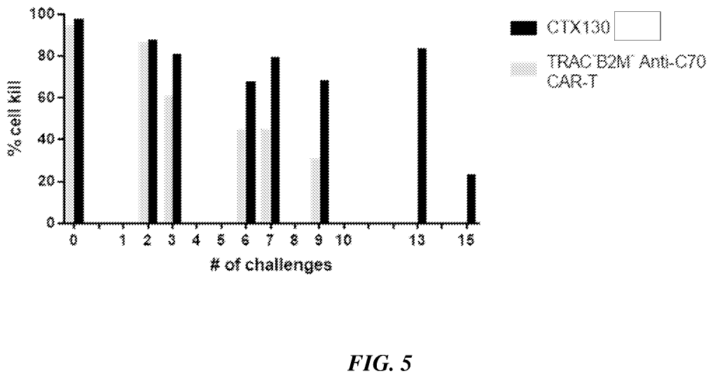

Two or three days after each challenge, cells were counted, washed, resuspended in fresh T cell media, and re-challenged the next day with the same ratio of two anti-CD70 CAR⁺ T cell per one A498 cell (2:1, CAR⁺ T:target). Challenging of anti-CD70 CAR⁺ T cells with CD70+ A498 cells was repeated 13 times. Three to four days following each exposure to A498 cells (and prior to the next rechallenge), aliquots of the culture were taken and analyzed for the ability of the CAR T Cells to kill A498 target cells at a ratio of 2:1 (CAR T cell: Target cell). Cell kill was measured using Cell titer-glo (Promega). Prior to the first challenge with A498, anti-CD70 CAR+ T cells with 2×KO (TRAC⁻/β2M⁻) and 3×KO (TRAC⁻/β2M/CD70⁻), each exhibited a target cell killing of A498 cells approaching 100%. By challenge nine however, the 2×KO (TRAC⁻/β2M⁻) anti-CD70 CAR⁺ T cells induced target cell killing of A498 cells below 40%, while 3×KO (TRAC⁻/β2M⁻/CD70⁻) anti-CD70 CAR⁺ T cells exhibited target cell killing above 60% (FIG. 5). The target cell killing for 3×KO (TRAC⁻/β2M⁻/CD70⁻) anti-CD70 CAR⁺ T cells remained above 60% even following 13 re-challenges with A498 cells, demonstrating that these CAR+ T cells were resistant to exhaustion.

### Example 6: Measurement of Cytokine Secretion by Anti-CD70 CAR+ T Cells (CTX130) in the Presence of CD70+ Cells

The objective of this study was to assess the ability of CTX130 to secrete effector cytokines in the presence of CD70 expressing cells.

Target cancer cell lines (A498, ACHN & MCF7) were obtained from ATCC (HTB-44, CRL-1611 & HTB-22). Expression of CD70 on target cell lines was evaluated. In brief, CTX130 or control T cells (unedited T cells) were co-cultured with target cell lines in U-bottom 96-well plates at varying ratios of T cells to target cells from 0.125:1 up to 4:1. The cells were cultured in total of 200 μL of target cell media for 24 hours, as described in each experiment. Assay was performed in media which did not contain addition of IL-2 and IL-7 to evaluate T cell activation in the absence of supplemental cytokines.

The ability of CTX130 or control T cells (unedited T cells with no anti-CD70 CAR expression) to specifically secrete the effector cytokines interferon-γ (INFγ) and interleukin-2 (IL-2) following co-culture with CD70 positive or CD70 negative target cells was assessed using a Luminex based MILLIPLEX assay as described herein. A498 and ACHN cell lines were used as CD70⁺ target lines, and the MCF7 cell line was used as a CD70− target line. Since the assay was performed in conjunction with the cytotoxicity assay, the protocol was as follows: Target cells were seeded (50,000 target cells per 96-well plate) overnight and then co-cultured with CTX130 or control T cells at varying ratios (0.125:1, 0.25:1, 0.5:1, 1:1, 2:1 and to 4:1 T cells to target cells). Twenty-four hours later, plates were centrifuged, supernatant was collected and stored at −80° C. until further processing. IL-2 and IFNγ were quantified as follows: the MILLIPLEX® kit (Millipore, catalog #HCYTOMAG-60K) was used to quantify IFN-γ and IL-2 secretion using magnetic microspheres, HCYIFNG-MAG (Millipore, catalog #HCYIFNG-MAG) and HIL2-MAG (Millipore, catalog #HIL2-MAG), respectively. The assay was conducted following manufacturer's protocol. In short, MILLIPLEX® standard and quality control (QC) samples were reconstituted, and serial dilutions of the working standards from 10,000 pg/mL to 3.2 pg/mL were prepared. MILLIPLEX® standards, QCs and cell supernatants were added to each plate, and assay media was used to dilute the supernatants. All samples were incubated with HCYIFNG-MAG and HIL2-MAG beads for 2 hours. After incubation, the plate was washed using an automated magnetic plate washer. Human cytokine/chemokine detection antibody solution was added to each well and incubated for 1 hour followed by incubation with Streptavidin-Phycoerythrin for 30 minutes. The plate was subsequently washed, samples were resuspended with 150 μL Sheath Fluid, and agitated on a plate shaker for 5 minutes. The samples were read using the Luminex® 100/200™ instrument with xPONENT® software and data acquisition and analysis was completed using MILLIPLEX® Analyst software. The Median Fluorescent Intensity (MFI) data is automatically analyzed using a 5-parameter logistic curve-fitting method for calculating the cytokine concentration measured in the unknown samples.

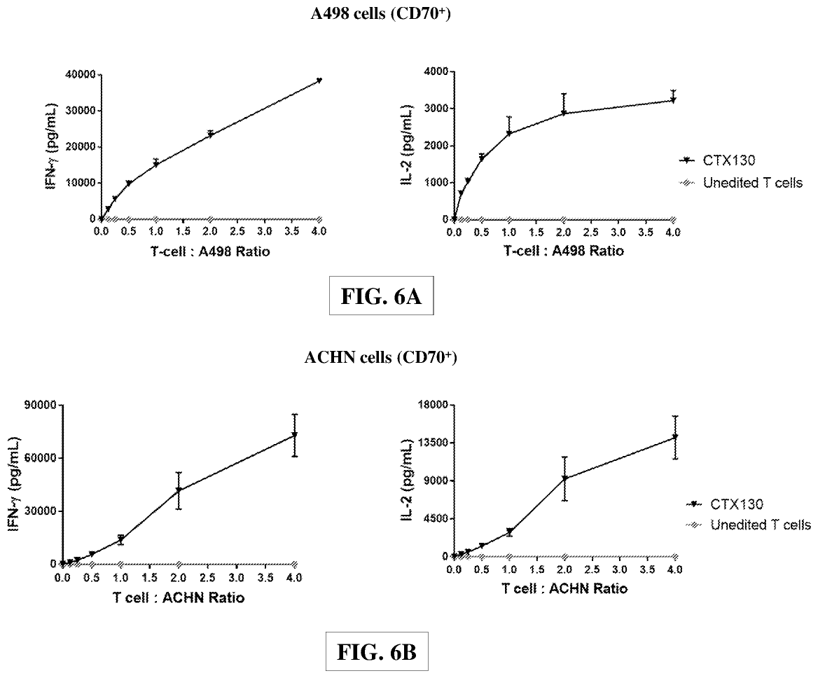

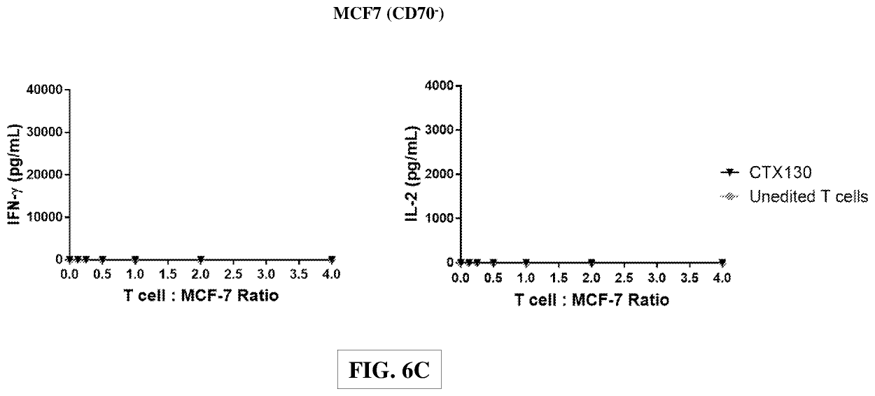

To determine if CTX130 secrete cytokines in the presence of CD70− positive and CD70− negative cells, the development lot 01 was co-cultured for 24 hours with A498, ACHN or MCF7 cells. CTX130 cells secreted both IFNγ and IL-2 following co-culture with CD70+ cells (A498 and ACHN), but not when co-cultured with CD70 negative cells (MCF7) (FIGS. 6A-6C, Tables 11-16). Unedited control T cells showed no specific effector cytokine secretion on the cell lines tested.

TABLE 11
Secretion of IFNγ by CTX130 cells in the presence of CD70+
cell line A498.

| T cell: | IFNγ (pg/mL) | | | | | |
|---|---|---|---|---|---|---|
| A498 ratio | CTX130 | | | Unedited T cells | | |
| 0 | 6.54* | 6.54* | 7.77 | 6.54* | 7.14 | 6.54* |
| 0.125 | 2592.57 | 2466.99 | 3213 | 6.54 | 6.54* | 6.54* |
| 0.25 | 5991 | 5592 | 5196 | 9.75 | 7.14 | 8.4 |
| 0.5 | 10713 | 9300 | 9354 | 7.14 | 6.54* | 9.75 |
| 1 | 16830 | 14514 | 13752 | 6.54* | 6.54 | 8.4 |
| 2 | 24645 | 22809 | 22053 | 8.4 | 14.01 | 15.54 |
| 4 | 38364 | 38364 | 38238 | 11.82 | 10.41 | 17.1 |

Samples marked with an asterisks (*) indicate the value was below the LoD (which was 6.54 pg/ml).

TABLE 12
Secretion of IL-2 by CTX130 cells in the presence of CD70+
cell line A498.

| T cell: | IL-2 (pg/mL) | | | | | |
|---|---|---|---|---|---|---|
| A498 ratio | CTX130 | | | Unedited T cells | | |
| 0 | 6.15* | 6.15* | 6.15* | 6.15* | 6.15* | 6.15* |
| 0.125 | 733.14 | 668.61 | 728.22 | 6.15* | 6.15* | 6.15* |
| 0.25 | 916.05 | 1056.24 | 1099.62 | 6.15* | 6.15* | 6.15* |
| 0.5 | 1753.2 | 1684.14 | 1473.69 | 6.15* | 6.15* | 6.15* |
| 1 | 2803.95 | 2277.39 | 1887.84 | 6.15* | 6.15* | 6.15* |
| 2 | 3375 | 2930.55 | 2294.85 | 6.15* | 6.15* | 6.15* |
| 4 | 3516 | 3162 | 2984.04 | 6.15* | 6.15* | 6.15* |

Samples marked with an asterisks (*) indicate the value was below the LoD (which was 6.15 pg/ml).

TABLE 13
Secretion of IFNγ by CTX130 cells in the presence of CD70+
cell line ACHN.

| T cell: | IFNγ (pg/mL) | | | | | |
|---|---|---|---|---|---|---|
| ACHN ratio | CTX130 | | | Unedited T cells | | |
| 0 | 2.92 | 5.4 | 7.12 | 4.36 | 4.88 | 2.36* |
| 0.125 | 757.56 | 1369.96 | 981 | 2.92 | 7.12 | 8.36 |
| 0.25 | 1776.44 | 2668.04 | 2507.68 | 4.36 | 3.4 | 7.12 |
| 0.5 | 4508 | 6904 | 5248 | 8.36 | 7.12 | 7.12 |
| 1 | 11148 | 16568 | 13624 | 9.64 | 3.88 | 9.64 |
| 2 | 32460 | 52872 | 39228 | 5.96 | 7.12 | 8.36 |
| 4 | 67268 | 86620 | 64944 | 9.64 | 12.4 | 16.88 |

Samples marked with an asterisks (*) indicate the value was below the LoD (which was 2.36 pg/ml).

TABLE 14
Secretion of IL-2 by CTX130 cells in the presence of CD70+
cell line ACHN.

| T cell: | IL-2 (pg/mL) | | | | | |
|---|---|---|---|---|---|---|
| ACHN ratio | CTX130 | | | Unedited T cells | | |
| 0 | 4.48* | 4.48* | 4.48* | 4.48* | 4.48* | 4.48* |
| 0.125 | 247.16 | 367.2 | 266.4 | 4.48* | 4.48* | 4.48* |
| 0.25 | 455.16 | 651.6 | 552.92 | 4.48* | 4.48* | 4.48* |
| 0.5 | 961.76 | 1466.04 | 1326.48 | 4.48* | 4.48* | 4.48* |
| 1 | 2437.04 | 3337.08 | 2891.04 | 4.48* | 4.48* | 4.48* |
| 2 | 7180 | 12148 | 8388 | 4.48* | 4.48* | 4.48* |
| 4 | 12324 | 17040 | 13028 | 4.48* | 4.48* | 4.48* |

Samples marked with an asterisks (*) indicate the value was below the LoD (which was 4.48 pg/ml).

TABLE 15
No secretion of IFNγ by CTX130 cells in the presence of CD70-
cell line MCF7.

| T cell: | IFNγ (pg/mL) | | | | | |
|---|---|---|---|---|---|---|
| MCF7 ratio | CTX130 | | | Unedited T cells | | |
| 0 | 2.25* | 2.25* | 2.25* | 2.25* | 2.25* | 2.25* |
| 0.125 | 2.25* | 2.25* | 2.25* | 2.25* | 2.25* | 2.25* |
| 0.25 | 2.25* | 3.26 | 3.26 | 2.25* | 2.25* | 2.25* |
| 0.5 | 4.41 | 2.72 | 4.02 | 2.25* | 2.25* | 2.25* |
| 1 | 5.86 | 5.23 | 5.23 | 2.25* | 2.25* | 2.25* |
| 2 | 19.64 | 15.06 | 14.81 | 2.25* | 2.72 | 2.25 |
| 4 | 29.85 | 29.58 | 21.44 | 6.08 | 4.41 | 4.41 |

Samples marked with an asterisks (*) indicate the value was below the LoD (which was 2.25 pg/ml).

TABLE 16
No secretion of IL-2 by CTX130 cells in the presence of CD70−
cell line MCF7.

| T cell: | IL-2 (pg/mL) | | | | | |
|---|---|---|---|---|---|---|
| MCF7 ratio | CTX130 | | | Unedited T cells | | |
| 0 | 2.74* | 2.74* | 2.74* | 2.74* | 2.74* | 2.74* |
| 0.125 | 2.74* | 2.74* | 2.74* | 2.74* | 2.74* | 2.74* |
| 0.25 | 2.74* | 2.74* | 2.74* | 2.74* | 2.74* | 2.74* |
| 0.5 | 2.74* | 2.74* | 2.74* | 2.74* | 2.74* | 2.74* |
| 1 | 2.74* | 2.74* | 2.74* | 2.74* | 2.74* | 2.74* |
| 2 | 2.74* | 2.74* | 2.74* | 2.74* | 2.74* | 2.74* |
| 4 | 2.74* | 2.74* | 2.74* | 2.74* | 2.74* | 2.74* |

Samples marked with an asterisks (*) indicate the value was below the LoD (which was 2.74 pg/ml).

These results demonstrate that CTX130 cells exhibit effector function by secreting IFNγ and IL-2 in the presence of renal cell carcinoma cells expressing CD70, but not in the presence of the CD70 negative cell line MCF7.

### Example 7: Selective Killing of CD70+ Cells by Anti-CD70 CAR+ T Cells (CTX130)

The objective of this study was to assess the ability of CTX130 to selectively lyse CD70 expressing cells in vitro.

The ability of CTX130 or control T cells (unedited T cells with no anti-CD70 CAR expression) to specifically kill CD70 positive or CD70 negative target cells was assessed using a CellTiter-Glo luminescent cell viability-based cytotoxicity assay. A498 and ACHN cell lines were used as CD70 positive target lines, and the MCF7 cell line was used as a CD70 negative target line (all obtained from ATCC). T cells from the development lot 01 were used in these experiments.

50,000 human target cells (CD70 positive A498 and ACHN, CD70 negative MCF7) per well of an opaque-walled 96-well plate (Corning, Tewksbury, Mass.) were plated overnight. The next day, the cells were co-cultured with T cells at varying ratios (0.125:1, 0.25:1, 0.5:1, 1:1, 2:1 and 4:1 T cells to target cells) for 24 hours. Target cells were incubated with unedited T cells (TCR+ B2M+ CAR−), or CTX130 cells. After manually washing off T cells with PBS, the remaining viable target cells were quantified using a CellTiter-Glo luminescent cell viability assay (CellTiter-Glo® 2.0 Assay, Promega G9242). Fluorescence was measured using a Synergy H1 plate reader (Biotek Instruments, Winooski, Vt.). Prior to processing the cells for CellTiter-Glo analysis, supernatants were collected for quantification of cytokine secretion following co-culture.

The percent cell lysis was then calculated using the following equation using relative light units (RLU):

% Cell lysis=((RLU target cells with no effector−RLU target cells with effector))/(RLU target cell with no effector)×100

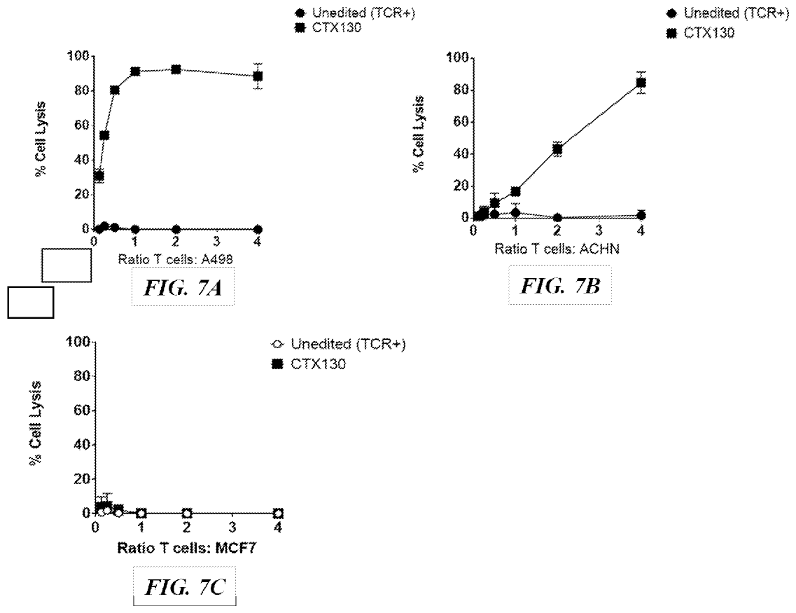

The development lot of CTX130 (lot 01) was tested for cell killing activity against the CD70+ cell lines A498 and ACHN. The CTX130 lot showed potent cell killing activity specifically against both high (A498; FIG. 7A) and low (ACHN; FIG. 7B) CD70 expressing cells, but not when co-cultured with CD70− MCF7 cells (FIG. 7C). In the absence of CAR expression, control unedited T cells were less effective at killing the CD70+ cells. See also data shown in Tables 17-19.

TABLE 17
Percent dead A498 cells in presence of CTX130 cells.

| T cell:A498 cell ratio | CTX130 | | | Unedited T cells | | |
|---|---|---|---|---|---|---|
| 0.125 | 33.6 | 32.8 | 26.5 | −3.1 | −0.8 | 0.3 |
| 0.25 | 55.6 | 53.1 | 54.3 | −1.2 | 2.7 | 3.1 |
| 0.5 | 82.4 | 80.7 | 78.5 | −3.5 | 1.8 | 1.4 |
| 1 | 92.0 | 90.3 | 91.4 | −6.5 | −1.5 | −2.6 |
| 2 | 94.5 | 91.3 | 91.6 | −6.0 | −1.1 | −1.0 |
| 4 | 87.7 | 81.8 | 96.0 | −7.4 | −5.9 | −6.7 |

TABLE 18
Percent dead ACHN cells in presence of CTX130 cells.

| T cell:ACHN cell ratio | CTX130 | | | Unedited T cells | | |
|---|---|---|---|---|---|---|
| 0.125 | 3.8 | −1.3 | −0.9 | 2.7 | −2.9 | 3.1 |
| 0.25 | 7.5 | 0.2 | 4.2 | 4.6 | −1.6 | 1.3 |
| 0.5 | 15.9 | 3.4 | 9.2 | 4.1 | 3.5 | −0.9 |
| 1 | 18.1 | 14.5 | 17.5 | 0.3 | 10.3 | −0.9 |
| 2 | 43.1 | 38.9 | 47.8 | −0.8 | −0.4 | 1.4 |
| 4 | 86.3 | 77.3 | 90.5 | −5.6 | 5.6 | −3.7 |

TABLE 19
Percent dead MCF7 cells in presence of CTX130 cells.

| T cell:MCF7 cell ratio | CTX130 | | | Unedited T cells | | |
|---|---|---|---|---|---|---|
| 0.125 | 10.8 | −4.4 | 0.2 | −0.7 | 1.9 | −1.0 |
| 0.25 | 13.0 | −10.2 | −0.3 | 2.6 | 2.8 | −0.1 |
| 0.5 | 5.6 | −12.3 | −7.1 | 0.8 | −1.4 | −9.5 |
| 1 | 0.6 | −15.3 | −10.3 | −1.0 | −3.7 | −12.5 |
| 2 | 0.7 | −22.6 | −10.6 | −3.5 | −8.1 | −13.7 |
| 4 | 0.1 | −26.2 | −16.2 | −12.8 | −10 | −20.5 |

These results demonstrated that CTX130 cells were able to lyse cancer cell lines in vitro in a CD70− specific manner.

### Example 8: Efficacy of Anti-CD70 CART Cells: The Subcutaneous Renal Cell Carcinoma Tumor Xenograft Model in NOG Mice

The ability of T cells expressing a CD70 CAR to eliminate kidney carcinoma cells that express high levels of CD70 was evaluated in in vivo using subcutaneous renal cell carcinoma tumor xenograft models in mice. These models included a subcutaneous A498-NOG model, a subcutaneous 786-O-NSG model, a subcutaneous Caki-2-NSG model, and a subcutaneous Caki-1-NSG model. CTX130 cells were produced as described herein.

For each subcutaneous renal cell carcinoma tumor xenograft model, five million cells of the indicated cell type were injected subcutaneously into the right flank of NOG (NOD.Cg-Prkdc^{scid}Il2rg^{tm1Sug}/JicTac) mice. When mean tumor size reached an average size of approximately 150 mm³, mice were either left untreated or injected intravenously with 8×10⁶ CAR⁺ CTX130 (TRAC⁻/B2M⁻/CD70⁻/anti-CD70 CAR+ T cells) cells per mouse. In the subcutaneous A498-NOG model, an additional group of mice was injected with 7.5×10⁶ CAR+ TRAC⁻B2M⁻ anti-CD70 CAR-T cells per mouse.

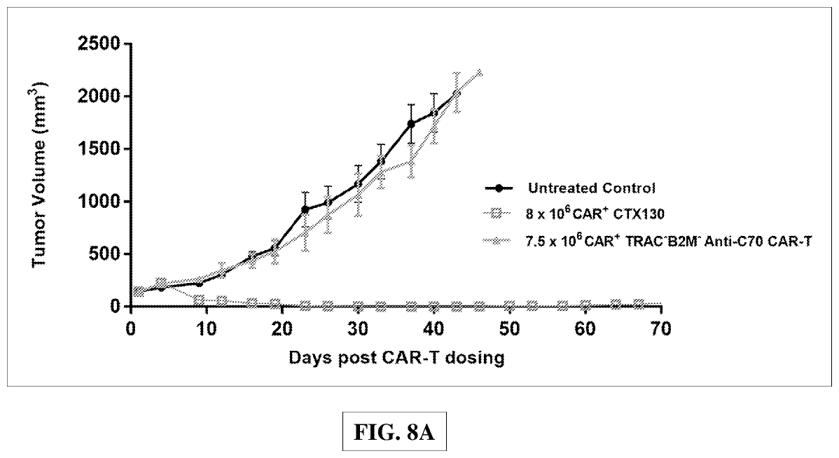

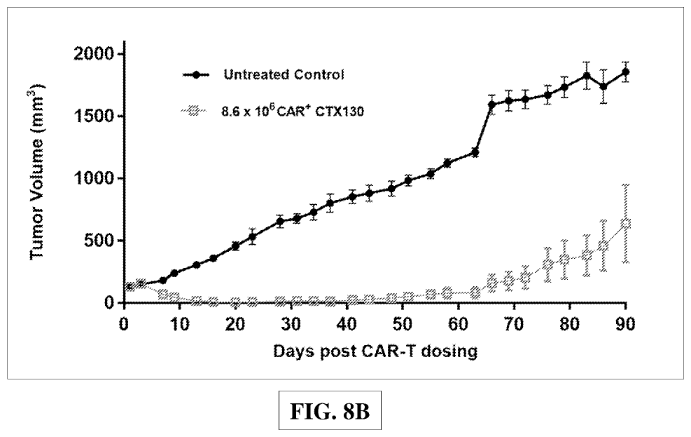

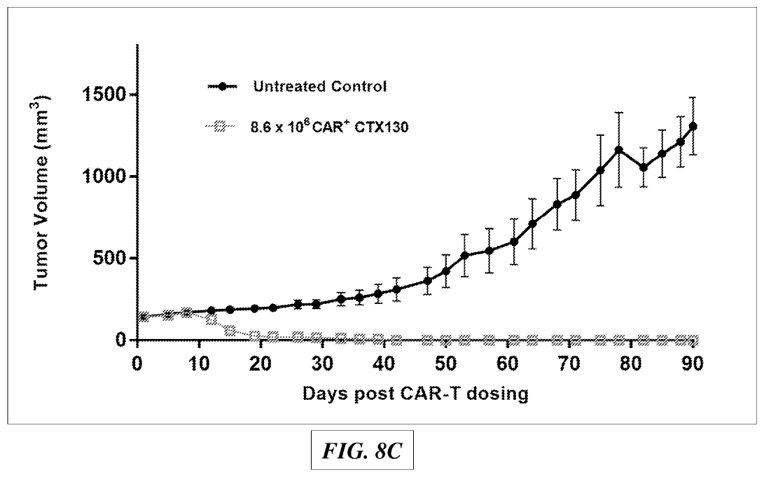

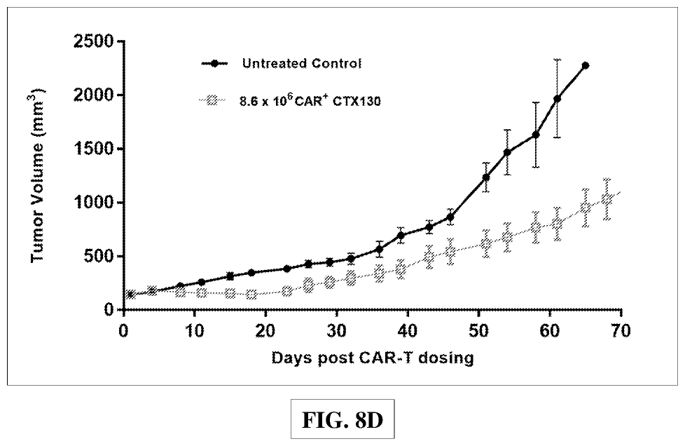

The CTX130 cells completely eliminated tumor growth in the subcutaneous A498-NOG model (FIG. 8A) and the subcutaneous Caki-2-NSG model (FIG. 8C). Tumor growth in mice injected with TRAC⁻/B2M⁻/anti-CD70 CAR+ T cells was similar to that of the untreated control mice (FIG. 8A). CTX130 cells significantly reduced tumor growth in the subcutaneous 786-O-NSG model (FIG. 8B) and the subcutaneous Caki-1-NSG model (FIG. 8D).

Taken together, these results demonstrate that CTX130 cells reduced tumor growth in four types of subcutaneous renal cell carcinoma tumor xenograft models.

Tumor Re-Challenge Model Renal Cell Carcinoma Tumor Xenograft Model

The efficacy of CTX130 was also tested in a subcutaneous A498 xenograft model with re-challenge. In brief, five million A498 cells were injected subcutaneously in the right flank of NOD (NOD.Cg-Prkdc^{scid}Il2rg^{tm1Sug}/JicTac) mice. Tumors were allowed to grow to an average size of approximately 51 mm³ after which the tumor-bearing mice were randomized in two groups (N=5/group). Group 1 was left untreated while Group 2 received 7×10⁶ CAR+ CTX130 cells and Group 3 received 8×10⁶ CAR+ TRAC− B2M− anti-CD70 CAR T cells. On Day 25, a tumor re-challenge was initiated whereby 5×10⁶ A498 cells were injected into the left flank of treated mice and into a new control group (Group 4).

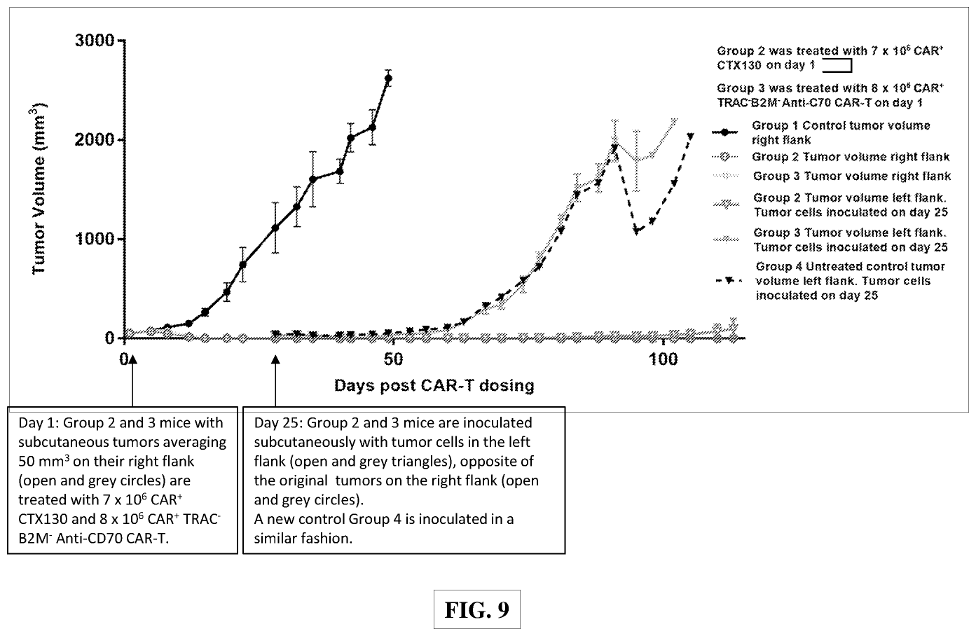

As shown in FIG. 9, mice treated with CTX130 cells exhibited no tumor growth post rechallenge by injection of A498 cells into the left flank while mice treated with anti-CD70 CAR T cells exhibited tumor growth of the A498 cells injected into the left flank. These results demonstrate that CTX130 cells retain higher in vivo efficacy after re-exposure to tumor cells than other anti-CD70 CAR+ T cells (CAR+ TRAC− B2M− anti-CD70 CAR T cells).

Efficacy of CTX130 Redosing Renal Cell Carcinoma Tumor Xenograft Model

The efficacy of CTX130 was also tested in a subcutaneous A498 xenograft model with redosing. In brief, five million A498 cells were injected subcutaneously into the right flank of NOG (NOD.Cg-Prkdc^{scid}Il2rg^{tm1Sug}/JicTac) mice. When mean tumor size reached an average size of approximately 453 mm³, mice were either left untreated or injected intravenously (N=5) with 8.6×10⁶ CAR+ CTX130 cells per mouse. Group 2 mice were treated with a second and third dose of 8.6×10⁶ CAR+ CTX130 cells per mouse on day 17 and 36, respectively. Group 3 mice were treated with a second dose of 8.6×10⁶ CAR+ CTX130 cells per mouse on day 36.

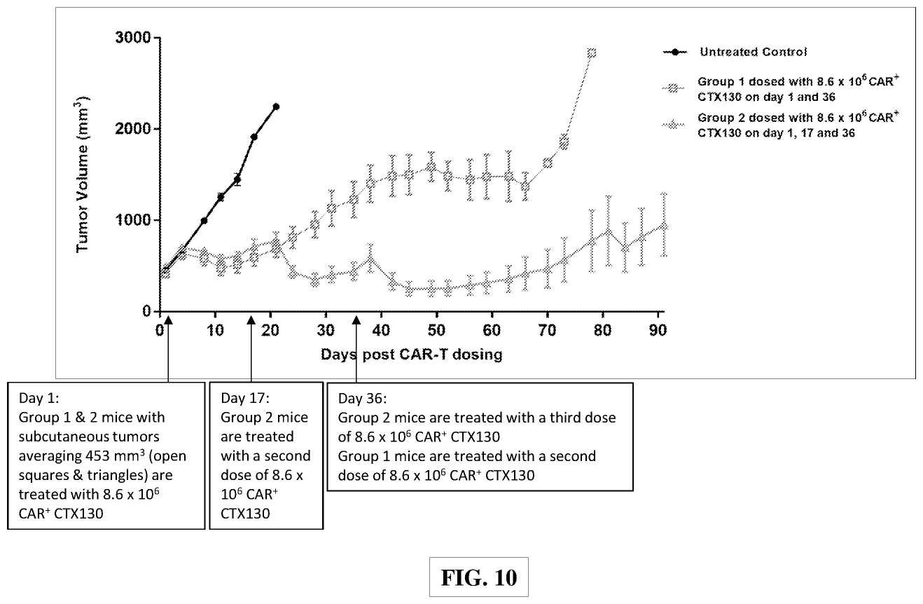

As shown in FIG. 10, mice dosed with CTX 130 cells on day 1 and then redosed on day 17 and 36 exhibited less tumor growth than mice administered only one redose on day 36. These results demonstrate that redosing of CTX130 cells provides enhanced suppression of tumor growth.

### Example 9: A Phase 1, Open-Label, Multicenter, Dose Escalation and Cohort Expansion Study of the Safety and Efficacy of Allogeneic CRISPR-Cas9 Engineered T Cells (CTX130) in Adult Subjects with Advanced, Relapsed or Refractory Renal Cell Carcinoma (RCC) with Clear Cell Differentiation

CTX130 is a CD70− directed T-cell immunotherapy comprised of allogeneic T cells that are genetically modified ex vivo using CRISPR-Cas9 (clustered regularly interspaced short palindromic repeats/CRISPR-associated protein 9) gene editing components (single guide RNAs [sgRNAs] and Cas9 nuclease). The modifications include targeted disruption of the T-cell receptor alpha constant (TRAC), beta 2-microglobulin (B2M), and CD70 loci and the insertion of an anti-CD70 chimeric antigen receptor (CAR) transgene into the TRAC locus via an adeno-associated virus (AAV) expression cassette. The anti-CD70 CAR (SEQ ID NO: 46) is composed of an anti-CD70 single-chain variable fragment derived from a previously characterized anti-CD70 hybridoma IF6 (SEQ ID NO: 48), the CD8 transmembrane domain (SEQ ID NO: 54), a 4-1BB co-stimulatory domain (SEQ ID NO: 57), and a CD3ζ signaling domain (SEQ ID NO: 61).

### 1. Study Overview

### 1.1 Study Population

Dose escalation and cohort expansion include adult subjects with advanced (e.g., unresectable or metastatic), relapsed or refractory renal cell carcinoma (RCC) with clear cell differentiation (e.g., predominantly). These include subjects who have had prior exposure to both a checkpoint inhibitor (CPI) and a vascular endothelial growth factor (VEGF) inhibitor.

### 1.2 Mode of Administration

Subjects received an intravenous (IV) infusion of CTX130 following lymphodepleting (LD) chemotherapy.

### 1.3 Duration of Subject Participation

Subjects participate in this study for approximately 5 years. After completion of this study, all subjects are required to participate in a separate long-term follow-up study for an additional 10 years to assess safety and survival.

### 2. Study Purpose

The purpose of the Phase 1 dose escalation study is to evaluate the safety and efficacy of anti-CD70 allogeneic CRISPR-Cas9 engineered T cells (CTX130) in subjects with advanced (unresectable or metastatic), relapsed or refractory RCC with clear cell differentiation.

CAR T-cell therapies are adoptive T-cell therapeutics (ACTs) used to treat human malignancies. Although CAR T-cell therapy has led to tremendous clinical success, including durable remission in patients with relapsed/refractory non-Hodgkin lymphoma (NHL) and pediatric patients with acute lymphoblastic leukemia (ALL), their investigational use in solid tumor indications has not yet shown relevant clinical response. In addition, currently approved ACTs are autologous and require patient-specific cell collection and manufacturing, which has led to reintroduction of residual contaminating tumor cells from engineered T cells (Ruella et al., 2018). Also, low response rates in patients with chronic lymphocytic leukemia (CLL) and lack of responses in patients with B-cell ALL treated with autologous CAR T cell therapy have been partially attributed to the exhausted T cell phenotype (Fraietta et al., (2018) Nat Med 24, 563-571; Riches et al., (2013) Blood, 121, 1612-21; Mackall C. L., (2019) Cancer Research, AACR annual meeting, Abstract PL01-05; Long et al., (2015) Nat Med, 21, 581-90; Walker et al., (2017) Mol Ther, 25, 2189-2201; Zheng et al., (2018) Drug Discov Today, 23, 1175-1182.

Finally, collection, shipment, manufacturing, and shipment back to the patient's treating physician is time-consuming and, as a result, some patients have experienced disease progression or death while awaiting treatment. An allogeneic off-the-shelf CAR T cell product could provide benefits such as immediate availability, lack of manufacturing failures, and chemotherapy-naïve T cells from healthy donors, thus a more consistent product relative to autologous CAR T cell therapies.

With CRISPR-Cas9 editing, disruption of the endogenous T cell receptor (TCR) and major histocompatibility complex (MHC) class I proteins can be achieved. TCR knockout is intended to significantly reduce or eliminate the risk of graft versus host disease (GvHD), whereas MHC knockout is designed to increase CAR T cell persistence. This first-in-human trial in subjects with unresectable or metastatic ccRCC evaluates the safety and efficacy of this CRISPR-Cas9-modified allogeneic CAR T cell approach.

CTX130, a CD70− directed genetically modified allogeneic T-cell immunotherapy, is manufactured from the cells of healthy donors; therefore, the resultant manufactured cells are intended to provide each subject with a consistent, final product of reliable quality. Furthermore, the manufacturing of CTX130, through precise delivery and insertion of the CAR at the TRAC site using AAV and homology-directed repair (HDR), does not present the risks associated with random insertion of lentiviral and retroviral vectors.

Finally, CD70 is the membrane-bound ligand of the CD27 receptor, which belongs to the tumor necrosis factor receptor (TNFR) superfamily. It is commonly expressed at elevated levels in multiple carcinomas and lymphomas, and it is a diagnostic biomarker for ccRCC.

### 3. Study Objectives

Primary objective, Part A (Dose escalation): To assess the safety of escalating doses of CTX130 in subjects with unresectable or metastatic ccRCC to determine the recommended Part B dose (RPBD).

Primary objective, Part B (Cohort expansion): To assess the efficacy of CTX130 in subjects with unresectable or metastatic ccRCC as measured by objective response rate (ORR) according to the Response Evaluation Criteria in solid tumors (RECIST 1.1).

Secondary objectives (Parts A and B): To further characterize the efficacy of CTX130 over time; to further assess the safety of CTX130 and describe and assess adverse events (AEs) of special interest (AESIs), including cytokine release syndrome (CRS), tumor lysis syndrome (TLS) and GvHD; and to characterize pharmacokinetics (PK) (expansion and persistence) of CTX130 in blood.

Exploratory objectives (Parts A and B): To identify genomic, metabolic, and/or proteomic biomarkers that are associated with disease, clinical response, resistance, safety, or pharmacodynamic (PD) activity; to further describe the kinetics of efficacy of CTX130; and to describe the effect of CTX130 on patient-reported outcome (PRO).

### 4. Study Eligibility

### 4.1 Inclusion Criteria

To be considered eligible to participate in this study, a subject must meet all the inclusion criteria listed below:
1. ≥18 years of age and body weight ≥60 kg.
2. Able to understand and comply with protocol-required study procedures and voluntarily sign a written informed consent document.
3. Diagnosed with unresectable or metastatic clear cell RCC with clear cell differentiation:
   Have previous exposure to both a CPI and a VEGF inhibitor and documented progression after adequate exposure for favorable risk by International Metastatic RCC Database Consortium (IMDC) criteria or a lack of response after adequate exposure for intermediate and poor risk characteristics.
   Have local confirmation of clear cell RCC on biopsy (within 3 months of enrollment or during screening).
   Availability of tumor tissues.
   Have measurable disease as assessed by the site radiologist per RECISTv1.1. Target lesions situated in a previously irradiated area are considered measurable if progression has been demonstrated in such lesions.
   Have at least one nontarget lesion that is suitable for biopsies.
4. Karnofsky Performance Status (KPS) ≥80% as assessed during the screening period.
5. Meets protocol-specified criteria to undergo LD chemotherapy and CAR T cell infusion.
6. Adequate organ function:
   Renal: Creatinine clearance (CrCl) ≥50 mL/min
   Liver:
      Aspartate aminotransferase (AST) and alanine aminotransferase (ALT) <3× upper limit of normal (ULN);
      Total Bilirubin <2×ULN (for Gilbert's syndrome, total bilirubin <3 mg/dL); and normal conjugated bilirubin,
      Albumin ≥90% of lower limit of normal.
   Cardiac: Hemodynamically stable and left ventricular ejection fraction (LVEF) ≥45% by echocardiogram.
   Pulmonary: Oxygen saturation level on room air >90% per pulse oximetry.
   Hematologic: Platelet count >100,000/mm³, absolute neutrophil count >1500/mm³, and hemoglobin (HgB) >9 g/dL without prior blood cell transfusion before screening
   Coagulation: Activated Partial Thromboplastin Time (aPTT) or PTT ≤1.5×ULN
7. Female patients of childbearing potential (postmenarcheal, has an intact uterus and at least 1 ovary, and is less than 1 year postmenopausal) must agree to use a highly effective method of contraception (as specified in the protocol) from enrollment through at least 12 months after the last CTX130 infusion.
8. Male patients must agree to use an effective method of contraception (as specified in the protocol) from enrollment through at least 12 months after the last CTX130 infusion.

### 4.2 Exclusion Criteria

To be eligible for entry into the study, the subject must not meet any of the exclusion criteria listed below:

1. Prior treatment with any anti-CD70 targeting agents.

2. Prior treatment with any CAR T cells or any other modified T or natural killer (NK) cells.

3. Known contraindications to any LD chemotherapy agent(s) or any of the excipients of CTX130 product.

4. Subjects with central nervous system (CNS) manifestation of their malignancy as evidenced by positive screening MRI or past history.

5. History or presence of clinically relevant CNS pathology such as seizure, stroke, severe brain injury, cerebellar disease, history of posterior reversible encephalopathy syndrome (PRES) with prior therapy, or another condition that may increase CAR T-cell related toxicities.

6. Ongoing, clinically significant pleural effusion or ascites or any pericardial infusion or a history of pleural effusion or ascites in the last 2 months.

7. Unstable angina, clinically significant arrhythmia, or myocardial infarction within 6 months prior to screening.

8. Diabetes mellitus with currently hemoglobin A1c (HbA1c) level of 7.0% or 48 mmol/mL.

9. Uncontrolled, acute life-threatening bacterial, viral, or fungal infection.

10. Positive for presence of human immunodeficiency virus type 1 or 2, or active hepatitis B virus or hepatitis C virus infection. Subjects with prior history of hepatitis B or C infection who have documented undetectable viral load (by quantitative polymerase chain reaction or nucleic acid testing) are permitted.

11. Previous or concurrent malignancy, except those treated with curative approach not requiring systemic therapy and has been in remission for >12 months, or any other localized malignancy that has a low risk of developing into metastatic disease.

12. Primary immunodeficiency disorder or active autoimmune disease requiring steroids and/or any other immunosuppressive therapy.

13. Prior solid organ transplantation or bone marrow transplant.

14. Use of anti-tumor or investigational agent, including radiotherapy, within 14 days prior to enrollment. Use of physiological doses of steroids are permitted for subjects previously on steroids if clinically indicated and in consultation with the medical monitor.

15. Received live vaccines or herbal medicines as part of traditional Chinese medicine or non-over-the-counter herbal remedies within 28 days prior to enrollment.

16. Diagnosis of significant psychiatric disorder that could seriously impede the subject's ability to participate in the study.

17. Pregnant or breastfeeding females.

### 5. Study Design

### 5.1 Investigational Plan

This is a single-arm, open-label, multicenter, Phase 1 study evaluating the safety and efficacy of CTX130 in subjects with metastatic RCC. The study is divided into 2 parts: dose escalation (Part A) followed by cohort expansion (Part B).

In Part A, dose escalation begins in adult subjects diagnosed with unresectable or metastatic ccRCC with clear cell differentiation who have had progressed to both a CPI and a vascular endothelial growth factor (VEGF) inhibitor. Dose escalation is performed according to the criteria described herein.

In Part B, an expansion cohort is initiated to further assess the safety and efficacy of CTX130 using an optimal Simon 2-stage design. In the first stage, at least 23 subjects are treated with the recommended dose of CTX130 for Part B cohort expansion (at or below the MTD determined in Part A).

5.1.1 Study Design

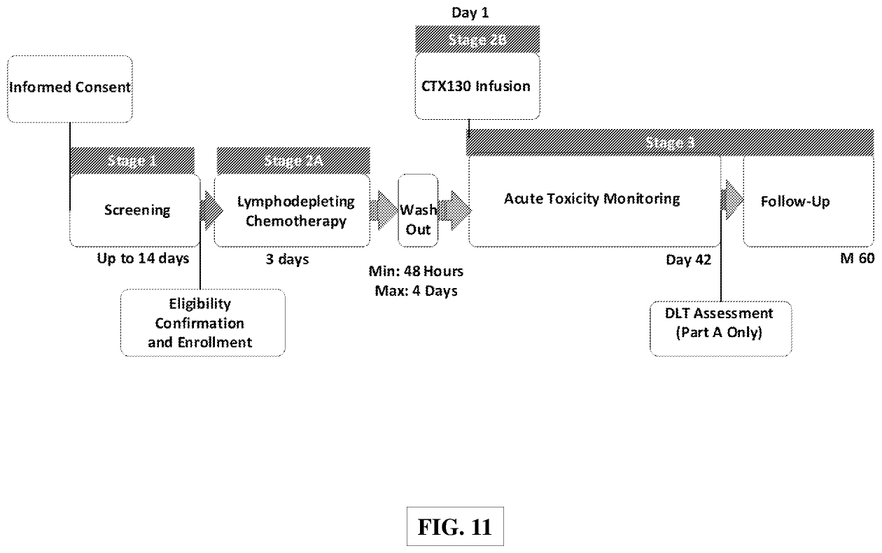

The study is divided into 2 parts: dose escalation (Part A) followed by cohort expansion (Part B). Both parts of the study consist of 3 main stages: screening, treatment, and follow-up. A schematic depiction of the study schema is shown in FIG. 11.

The 3 main stages are as follows:

Stage 1—Screening to determine eligibility for treatment (up to 14 days).
Stage 2—LD chemotherapy and infusion of CTX130.
Stage 2A—LD chemotherapy: Co-administration of fludarabine 30 mg/m² and cyclophosphamide 500 mg/m² intravenously (IV) daily for 3 days. Both agents are started on the same day and administered for 3 consecutive days. LD chemotherapy must be completed at least 48 hours (but no more than 7 days) prior to CTX130 infusion.
Stage 2B—CTX130 infusion
Clinical eligibility—Prior to both the initiation of LD chemotherapy and infusion of CTX130, subjects' clinical eligibility must be reconfirmed.
   Stage 3—Follow up (5 years after the last CTX130 infusion).

During the post-CTX130 infusion period, subjects are monitored for acute toxicities (Days 1-28), including CRS, immune effector cell-associated neurotoxicity syndrome (ICANS), GvHD, and other AEs. Toxicity management guidelines are described herein (see Section 8). During Part A (dose escalation), subjects are hospitalized for the first 7 days following CTX130 infusion, or longer if required by local regulation or site practice. In both Part A and Part B, subjects must remain within proximity of the investigative site (i.e., 1-hour transit time) for 28 days after CTX130 infusion.

After the acute toxicity observation period, subjects are subsequently followed for up to 5 years after the last CTX130 infusion with physical exams, regular laboratory and imaging assessments, and AE assessments. After completion of this study, subjects are required to participate in a separate long-term follow-up study for an additional 10 years to assess long-term safety and survival.

5.1.2 Study Subjects

Up to 24 subjects are to be treated in Part A (dose escalation).

Approximately 71 subjects are to be treated in Part B (cohort expansion), contingent upon the outcome of an interim analysis.

5.1.3 Study Duration

Subjects participate in this study for up to 5 years. After completion of this study, subjects are required to participate in a separate long-term follow-up study for an additional 10 years to assess long-term safety and survival.

### 5.2 CTX130 Dose Escalation

The following doses of CTX130, based on the number of CAR⁺ T cells, may be evaluated in this study (Table 20), starting with Dose Level 1 (DL1). A dose limit of 1×10⁵ TCR⁺ cells/kg may be imposed for all dose levels.

TABLE 20
Dose Escalation of CTX130.

| | Total CAR⁺ |
|---|---|
| Dose Level | T-Cell Dose |
| −1 | 1 × 10⁶ |
| (de-escalation) | |
| 1 | 3 × 10⁷ |
| 2 | 1 × 10⁸ |
| 3 | 3 × 10⁸ |
| 4 | 9 × 10⁸ |

CAR: chimeric antigen receptor.

Dose escalation is performed using a standard 3+3 design in which 3 to 6 subjects are enrolled at each dose level depending on the occurrence of dose-limiting toxicities (DLTs) after the initial dosing, as defined herein. The DLT evaluation period begins with initial CTX130 infusion and last for 28 days. In Dose Level 1 (and Dose Level −1, if required), subjects are to be treated in a staggered manner, such that a subject will only receive CTX130 once the previous subject has completed the DLT evaluation period (e.g., staggered by 28 days). In the event of a DLT at Dose Level 1 requiring decreased dosing to Dose Level −1, dosing of all subjects at Dose Level −1 will also be staggered by 28 days. If no DLT occurs at Dose Level 1, dose escalation will progress to Dose Level 2, and dosing between each subject will be staggered by 14 days. If no DLT occurs at the first 2 dose levels (Dose Levels 1 and 2), at subsequent dose levels (Dose Levels 3 and 4) dosing will be staggered by 7 days between each subject.

Dose escalation is performed according to the following rules:
If 0 of 3 subjects experience a DLT, escalate to the next dose level.
If 1 of 3 subjects experiences a DLT, expand the current dose level to 6 subjects.
   If 1 of 6 subjects experiences a DLT, escalate to the next dose level.
   If ≥2 of 6 subjects experience a DLT:
      If in Dose Level −1, evaluate alternative dosing schema or declare inability to determine recommended dose for Part B cohort expansion.
      If in Dose Level 1, de-escalate to Dose Level −1.
      If in Dose Level 2-4, declare previous dose level the MTD.
If ≥2 of 3 subjects experience a DLT:
   If in Dose Level −1, evaluate alternative dosing schema or declare inability to determine the recommended dose for Part B cohort expansion.
   If in Dose Level 1, decrease to Dose Level −1.
   If in Dose Level 2-4, declare previous dose level the MTD.
Intermediate doses between DL2 and DL3, e.g., 1.5×10⁸ CAR⁺ T cells may be allowed.
Intermediate doses between DL3 and DL4, e.g., 4.5×10⁸ CAR⁺ T cells, 6×10⁸ CAR⁺ T cells, or 7.5×10⁸ CAR⁺ T cells, may be allowed, which may be based on review of DL4 safety and efficacy data.
No dose escalation may be beyond highest dose listed in Table 20 in this study.

5.2.1 Maximum Tolerated Dose Definition

The MTD is the highest dose for which DLTs are observed in fewer than 33% of subjects. An MTD may not be determined in this study. A decision to move to the Part B expansion cohort may be made in the absence of an MTD provided the dose is at or below the maximum dose studied (or MAD) in Part A of the study.

5.2.2 DLT Definitions

Toxicities are graded and documented according to National Cancer Institute (NCI) Common Terminology Criteria for Adverse Events (CTCAE) version 5.0, except for CRS (ASTCT criteria; American Society for Transplantation and Cellular Therapy criteria; Lee criteria), neurotoxicity (ICANS criteria; immune effector cell-associated neurotoxicity syndrome criteria, CTCAE version 5.0; Lee criteria), and GvHD (MAGIC criteria; Mount Sinai Acute GvHD International Consortium criteria; Harris et al., (2016) Biol Blood Marrow Transplant 22, 4-10). AEs that have no plausible causal relationship with CTX130 are not considered DLTs.

A DLT is defined as:
A. Grade ≥2 GvHD if it does not respond to steroid treatment (e.g., 1 mg/kg/day) within 7 days (GvHD grading is provided in Table 31).
B. Any CTX130-related Grade 3 to 5 toxicity occurring within 28 days immediately after infusion of CTX130, with the exceptions tabulated below:

The following are NOT be considered as DLTs:
Any Grade 3 or 4 CRS according to the CRS Grading System that improves to Grade ≤2 with appropriate medical intervention within 72 hours
Grade 3 or 4 fever resolving within 72 hours with appropriate medical intervention
Grade 3 fatigue lasting <7 days
Any Grade 3 or 4 abnormal liver function tests that improve to Grade ≤2 within 14 days
   Any Grade 3 toxicity involving vital organs other than cardiac (e.g., pulmonary, renal) that improves to Grade ≤2 within 7 days
   Any Grade 3 cardiac toxicity that improves to Grade ≤2 within 72 hours
   Any Grade 3 neurotoxicity that revolves within 72 hours to Grade ≤2
   Death due to disease progression
   GvHD that is not steroid-refractory and revolves to Grade 1 within 14 days
      5.3 Repeat Dosing with CTX130 in Part A and Part B

This study will allow for no more than 2 times redosing of subjects with CTX130 cells. To be considered for redosing, subjects must have either 1) achieved a partial response (PR) or complete response (CR) after initial or second CTX130 infusion and subsequently progressed within 2 years of last dose, even without meeting the formal RECIST criteria for progression, or 2) achieved PR (but not CR) or stable disease (SD) at the Month 3 study visit after the most recent CTX130 infusion (redosing decisions will be based upon local CT scan/assessment).

The earliest time at which a subject could be redosed is 2 months after the initial or second CTX130 infusion.

To be redosed with CTX130, subjects shall meet the following criteria:
Confirmation tumor is CD70⁺ at relapse (based on local or central assessment) if a lesion is available that is amenable to biopsy
No prior DLT during dose escalation (if applicable)
No prior Grade ≥3 CRS without resolution to Grade ≤2 within 72 hours following CTX130 infusion
No prior Grade >1 GvHD following CTX130 infusion
No prior Grade ≥2 ICANS following CTX130 infusion
Meet initial study inclusion criteria (#1, #2, #4-8) and exclusion criteria (#2 [except prior treatment with CAR T cells]-17) as described in herein (see Section 4).
Meet criteria for LD chemotherapy and CTX130 infusion as described in this Example.

Subjects who are redosed should be followed consistent with the initial dosing. All screening assessments must be repeated, including brain MRI.

Additional redosing considerations include the following:
The CT scan demonstrating disease relapse/progression will serve as the new baseline for tumor response evaluation. Redosing must occur within 28 days of that scan.
If a subject remains in PR at Month 3 visit and is redosed, the original baseline scan will continue to be used for tumor response evaluation.
Subjects in the dose escalation cohorts who undergo redosing will receive the highest CTX130 dose that has been deemed safe.
Subjects in the expansion cohort will be redosed with the recommended Part B dose.

Prior to each dosing event, subjects may receive another dose of LD chemotherapy.

### 6. Study Procedures

Both the dose escalation and expansion parts of the study consists of 3 distinct stages: (1) screening and eligibility confirmation, (2) LD chemotherapy and CTX130 infusion, and (3) follow-up. During the screening period, subjects are assessed according to the eligibility criteria described herein. After enrollment, subjects receive LD chemotherapy, followed by infusion of CTX130. After completing the treatment period, subjects are assessed for RCC response, disease progression, and survival. Throughout all study periods, subjects are regularly monitored for safety.

A complete schedule of assessments is provided in Table 21 and Table 22. Descriptions of all required study procedures are provided herein. In addition to protocol-mandated assessments, subjects should be followed per institutional guidelines, and unscheduled assessments should be performed when clinically indicated. Missed evaluations should be rescheduled and performed as close to the originally scheduled date as possible. An exception is made when rescheduling becomes, in the healthcare practitioner's opinion, medically unnecessary or unsafe because it is too close in time to the next scheduled evaluation. In that case, the missed evaluation should be abandoned.

For the purposes of this protocol, there is no Day 0. All visit dates and windows are to be calculated using Day 1 as the date of CTX130 infusion.

TABLE 21
Schedule of Assessments (Screening to Month 24) for both Part A and Part B of Study.

| | Assessment | | | | | | | | | |
|---|---|---|---|---|---|---|---|---|---|---|
| | | | | Follow-Up | | | | | | |
| | | LD | CTX130 | | | | | | | |
| | Screening ¹ | Chemo² | Infusion 3 | | | | | | | |
| | Day | | | | | | | | | |
| | | | | | | | Day7 ± | Day10 ± | Day15 ± | Day22 ± |
| | | | Day1 | Day2 | Day3 | Day5 | 2 d | 2 d | 2 d | 2 d |
| Eligibility | X | X | X | | | | | | | |
| Confirmation⁴ | | | | | | | | | | |
| Informed | X | | | | | | | | | |
| consent | | | | | | | | | | |
| Medical history ⁵ | X | | | | | | | | | |
| Physical exam⁶ | X | X | X | X | X | X | X | X | X | X |
| Vital signs ⁷ | X | X | X | X | X | X | X | X | X | X |
| Height, weight ⁸ | X | X | X | | | | | | | |
| Pregnancy test ⁹ | X | X | | | | | | | | |
| Brain MRI¹⁰ | X | | | | | | | | | |
| Karnofsky | X | | X | | X | | X | | X | X |
| Performance | | | | | | | | | | |
| Status (KPS) | | | | | | | | | | |
| Echocardiogram | X | | | | | | | | | |
| 12-leadECG ¹¹ | X | X | X | | | | | | | |
| ICE assessment ¹² | X | | X | X | X | X | X | X | X | X |
| PRO¹³ | X | | X | | | | X | | X | X |
| Concomitant | X | X | X | X | X | X | X | X | X | X |
| medications ¹⁴ | | | | | | | | | | |
| AEs ¹⁵ | X | X | X | X | X | X | X | X | X | X |
| Hospital | | | | | Continuous | | | | | |
| utilization | | | | | | | | | | |

Metastatic ccRCC Disease/Response Assessments (Central)

| | | |
|---|---|---|
| CT scan ¹⁶ | X | |
| Tumor biopsy ^{17, 18} | X | X |

Laboratory Assessments (Local)

| | | | | | | | | | | |
|---|---|---|---|---|---|---|---|---|---|---|
| CBC w/ | X | X | X | X | X | X | X | X | X | X |
| differential | | | | | | | | | | |
| Serum | X | X | X | X ¹⁹ | X ¹⁹ | X ¹⁹ | X ¹⁹ | X ¹⁹ | X ¹⁹ | X ¹⁹ |
| chemistry ¹⁹ | | | | | | | | | | |
| Coagulation | X | X | X | X | X | X | X | X | X | X |
| parameters | | | | | | | | | | |
| Viral serology²⁰ | X | | | | | | | | | |
| Lymphocyte | X | X | X | | | X | | | X | X |
| subsets²¹ | | | | | | | | | | |
| Ferritin, CRP, | X | | X | X | X | X | X | X | X | X |
| Triglyceride | | | | | | | | | | |

Biomarkers (Blood, Central)

| CTX130 levels ²² | | X | | X²³ | X | X | X | X | X | X |
|---|---|---|---|---|---|---|---|---|---|---|
| | | | | pre/post | | | | | | |
| Cytokines ²⁴ | X | | X | X | X | X | X | X | X | X |
| BSAP, PINP²⁵ | X | | X | | | | X | | X | X |
| Anti-CTX130 | X | | | | | | | | | |
| Ab | | | | | | | | | | |
| Cell-free DNA | X | | | | | | | | | |
| Exploratory | X²⁷ | X²⁸ | X | X | X | X | X | X | X | X |
| biomarkers ²⁶ | | | | | | | | | | |
| | Assessment | | | | | | | | | |
| | Follow-Up | | | | | | | | | |
| | Day | | | | | | | | | |
| | | | M2/ | M3/ | M6/ | M9/ | M12/ | M15/ | M18/ | M24/ |
| | | | Day | Day | Day | Day | Day | Day | Day | Day |
| | M1/ | Day42 ± | 56 ± | 84 ± | 168 ± | 252 ± | 336 ± | 420 ± | 504 ± | 672 ± |
| | Day28 ± 2 d | 2 d | 7 d | 7 d | 14 d | 14 d | 14 d | 14 d | 14 d | 21 d |
| Eligibility | | | | | | | | | | |
| Confirmation⁴ | | | | | | | | | | |
| Informed | | | | | | | | | | |
| consent | | | | | | | | | | |
| Medical history ⁵ | | | | | | | | | | |
| Physical exam⁶ | X | X | X | X | X | X | X | X | X | X |
| Vital signs ⁷ | X | X | X | X | X | X | X | X | X | X |
| Height, weight ⁸ | X | | | | | | | | | |
| Pregnancy test ⁹ | X | | X | X | | | | | | |
| Brain MRI¹⁰ | | | | | | | | | | |
| Karnofsky | X | X | | X | X | X | X | X | X | X |
| Performance | | | | | | | | | | |
| Status (KPS) | | | | | | | | | | |
| Echocardiogram | | | | | | | | | | |
| 12-leadECG ¹¹ | | X | | | | | | | | |
| ICE assessment ¹² | X | X | X | | | | | | | |
| PRO¹³ | X | X | X | X | X | X | X | X | X | X |
| Concomitant | X | X | X | X | X | X | X | X | X | X |
| medications ¹⁴ | | | | | | | | | | |
| AEs ¹⁵ | X | X | X | X | X | X | X | X | X | X |
| Hospital | Continuous | | | | | | | | | |
| utilization | | | | | | | | | | |

Metastatic ccRCC Disease/Response Assessments (Central)

| | | | | | | | | |
|---|---|---|---|---|---|---|---|---|
| CT scan ¹⁶ | X | X | X | X | X | X | X | X |
| Tumor biopsy ^{17, 18} | X | | | | | | | |

Laboratory Assessments (Local)

| | | | | | | | | | | |
|---|---|---|---|---|---|---|---|---|---|---|
| CBC w/ | X | X | X | X | X | X | X | X | X | X |
| differential | | | | | | | | | | |
| Serum | X ¹⁹ | X | X | X | X | X | X | X | X | X |
| chemistry¹⁹ | | | | | | | | | | |
| Coagulation | X | X | X | | | | | | | |
| parameters | | | | | | | | | | |
| Viral serology²⁰ | | | | | | | | | | |
| Lymphocyte | X | X | X | X | X | X | X | X | X | X |
| subsets²¹ | | | | | | | | | | |
| Ferritin, CRP, | X | X | X | | | | | | | |
| Triglyceride | | | | | | | | | | |

Biomarkers (Blood, Central)

| | | | | | | | | | | |
|---|---|---|---|---|---|---|---|---|---|---|
| CTX130 levels ²² | X | X | X | X | X | X | X | X | X | X |
| Cytokines ²⁴ | X | X | X | X | X | | | | | |
| BSAP, PINP²⁵ | X | | | X | X | | X | | | |
| Anti-CTX130 | X | | | X | X | | X | | | X |
| Ab | | | | | | | | | | |
| Cell-free DNA | X | | X | X | X | X | X | | X | X |
| Exploratory | X | X | X | X | X | X | X | X | X | X |
| biomarkers ²⁶ | | | | | | | | | | |

AE: adverse event;
BSAP: bone-specific alkaline phosphatase;
Cas9: CRISPR-associated protein 9;
CBC: complete blood count;
chemo: chemotherapy;
CNS: central nervous system;
CRISPR: clustered regularly interspaced short palindromic repeats;
CRP: C-reactive protein;
CRS: cytokine release syndrome;
CT: computed tomography;
d: day;
ECG: electrocardiogram;
EORTC: European Organization for Research and Treatment of Cancer;
FACT-G: functional assessment of cancer therapy-general;
FKSI-19: functional assessment of cancer therapy-kidney symptom index;
HBV: hepatitis B virus;
HCV: hepatitis C virus;
HIV: human immunodeficiency virus;
ICE: immune effector cell-associated encephalopathy;
LD: lymphodepleting;
M: month;
MRI: magnetic resonance imaging;
PINP: procollagen type 1 N propeptide;
PRO: patient-reported outcome;
TBNK: T, B, natural killer (NK) cells.
Note:
Baseline assessments are to be performed pre-CTX130 infusion on Day 1 unless otherwise specified; For samples tested centrally, refer to Laboratory Manual.
Note:
For both Part A and Part B, this study will allow for redosing of subjects with CTX130 per the redosing criteria discosed herein. All screening assessments must be repeated, including brain MRI. Subjects who are redosed should be followed per the schedule of assessments consistent with the initial dosing. The earliest time at which a subject could be redosed is 2 months after the initial or second CTX130 infusion.
¹ Screening assessments to be completed within 14 days after signing the informed consent form. Subjects will be allowed a one-time rescreening, which may take place within 3 months of the initial consent.
²Subjects should start LD chemotherapy within 7 days of study enrollment. After completion of LD chemotherapy, ensure washout period of at least 48 hours (but not greater than 7 days) before CTX130 infusion. Physical exam, weight, and coagulation laboratories are performed prior to LD chemotherapy. Vital signs, CBC, clinical chemistry, and AEs/concomitant medications should be assessed and recorded daily (i.e., 3 times) during LD chemotherapy.
3 CTX130 will be administered 48 hours to 7 days after completion of LD chemotherapy.
⁴Eligibility should be confirmed each time screening is completed. Eligibility should also be confirmed on the first day of LD chemotherapy, on day of CTX130 infusion. The eligibility should be confirmed after all assessments for that day are completed and before dosing.
⁵ Includes complete surgical and cardiac history.
⁶Includes assessment for signs and symptoms of GvHD: skin, oral mucosa, sclera, hands, and feet.
⁷ Includes blood pressure, heart rate, respiratory rate, pulse oximetry, and temperature.
⁸ Height at screening only.
⁹ For female subjects of childbearing potential. Assessed at local laboratory. Pregnancy tests are required at screening, within 72 hours of start of LD chemotherapy and at Ml/Day 28, M2/Day 56, and M3/Day 84. All tests will be serum pregnancy tests.
¹⁰Brain MRI to be performed at screening (i.e., within 28 days prior to CTX130 infusion).
¹¹ 12-lead ECG test should be conducted prior to LD chemotherapy and CTX130 infusion.
¹² On Day 1, prior to CTX130 administration. If CNS symptoms persist after Day 42, ICE assessment should continue to be performed approximately every 2 days until symptom resolution to Grade 1 or baseline.
¹³EORTC QLQ-C30, EQ-5D-5L, FKSI-19 questionnaires, and FACT-G. PROs should be completed at screening, pre dose on Day 1 and then Day 7, Day 15, Day 22, Day 28 post CTX130 infusion, and thereafter as specified in the schedule of assessment.
¹⁴ All concomitant medications will be collected up to 3 months post-CTX130 infusion. Afterwards, only select concomitant medications will be collected (i.e., immunomodulating agents, blood products, antitumor medications as well as hormones and growth factors).
¹⁵ Assessment of Safety, for the tabulated AE reporting requirements by study time period. Adverse events will be collected for enrolled subjects from the time of ICF signing until the end of study according to the AE reporting requirements for each time period of the study as described herein.
¹⁶ Baseline CT to be performed within 28 days prior to CTX130 infusion. CT for response assessment will be performed 6 weeks after CTX130 infusion (Day 42) and at Month 3, 6, 9, 12, 15, 18 and 24 post CTX130 infusion. Scans will be assessed locally and centrally for determination of objectives. Whenever possible, the same CT equipment and test parameters should be used. MRI will be performed where CT is contraindicated and after discussion with the medical monitor.
¹⁷ Biopsy to be performed at screening if post progression biopsy tissue is not available/acceptable, Day 7 + 2 days, and Day 42 ± 2 days after the dose of CTX130.
¹⁸ If relapse occurs on study, every attempt should be made to obtain biopsy of relapsed tumor and send to a central laboratory.
¹⁹ Creatinine is to be assessed more frequently between Days 1 and 28 to monitor for acute renal tubular damage: daily on Days 1-7, every other day between Days 8-15, and twice weekly until Day 28. If acute renal tubular damage is suspected, additional tests should be conducted including urine sediment analysis and fractional excretion of sodium in urine, and consultation by a nephrologist should be initiated.
²⁰ Includes HIV, HBV, and HCV at screening; however, historical results obtained within 60 days of enrollment may be used to determine eligibility.
²¹Lymphocyte subset assessment at screening, before start of first day of LD chemo, before CTX130 infusion, then all listed time points will be assessed at local laboratory. To include 6-color TBNK panel, or equivalent for T, B, and NK cells.
²² Samples for CTX130 levels should be collected from any lumbar puncture or tissue biopsy performed following CTX130 infusion. If CRS occurs, samples for assessment of CTX130 levels will be collected every 48 hours between scheduled visits until CRS resolves.
²³ Two samples are to be collected on Day 1: one pre-CTX130 infusion and another 20 minutes ± 5 min after the end of CTX130 infusion.
²⁴ Initial sample collection to occur at onset of symptoms. Additional cytokine samples should be collected every 12 hours(±5 hours) for the duration of CRS.
²⁵Samples are to be collected at the same time of day (±2 hours) on the specified collection days as disclosed herein.
²⁶ If CRS occurs, samples for assessment of exploratory biomarkers will be collected every 48 hours (±5 hours) between scheduled visits until CRS resolves. Samples for exploratory biomarkers should be collected from any lumbar puncture performed following CTX130 infusion as disclosed in this study.
²⁷An additional sample will be collected at screening for germ-line DNA extraction.
²⁸Prior to first day of LD chemotherapy only.

TABLE 22
Schedule of Assessments (Months 30-60).

| | M30 | M36 | M42 | M48 | M54 | M60 | Progressive | Secondary |
|---|---|---|---|---|---|---|---|---|
| Assessments | (±21 days) | (±21 days) | (±21 days) | (±21 days) | (±21 days) | (±21 days) | Disease | Follow-up ¹ |
| Vital signs ² | X | X | X | X | X | X | X | X |
| Physical exam | X | X | X | X | X | X | X | X |
| PRO ³ | | X | | X | | X | X | |
| Concomitant medications ⁴ | X | X | X | X | X | X | X | X |
| AEs ⁵ | X | X | X | X | X | X | X | X |
| Disease assessment ⁶ | X | X | X | X | X | X | X | |

Laboratory Assessments (Blood, Local)

| | | | | | | | | |
|---|---|---|---|---|---|---|---|---|
| CBC with differential | X | X | X | X | X | X | X | X |
| Serum chemistry | X | X | X | X | X | X | X | X |
| Lymphocyte subsets⁷ | X | X | X | X | X | X | X | |

Biomarkers (Blood, Central)

| | | | | | | | | |
|---|---|---|---|---|---|---|---|---|
| CTX130 persistence ⁸ | X | X | X | X | X | X | X | X |
| Anti-CTX130 antibody | | X | | X | | X | X | |
| Exploratory biomarkers | X | X | X | X | X | X | X | X |

AE: adverse event;
Cas9: CRISPR-associated protein 9;
CBC: complete blood count;
CRISPR: clustered regularly interspaced short palindromic repeats;
CT: computed tomography;
EORTC: European Organization for Research and Treatment of Cancer;
FACT-G: functional assessment of cancer therapy-general;
FKSI-19: functional assessment of cancer therapy-kidney symptom index;
M: month;
MRI: magnetic resonance imaging;
PRO: patient-reported outcome;
SCT: stem cell transplant;
TBNK: T, B, natural killer (NK) cells.
¹ Subjects with progressive disease or who undergo SCT will discontinue the normal schedule of assessments and attend annual study visits. Subjects who partially withdraw consent will undergo these procedures at minimum.
² Includes sitting blood pressure, heart rate, respiratory rate, pulse oximetry, and temperature.
³ EORTC QLQ-C30, EQ-5D-5L, FKSI-19 questionnaires, and FACT-G.
⁴ Only select concomitant medications will be collected.
⁵ Assessment of Safety, for the tabulated AE reporting requirements by study time period. AEs will be collected for enrolled subjects from the time of informed consent signing until the end of study according to the AE reporting requirements at each time period of the study, as described herin.
⁶ Disease assessment will consist of investigator review of physical exam, CBC, and clinical chemistry. Subjects with suspected malignancy will undergo CT (or possible MRI) imaging and/or a tissue biopsy to confirm relapse. Every attempt should be made to obtain a biopsy of the relapsed tumor in subjects who progress.
⁷Assessed at local laboratory. To include 6-color TBNK panel, or equivalent for T, B, and NK cells.
⁸ Samples for CTX130 levels should be sent to a central laboratory from any lumbar puncture or tissue biopsy performed following CTX130 infusion.

### 6.1 Subject Screening

6.1.1 Karnofsky Performance Status

Performance status is assessed at the time points outlined in Table 21 using the Karnofsky scale to determine the subject's general well-being and ability to perform activities of daily life, with scores ranging from 0 to 100. A higher score means better ability to carry out daily activities.

The Karnofsky performance status scale is shown in Table 23, and is used to determine performance status in the current study (Péus et al., (2013) BMC Med Inform Decis Mak., 13: 72.

TABLE 23
Karnofsky Performance Status Scale.

| | Karnofsky |
|---|---|
| Karnofsky Status | Grade |
| Normal, no complaints | 100 |
| Able to carry on normal activities; | 90 |
| Minor signs or symptoms of disease | |
| Normal activity with effort | 80 |
| Cares for self. Unable to carry on | 70 |
| normal activity or to do active work | |
| Requires occasional assistance, but | 60 |
| able to care for most of his needs | |
| Requires considerable assistance and | 50 |
| frequent medical care | |
| Disabled. Requires special care | 40 |
| and assistance | |
| Severely disabled. Hospitalization | 30 |
| indicated though death non imminent | |
| Very sick. Hospitalization necessary. | 20 |
| Active supportive treatment necessary | |
| Moribund | 10 |
| Dead | 0 |

6.1.2 Brain MRI

To rule out CNS metastasis, a brain MRI will be performed at screening (i.e., within 28 days prior to CTX130 infusion). Requirements for the acquisition, processing, and transfer of this MRI will be outlined in the Imaging Manual.

6.1.3 Echocardiogram A transthoracic cardiac echocardiogram (for assessment of left ventricular ejection fraction) will be performed and read by trained medical personnel at screening to confirm eligibility. In case of cardiac symptoms during CRS, medically appropriate assessment should be initiated in accordance with institutional guidelines.

6.1.4 Electrocardiogram

Twelve (12)-lead electrocardiograms (ECGs) are obtained during screening, prior to each LD chemotherapy on the first day of treatment, prior to CTX130 administration on Day 1, and on Day 42. QTc and QRS intervals are determined from ECGs. Additional ECGs may be obtained.

6.1.5 ccRCC Disease and Response Assessments

Disease evaluations are based on assessments in accordance with the RECIST v1.1 criteria (Eisenhauer et al., (2009) European Journal of Cancer 45, 228-247) and described herein, e.g.: Section 6.2. For efficacy analyses, disease outcome is graded using RECIST v1.1 response criteria. ccRCC disease and response evaluation should be conducted per the schedule in Table 21 and Table 22, and include the assessments described herein.

6.1.6 Radiographic Disease Assessment (CT or MRI)

Whenever possible, the same CT equipment and test parameters should be used. MRI is performed where CT is contraindicated and after discussion with the medical monitor.

Baseline CT to be performed at screening (i.e., within 28 days prior to CTX130 infusion), 6 weeks after CTX130 infusion (on Day 42), and at Month 3 (Day 84), 6, 9, 12, 15, 18, and 24 post-CTX130 infusion per the schedule of assessments in Table 21, per RECIST v1.1 (e.g.: Section 6.2), and as clinically indicated. Scans are assessed locally and centrally for determination of objectives.

CT scans should be acquired with 5 mm slices with no intervening gap (contiguous). Should a subject have a contraindication for CT IV contrast, a noncontrast CT of the chest and a contrast-enhanced magnetic resonance imaging (MRI) of the abdomen and pelvis may be obtained. MRIs should be acquired with slice thickness of 5 mm with no gap (contiguous). Every attempt should be made to image each subject using an identical acquisition protocol on the same scanner for all imaging time.

In addition, if a subject receives a fluorodeoxyglucose (FDG)-positron emission tomography (PET)/CT scan for reasons outside of the study, it is possible that the CT component of the scan may be used to assess disease response.

Whenever possible, the imaging modalities, machines, and scanning parameters used for radiographic disease assessment should be kept consistent during the study.

6.1.7 Tumor Biopsy

Subjects are required to undergo tumor biopsy at screening or, if a post-progression biopsy was performed within 3 months prior to enrollment and after the last systemic or targeted therapy, archival tissue may be provided. If archival tissue is of insufficient volume or quality to fulfill central laboratory requirements, a biopsy must be performed during screening (see disclosures in this Example).

Tumor biopsy will also be performed on Day 7 (+2 days; or as soon as clinically feasible) and Day 42 (±2 days). If a relapse occurs while a subject is on study, every attempt should be made to obtain biopsy of relapse tumor and send to a central laboratory.

Biopsies should come from measurable but nontarget lesions according to RECIST 1.1 analysis. When multiple biopsies are taken, efforts should be made to obtain them from similar tissues. Liver metastases are generally less desired. Bone biopsies and other decalcified tissues are not acceptable due to interference with downstream assays. This sample is analyzed for presence of CTX130 as well as tumor intrinsic and TME-specific biomarkers including analysis of DNA, RNA, protein and metabolites.

6.1.8 Patient-Reported Outcomes

Four patient-reported outcome (PRO) surveys are administered according to the schedules in Table 21 and Table 22: the European Organization for Research and Treatment of Cancer (EORTC) QLQ-C30, the EuroQol-5 Dimension-5 Level (EQ-5D-5L), the National Comprehensive Cancer Network (NCCN)-Functional Assessment of Cancer Therapy (FACT) Kidney Symptom Index (FKSI-19), and FACT-General (FACT-G) questionnaires. Questionnaires should be completed (self-administered in the language the subject is most familiar) before clinical assessments are performed.

The EORTC QLQ-C30 is a questionnaire designed to measure quality of life in cancer patients. It is composed of 5 multi-item functioning scales (physical, role, social, emotional, and cognitive function), 3 symptom scales (fatigue, nausea, pain) and additional single symptom items (financial impact, appetite loss, diarrhea, constipation, sleep disturbance, and quality of life). The EORTC QLQ-C30 is validated and has been widely used among cancer patients (Wisloff et al., (1996) Br J Haematol 92, 604-613; Wisloff and Hjorth, (1997) Br J Haematol 97, 29-37). It is scored on a 4-point scale (1=not at all, 2=a little, 3=quite a bit, 4=very much). The EORTC QLQ-C30 instrument also contains 2 global scales that use 7-point scale scoring with anchors (1=very poor and 7=excellent).

The EQ-5D-5L is a generic measure of health status and contains a questionnaire that assesses 5 domains, including mobility, self-care, usual activities, pain/discomfort, and anxiety/depression, plus a visual analog scale.

The NCCN-FACT FKSI-19 is designed as a brief symptom index for patients with advanced kidney cancer and includes perspectives of both clinicians and patients. The index includes 19 items within 3 subscales: disease-related symptoms (DRS), treatment side effects (TSE), and general function and well-being (FWB) (Rothrock et al., (2013) Value Health 16(5):789-96.).

The FACT-G questionnaire is designed to assess the health-related quality of life in patients undergoing cancer treatment. It is divided into physical, social/family, emotional, and functional domains (Cella et al., (1993) J Clin Oncol 11:570-79).

6.1.9 Immune Effector Cell-Associated Encephalopathy (ICE) Assessment

Neurocognitive assessment is performed using ICE assessment. The ICE assessment tool is a slightly modified version of the CARTOX-10 screening tool, which now includes a test for receptive aphasia (Neelapu et al., (2018) Nat Rev Clin Oncol 15, 47-62). ICE assessment examines various areas of cognitive function: orientation, naming, following commands, writing, and attention (Table 24A).

TABLE 24A
ICE Assessment.

| | | Maximum |
|---|---|---|
| Domain | Assessment | Score |
| Orientation | Orientation to year, | 4 points |
| | month, city, hospital | |
| Naming | Name 3 objects (e.g., point | 3 points |
| | to clock, pen, button) | |
| Following | Ability to follow commands | 1 point |
| command | (e.g., “Show me 2 fingers” | |
| | or “Close your eyes and | |
| | stick out your tongue”) | |
| Writing | Ability to write a standard | 1 point |
| | sentence (includes a noun | |
| | and verb) | |
| Attention | Ability to count backward | 1 point |
| | from 100 by 10 | |

ICE score is reported as the total number of points (0-10) across all assessments.

ICE assessment is performed at screening, before administration of CTX130 on Day 1, and on Days 2, 3, 5, 8, 42 and 56. If CNS symptoms persist beyond Day 42, ICE assessment should continue to be performed approximately every 2 days until resolution of symptoms to grade 1 or baseline. To minimize variability, whenever possible the assessment should be performed by the same research staff member who is familiar with or trained in administration of the ICE assessment tool.

6.1.10. Laboratory Tests

Laboratory samples will be collected and analyzed according to the schedule of assessment as disclosed in this study. Local laboratories meeting applicable local requirements (e.g., Clinical Laboratory Improvement Amendments) are utilized to analyze all tests listed in the following Table 24B.

TABLE 24B
Local Laboratory Tests

| | |
|---|---|
| CBC with differential | Hematocrit, hemoglobin, red blood cell count, white blood cell count, |
| | neutrophils, lymphocytes, monocytes, basophils, eosinophils, platelet count, |
| | absolute neutrophil count |
| Serum chemistry | ALT (SGPT), AST (SGOT), bilirubin (total and direct), albumin, alkaline |
| | phosphatase, bicarbonate, BUN, calcium, chloride, creatinine, eGFR, |
| | glucose, lactate dehydrogenase, magnesium, phosphorus, potassium, |
| | sodium, total protein, uric acid |
| Coagulation | PT, aPTT, international normalized ratio, fibrinogen |
| Viral serology ¹ | HIV-1, HIV-2, hepatitis C virus antibody and RNA, hepatitis B surface |
| | antigen, hepatitis B surface antibody, hepatitis B core antibody |
| Lymphocyte Subsets | 6-color TBNK panel or equivalent (T cells, B cells, and NK cells) |
| CRS/HLH monitoring | Ferritin, CRP, triglycerides |
| Serum pregnancy ² | Human chorionic gonadotropin (hCG) |

ALT: alanine aminotransferase; aPTT: activated partial thromboplastin time; AST: aspartateaminotransferase; BUN: blood urea nitrogen; CBC: complete blood count; CRP: C-reactive protein; CRS: cytokine release syndrome; eGFR: estimated glomerular filtration rate; HIV-1/-2: human immunodeficiency virus type 1 or 2; HLH: hemophagocytic lymphohistiocytosis; NK: natural killer; PT: prothrombin time; SGOT: serum glutamic oxaloacetic transaminase; SGPT: serum glutamic pyruvictransaminase; TBNK: T, B, and NK cells
¹ Historical viral serology results obtained within 60 days of enrollment may be used to determine eligibility.
² For females of childbearing potential only. Pregnancy test required at screening, within 72 hours of start of LD chemotherapy and at M1/Day 28, M2/Day 56, and M3/Day 84. All tests will be serum pregnancy tests.

6.2 Response Evaluation Criteria in Solid Tumors version 1.1 (RECIST v1.1)

The following is adapted from E. A. Eisenhauer, et al: New response evaluation criteria in solid tumors: Revised RECIST guideline (version 1.1). European Journal of Cancer 45 (2009) 228-247.

### Categorizing Lesions at Baseline

### Measurable Lesions

Lesions that can be accurately measured in at least one dimension.
Lesions with longest diameter twice the slice thickness and at least 10 mm or greater when assessed by CT or MRI (slice thickness 5-8 mm).
Lesions with longest diameter at least 20 mm when assessed by chest X-ray.
Superficial lesions with longest diameter 10 mm or greater when assessed by caliper.
Malignant lymph nodes with the short axis 15 mm or greater when assessed by CT.
NOTE: The shortest axis is used as the diameter for malignant lymph nodes, longest axis for all other measurable lesions.

### Non-Measurable Disease

Non-measurable disease includes lesions too small to be considered measurable (including nodes with short axis between 10 and 14.9 mm) and truly non-measurable disease such as pleural or pericardial effusions, ascites, inflammatory breast disease, leptomeningeal disease, lymphangitic involvement of skin or lung, clinical lesions that cannot be accurately measured with calipers, abdominal masses identified by physical exam that are not measurable by reproducible imaging techniques.
Bone disease: Bone disease is non-measurable with the exception of soft tissue components that can be evaluated by CT or MRI and meet the definition of measurability at baseline.
Previous local treatment: A previously irradiated lesion (or lesion subjected to other local treatment) is non-measurable unless it has progressed since completion of treatment.

### Normal Sites

Cystic lesions: Simple cysts should not be considered as malignant lesions and should not be recorded either as target or non-target disease. Cystic lesions thought to represent cystic metastases can be measurable lesions, if they meet the specific definition above. If non-cystic lesions are also present, these are preferred as target lesions.
Normal nodes: Nodes with short axis <10 mm are considered normal and should not be recorded or followed either as measurable or non-measurable disease.

### Recording Tumor Assessments

All sites of disease must be assessed at baseline. Baseline assessments should be done as close as possible prior to study start. For an adequate baseline assessment, all required scans must be done within 28 days prior to treatment and all disease must be documented appropriately. If baseline assessment is inadequate, subsequent statuses generally should be indeterminate.

### Target Lesions

All measurable lesions up to a maximum of 2 lesions per organ, 5 lesions in total, representative of all involved organs, should be identified as target lesions at baseline. Target lesions should be selected on the basis of size (longest lesions) and suitability for accurate repeated measurements. Record the longest diameter for each lesion, except in the case of pathological lymph nodes for which the short axis should be recorded. The sum of the diameters (longest for non-nodal lesions, short axis for nodal lesions) for all target lesions at baseline are the basis for comparison to assessments performed on study.
If two target lesions coalesce the measurement of the coalesced mass is used. If a large target lesion splits, the sum of the parts is used.
Measurements for target lesions that become small should continue to be recorded. If a target lesion becomes too small to measure, 0 mm should be recorded if the lesion is considered to have disappeared; otherwise a default value of 5 mm should be recorded.
NOTE: When nodal lesions decrease to <10 mm (normal), the actual measurement should still be recorded.

### Non-Target Disease

All non-measurable disease is non-target. All measurable lesions not identified as target lesions are also included as non-target disease. Measurements are not required but rather assessments are expressed as ABSENT, INDETERMINATE, PRESENT/NOT INCREASED, INCREASED. Multiple non-target lesions in one organ may be recorded as a single item on the case report form (e.g., ‘multiple enlarged pelvic lymph nodes’ or ‘multiple liver metastases’).

### Objective Response Status at Each Evaluation.

Disease sites must be assessed using the same technique as baseline, including consistent administration of contrast and timing of scanning. If a change needs to be made the case must be discussed with the radiologist to determine if substitution is possible. If not, subsequent objective statuses are indeterminate.

### Target Disease

Complete Response (CR): Complete disappearance of all target lesions with the exception of nodal disease. All target nodes must decrease to normal size (short axis <10 mm). All target lesions must be assessed.
Partial Response (PR): Greater than or equal to 30% decrease under baseline of the sum of diameters of all target measurable lesions. The short diameter is used in the sum for target nodes, while the longest diameter is used in the sum for all other target lesions. All target lesions must be assessed.
Stable: Does not qualify for CR, PR or Progression. All target lesions must be assessed. Stable can follow PR only in the rare case that the sum increases by less than 20% from the nadir, but enough that a previously documented 30% decrease no longer holds.
Objective Progression (PD): 20% increase in the sum of diameters of target measurable lesions above the smallest sum observed (over baseline if no decrease in the sum is observed during therapy), with a minimum absolute increase of 5 mm
Indeterminate. Progression has not been documented, and
   one or more target measurable lesions have not been assessed
   or assessment methods used were inconsistent with those used at baseline
   or one or more target lesions cannot be measured accurately (e.g., poorly visible unless due to being too small to measure)
   or one or more target lesions were excised or irradiated and have not reappeared or increased.

### Non-Target Disease

CR: Disappearance of all non-target lesions and normalization of tumor marker levels. All lymph nodes must be ‘normal’ in size (<10 mm short axis).
Non-CR/Non-PD: Persistence of any non-target lesions and/or tumor marker level above the normal limits.
PD: Unequivocal progression of pre-existing lesions. Generally the overall tumor burden must increase sufficiently to merit discontinuation of therapy. In the presence of SD or PR in target disease, progression due to unequivocal increase in non-target disease should be rare.
Indeterminate: Progression has not been determined and one or more non-target sites were not assessed or assessment methods were inconsistent with those used at baseline.

### New Lesions

The appearance of any new unequivocal malignant lesion indicates PD. If a new lesion is equivocal, for example due to its small size, continued assessment clarifies the etiology. If repeat assessments confirm the lesion, then progression should be recorded on the date of the initial assessment. A lesion identified in an area not previously scanned is considered a new lesion.

### Supplemental Investigations

If CR determination depends on a residual lesion that decreased in size but did not disappear completely, it is recommended the residual lesion be investigated with biopsy or fine needle aspirate. If no disease is identified, objective status is CR.
If progression determination depends on a lesion with an increase possibly due to necrosis, the lesion may be investigated with biopsy or fine needle aspirate to clarify status.

### Subjective Progression

Subjects requiring discontinuation of treatment without objective evidence of disease progression should not be reported as PD on tumor assessment CRFs. Every effort should be made to document objective progression even after discontinuation of treatment (see Table 25).

TABLE 25
Objective Response Status at each Evaluation.

| Target | Non-target | New | Objective |
|---|---|---|---|
| Lesions | Disease | Lesions | status |
| CR | CR | No | CR |
| CR | Non-CR/Non-PD | No | PR |
| CR | Indeterminate | No | PR |
| | or Missing | | |
| PR | Non-CR/Non-PD, | No | PR |
| | Indeterminate, | | |
| | or Missing | | |
| SD | Non-CR/Non-PD, | No | Stable |
| | Indeterminate, | | |
| | or Missing | | |
| Indeterminate | Non-PD | No | Indeterminate |
| or Missing | | | |
| PD | Any | Yes or No | PD |
| Any | PD | Yes or No | PD |
| Any | Any | Yes | PD |

CR: complete response; PD: progressive disease; PR: partial response.

For enrollment of patients with only non-target disease, the Table 26 is used.

TABLE 26
Objective Response Status at each Evaluation
for Patients with Non-Target Disease Only.

| Non-target | New | Objective |
|---|---|---|
| Disease | Lesions | status |
| CR | No | CR |
| Non-CR/Non-PD | No | Non-CR/Non-PD |
| Indeterminate | No | Indeterminate |
| Unequivocal | Yes or No | PD |
| progression | | |
| Any | Yes | PD |

### 7. Study Treatment

### 7.1 Lymphodepleting Chemotherapy

All subjects receive LD chemotherapy prior to the infusion of CTX130.

LD chemotherapy consists of:
Fludarabine 30 mg/m2 IV daily for 3 doses AND
Cyclophosphamide 500 mg/m2 IV daily for 3 doses.

Adult subjects with moderate impairment of renal function (creatinine clearance 50 70 ml/min/1.73 m²) should receive a reduced dose of fludarabine by at least 20% or in accordance with local prescribing information.

Both agents are started on the same day and administered for 3 consecutive days. Subjects should start LD chemotherapy within 7 days of study enrollment. LD chemotherapy must be completed at least 48 hours (but no more than 7 days) prior to CTX130 infusion.

LD chemotherapy is to be delayed if any of the following signs or symptoms are present:
Significant worsening of clinical status that increases the potential risk of AEs associated with LD chemotherapy.
Requirement for supplemental oxygen to maintain a saturation level of >91%.
New uncontrolled cardiac arrhythmia.
Hypotension requiring vasopressor support.
Active infection: Positive blood cultures for bacteria, fungus, or virus not responding to treatment, or negative culture but active infection is strongly suspected.
   Platelet count ≤100,000/mm3, absolute neutrophil count ≤1500/mm3, and hemoglobin (HgB) ≤9 g/dL without prior blood cell transfusion
Grade ≥2 acute neurological toxicity.

The goal of lymphodepletion is to allow for significant CAR T cell expansion following infusion. LD chemotherapy consisting of fludarabine and cyclophosphamide across different doses has been successfully utilized in several autologous CAR T-cell trials. The rationale for the use of LD chemotherapy is to eliminate regulatory T cells and other competing elements of the immune system that act as ‘cytokine sinks,’ enhancing the availability of cytokines such as interleukin 7 (IL-7) and interleukin 15 (IL-15) (Dummer et al., (2002) J Clin Invest 110, 185-192; Gattinoni et al., (2005) J Exp Med 202, 907-912). Additionally, it is postulated that naïve T cells begin to proliferate and differentiate into memory-like T cells when total numbers of naïve T cells are reduced below a certain threshold (Dummer et al., (2002) J Clin Invest 110, 185-192). The proposed LD chemotherapy dosage used in this protocol is consistent with doses used in registrational clinical trials of axicabtagene ciloleucel.

### 7.2 Administration of CTX130

CTX130 consists of allogeneic T cells modified with CRISPR-Cas9, resuspended in cryopreservative solution (CryoStor CS5), and supplied in a 6-ml infusion vial. A flat dose of CTX130 (based on % CAR⁺ T cells) is administered as a single IV infusion. The total dose may be contained in multiple vials. The infusion of each vial should occur within 20 minutes of thawing. Infusion should preferably occur through a central venous catheter. A leukocyte filter must not be used.

Prior to the start of CTX130 infusion, the site pharmacy must ensure that 2 doses of tocilizumab and emergency equipment are available for each specific subject treated. Subjects should be premedicated per the site standard of practice with oral acetaminophen (i.e., paracetamol or its equivalent per site formulary) and diphenhydramine hydrochloride IV or orally (or another H1-antihistamine per site formulary) approximately 30 to 60 minutes prior to CTX130 infusion. Prophylactic systemic corticosteroids should not be administered, as they may interfere with the activity of CTX130

CTX130 infusion can be delayed if any of the following signs or symptoms are present:
New active uncontrolled infection.
Worsening of clinical status compared to status prior to start of LD chemotherapy that places the subject at increased risk of toxicity.
Grade ≥2 acute neurological toxicity.

CTX130 is administered at least 48 hours (but no more than 7 days) after the completion of LD chemotherapy.

### 7.3 CTX130 Post-Infusion Monitoring

Following CTX130 infusion, subjects' vitals should be monitored every 30 minutes for 2 hours after infusion or until resolution of any potential clinical symptoms.

Subjects in Part A are hospitalized for a minimum of 7 days after CTX130 infusion. In both Parts A and B, subjects must remain in proximity of the investigative site (i.e., 1-hour transit time) for at least 28 days after CTX130 infusion. Management of acute CTX130-related toxicities should occur ONLY at the study site.

Subjects are monitored for signs of cytokine release syndrome (CRS), tumor lysis syndrome (TLS), graft versus host disease (GvHD), and other adverse events (AEs) according to the schedule of assessments (Table 21 and Table 22). Guidelines for the management of CAR T cell-related toxicities are described in Section 8. Subjects should remain hospitalized until CTX130-related nonhematologic toxicities (e.g., fever, hypotension, hypoxia, ongoing neurological toxicity) return to Grade 1. Subjects may remain hospitalized for longer periods if considered necessary by medical administrators.

### 7.4 Prior and Concomitant Medications

7.4.1 Allowed Medications and Procedures (Concomitant Treatments)

Necessary supportive measures for optimal medical care are given throughout the study, including IV antibiotics to treat infections, erythropoietin analogs, blood components, etc., except for prohibited medications described herein.

All concurrent therapies, including prescription and nonprescription medication, and medical procedures must be recorded from the date of signed informed consent through 3 months after CTX130 infusion. Beginning 3 months post-CTX130 infusion, only the following selected concomitant medications are collected: vaccinations, anti-cancer treatments (e.g., chemotherapy, radiation, immunotherapy), immunosuppressants (including steroids), and any investigational agents.

7.4.2 Prohibited/Restricted Medications and Procedures

The following medications are prohibited during certain periods of the study as specified below:
Within 28 days prior to enrollment and 3 months after CTX130 infusion
   Live vaccines
   Herbal medicine as part of traditional Chinese medicine or no-over-the-counter herbal remedies
Throughout the study until the start of new anticancer therapy
   Any immunosuppressive therapy unless recommended as described herein to treat CRS or immune effector cell associated neurotoxicity syndrome (ICANS) or if previously discussed with and approved by the medical monitor.
   Corticosteroid therapy at a pharmacologic dose (>10 mg/day of prednisone or equivalent doses of other corticosteroids) and other immunosuppressive drugs should be avoided after CTX130 administration unless medically indicated to treat new toxicity or as part of management of CRS or neurotoxicity associated with CTX130, as described herein.
   Any anti-cancer therapy (e.g., chemotherapy, immunotherapy, targeted therapy, radiation, or other investigational agents) other than LD chemotherapy prior to disease progression. Palliative radiation therapy for symptom management is permitted depending on extent, dose, and site(s), which should be defined and reported to the medical monitor for determination.
Prohibited Within the First Month After CTX130 Infusion
   Granulocyte-macrophage colony-stimulating factor (GM-CSF) due to the potential to worsen symptoms of CRS. Care should be taken with administration of granulocyte colony-stimulating factor (G-CSF) following CTX130 infusion, and the medical monitor must be consulted prior to administration.
Prohibited Within the First 28 Days After CTX130 Infusion (DLT Evaluation Period)
   Self-medication by the subject with antipyretics (e.g., acetaminophen, aspirin).

### 8. Toxicity Management

### 8.1 General Guidance

Prior to LD chemotherapy, infection prophylaxis (e.g., antiviral, antibacterial, antifungal agents) should be initiated according to institutional standard of care for ccRCC patients in an immunocompromised setting.

Subjects must be closely monitored for at least 28 days after CTX130 infusion. Significant toxicities have been reported with autologous CAR T cell therapies.

The following general recommendations are provided based on prior experience with autologous CD70 CAR T cell therapies:
Fever is the most common early manifestation of cytokine release syndrome (CRS); however, subjects may also experience weakness, hypotension, or confusion as first presentation.
Diagnosis of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, HLH, and TLS may occur at the same time following CAR T cell infusion. Subjects should be consistently monitored for signs and symptoms of all the conditions and managed appropriately.
Neurotoxicity may occur at the time of CRS, during CRS resolution, or following resolution of CRS. Grading and management of neurotoxicity are performed separately from CRS.
Tocilizumab must be administered within 2 hours from the time of order.

In addition to toxicities observed with autologous CAR T cells, signs of GvHD are monitored closely due to the allogeneic nature of CTX130.

The safety profile of CTX130 is continually assessed throughout the study.

### 8.2 Toxicity-Specific Guidance

8.2.1 CTX130 Infusion-Related Reactions

Infusion-related reactions have been reported in autologous CAR T cell trials, including transient fever, chills, and/or nausea, most commonly occurring within 12 hours after administration. CTX130 is formulated with CryoStor CS5, a well-established cryopreservant medium that contains 5% dimethyl sulfoxide (DMSO). Histamine release associated with DMSO can result in adverse effects such as nausea, vomiting, diarrhea, flushing, fevers, chills, headache, dyspnea, or rashes. In most severe cases, it can also cause bronchospasm, anaphylaxis, vasodilation and hypotension, and mental status changes.

If an infusion reaction occurs, acetaminophen (paracetamol) and diphenhydramine hydrochloride (or another H1 antihistamine) may be repeated every 6 hours after CTX130 infusion, as needed.

Nonsteroidal anti-inflammatory drugs (NSAIDs) may be prescribed, as needed, if the subject continues to have fever not relieved by acetaminophen. Systemic steroids should NOT be administered except in cases of life-threatening emergency, as this intervention may have a deleterious effect on CAR T cells.

8.2.2 Infection Prophylaxis and Febrile Reaction

Infection prophylaxis should be managed according to the institutional standard of care for ccRCC patients in an immunocompromised setting.

In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX130 infusion, appropriate cultures and medical management should be initiated. Additionally, consideration of CRS should be given in any instances of fever following CTX130 infusion within 28 days post-infusion.

Viral encephalitis (e.g., human herpes virus [HHV]-6 encephalitis) must be considered in the differential diagnosis for subjects who experience neurocognitive symptoms after receiving CTX130. A lumbar puncture (LP) is required for any Grade 3 or higher neurocognitive toxicity and is strongly recommended for Grade 1 and Grade 2 events. Whenever a lumbar puncture is performed, an infectious disease panel will review data from the following assessments (at a minimum) quantitative testing for HSV 1&2, Enterovirus, Human Parechovirus, VZV, CMV, and HHV-6. Lumbar puncture must be performed within 48 hours of symptom onset and results from the infectious disease panel must be available within 4 days of the LP in order to appropriately manage the subject.

8.2.3 Tumor Lysis Syndrome (TLS)

Subjects receiving CAR T cell therapy may be at increased risk of TLS, which occurs when tumor cells release their contents into the bloodstream, either spontaneously or in response to therapy, leading to the characteristic findings of hyperuricemia, hyperkalemia, hyperphosphatemia, hypocalcemia, and elevated blood urea nitrogen. These electrolyte and metabolic disturbances can progress to clinical toxic effects, including renal insufficiency, cardiac arrhythmias, seizures, and death due to multiorgan failure (Howard et al., 2011). TLS has been reported in hematomalignancies as well as solid tumors. Most solid tumors pose a low risk for TLS. It has been most frequently observed in patients with hematomalignancies, in particular leukemic forms such as ALL, acute myeloid leukemia, and CLL, which have a high (>5%) risk for TLS, and noncutaneous T cell lymphomas, particularly adult T cell leukemia/lymphoma and DLBCL (Coiffier et al., 2008). Additional risk factors include lactate dehydrogenase level higher than ULN, high tumor burden, and tumors with high replicative index. Patients with compromised renal function are also at elevated risk for developing TLS.

Subjects should be closely monitored for TLS via laboratory assessments and symptoms from the start of LD chemotherapy until 28 days following CTX130 infusion. Subjects at increased risk of TLS should receive prophylactic allopurinol (or a nonallopurinol alternative such as febuxostat) and/or rasburicase and increased oral/IV hydration during screening and before initiation of LD chemotherapy. Prophylaxis can be stopped after 28 days following CTX130 infusion or once the risk of TLS passes.

Sites should monitor and treat TLS as per their institutional standard of care, or according to published guidelines (Cairo and Bishop, (2004) Br J Haematol, 127, 3-11). TLS management, including administration of rasburicase, should be instituted promptly when clinically indicated.

8.2.4 Cytokine Release Syndrome (CRS)

CRS is a toxicity associated with immune therapies, including CAR T cells, resulting from a release of cytokines, in particular IL-6 and IL-1 (Norelli et al., (2018) Nat Med 24(6):739-748). CRS is due to hyperactivation of the immune system in response to CAR engagement of the target antigen, resulting in multicytokine elevation from rapid T cell stimulation and proliferation (Frey et al., (2014) Blood 124, 2296); Maude et al., (2014) Cancer J 20, 119-122). CRS has been observed in clinical trials irrespective of the antigen-targeted agents, including CD19−, BCMA−, CD123−, and mesothelin-directed CAR T cells, and anti-NY-ESO 1 and MART 1-targeted TCR-modified T cells (Frey et al., 2014; Hattori et al., 2019; Maude et al., 2018; Neelapu et al., 2017; Raje et al., 2019; Tanyi et al., 2017). CRS is a major toxicity reported with autologous CAR T cell therapy that has also been observed in early phase studies with allogeneic CAR T cell therapy (Benjamin et al., 2018).

The clinical presentation of CRS may be mild and be limited to elevated temperatures or can involve one or multiple organ systems (e.g., cardiac, gastrointestinal, respiratory, skin, central nervous) and multiple symptoms (e.g., high fevers, fatigue, anorexia, nausea, vomiting, rash, hypotension, hypoxia, headache, delirium, confusion). CRS may be life-threatening. Clinically, CRS can be mistaken for a systemic infection or, in severe cases, septic shock. Frequently the earliest sign is elevated temperature, which should prompt an immediate differential diagnostic work-up and timely initiation of appropriate treatment.

The goal of CRS management is to prevent life-threatening states and sequelae while preserving the potential for the anticancer effects of CTX130. Symptoms usually occur 1 to 14 days after autologous CAR T cell therapy in hematologic malignancies.

CRS should be identified and treated based on clinical presentation and not laboratory measurements. If CRS is suspected, grading should be applied according to the American Society for Transplantation and Cellular Therapy (ASTCT; formerly known as American Society for Blood and Marrow Transplantation, ASBMT) consensus recommendations (Table 27A; Lee et al., 2019), and management should be performed according to the recommendations in Table 27B, which are adapted from published guidelines (Lee et al., 2014; Lee et al., 2019). Accordingly, grading of neurotoxicity will be aligned with the ASTCT criteria for ICANS.

TABLE 27A
Grading of CRS according to the ASTCT consensus criteria (Lee et al., 2019)

| CRS | | | | |
|---|---|---|---|---|
| Parameter | Grade 1 | Grade 2 | Grade 3 | Grade 4 |
| Fever¹ | Temperature | Temperature | Temperature | Temperature |
| With | ≥38° C. | ≥38° C. | ≥38° C. | ≥38° C. |
| Hypotension | None | Not requiring | Requiring a | Requiring |
| | | vasopressors | vasopressor with | multiple |
| | | | or without | vasopressors |
| | | | vasopressin ² | (excluding |
| | | | | vasopressin) ² |
| And/or³ | None | Requiring low- | Requiring high- | Requiring positive |
| Hypoxia | | flow nasal | flow nasal | pressure (e.g., |
| | | cannula ⁴ or | cannula ⁴, | CPAP, BiPAP, |
| | | blow-by | facemask, | intubation, and |
| | | | nonrebreather | mechanical |
| | | | mask, or | ventilation) |
| | | | Venturi mask | |

ASTCT: American Society for Transplantation and Cellular Therapy; BiPAP: bilevel positive airway pressure; C.: Celsius; CPAP: continuous positive airway pressure; CRS: cytokine release syndrome
Note:
Organ toxicides associated with CRS may be graded according to CTCAE v5.0 but they do notinfluence CRS grading.
¹ Fever is defined as temperature ≥38° C. not attributable to any other cause. In patients who have CRSthen receive antipyretics or anticytokine therapy such astocilizumab or steroids, fever is no longerrequired to grade subsequent CRS severity. In this case, CRS grading is driven by hypotension and/orhypoxia.
² See Table 28 for information on high-dose vasopressors
³ CRS grade is determined by the more severe event: hypotension or hypoxia not attributable to anyother cause. For example, a patient with temperature of 39.5° C., hypotension requiring 1 vasopressor,and hypoxia requiring low-flow nasal cannula is classified as Grade 3 CRS.
⁴ Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute

TABLE 27B
Cytokine Release Syndrome Grading and Management Guidance.

| CRS | | | Hypotension |
|---|---|---|---|
| Severity ¹ | Tocilizumab | Corticosteroids | Management |
| Grade 1 | Tocilizumab ² may be | N/A | N/A |
| | considered per | | |
| | investigator’s | | |
| | discretion in | | |
| | consultation with the | | |
| | medical monitor. | | |
| Grade 2 | Administer | Manage per | Manage per |
| | tocilizumab 8 mg/kg | institutional | institutional |
| | IV over 1 hour (not to | guidelines if | guidelines |
| | exceed 800 mg).² | no improvement | |
| | Repeat tocilizumab | after initial | |
| | every 8 hours as | tocilizumab | |
| | needed if not | therapy. | |
| | responsive to IV fluids | Continue | |
| | or increasing | corticosteroids | |
| | supplemental oxygen. | use until the event | |
| | Limit to ≤3 doses in | is Grade ≤1, | |
| | a 24-hour period; | then taper | |
| | maximum total of | appropriately. | |
| | 4 doses. | | |
| Grade 3 | Per Grade 2 | Per Grade 2 | Manage per |
| | | | institutional |
| | | | guidelines |
| Grade 4 | Per Grade 2 | Per Grade 2 | Manage per |
| | If no response to | | institutional |
| | multiple doses of | | guidelines |
| | tocilizumab and | | |
| | steroids, consider | | |
| | using other | | |
| | anticytokine | | |
| | therapies | | |
| | (e.g., anakinra). | | |

CRS: cytokine release syndrome; IV: intravenously; N/A: not applicable.
¹ See Lee et al.. 2019.
²Refer to tocilizumab prescribing information.

TABLE 28
High-Dose Vasopressors in CRS Management.

| Pressor | Dose* | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent of ≥10 μg/min** |
| If on combination | Norepinephrine equivalent |
| vasopressors | of ≥20 μg/min** |
| (not vasopressin) | |

*All doses are required for ≥3 hours.
**VASST Trial vasopressor equivalent equation: norepinephrine equivalent dose = [norepinephrine(μg/min)] + [dopamine (μg/min)/2] + [epinephrine (μg/min)] + [phenylephrine (μg/min)/10].

Throughout the duration of CRS, subjects should be provided with supportive care consisting of antipyretics, IV fluids, and oxygen. Subjects who experience Grade ≥2 CRS should be monitored with continuous cardiac telemetry and pulse oximetry. For subjects experiencing Grade 3 CRS, consider performing an echocardiogram to assess cardiac function. For Grade 3 or 4 CRS, consider intensive care supportive therapy. The potential of an underlying infection in cases of severe CRS may be considered, as the presentation (e.g., fever, hypotension, hypoxia) is similar Resolution of CRS is defined as resolution of fever (temperature ≥38° C.), hypoxia, and hypotension (Lee et al., (2018) Biol Blood Marrow Transplant 25(4):625-638).

Hypotension and Renal Insufficiency

Hypotension and renal insufficiency have been reported with CAR T cell therapy and should be treated with IV administration of normal saline boluses according to institutional practice guidelines. Dialysis should be considered when appropriate.

8.2.5 Immune Effector Cell-Associated Neurotoxicity Syndrome (ICANS)

Neurotoxicity has been documented in subjects with B cell malignancies treated with autologous CAR T cell therapies. Therefore, subjects will be monitored for signs and symptoms of neurotoxicity associated with CAR T cell therapies in the current trial. Neurotoxicity may occur at the time of CRS, during the resolution of CRS, or following resolution of CRS, and its pathophysiology is unclear. The recent ASTCT (formerly known as ASBMT) consensus further defined ICANS as a disorder characterized by a pathologic process involving the CNS following any immune therapy that results in activation or engagement of endogenous or infused T cells and/or other immune effector cells (Lee et al., 2019). The pathophysiology of neurotoxicity remains unclear; however, it is postulated that it may be due to a combination of cytokine release, trafficking of CAR T into CSF, and increased permeability of the blood-brain barrier (June et al., 2018).

Signs and symptoms can be progressive and may include but are not limited to aphasia, altered level of consciousness, impairment of cognitive skills, motor weakness, seizures, and cerebral edema. ICANS grading (Table 29) was developed based on CAR T cell-therapy-associated TOXicity (CARTOX) working group criteria used previously in autologous CART cell trials (Neelapu et al., (2018) Nat Rev Clin Oncol 15, 47-62). ICANS incorporates assessment of level of consciousness, presence/absence of seizures, motor findings, presence/absence of cerebral edema, and overall assessment of neurologic domains by using a modified tool called the ICE (immune effector cell-associated encephalopathy) assessment tool (Table 24).

Evaluation of any new onset neurotoxicity should include a neurological examination (including ICE assessment tool, Table 24), brain magnetic resonance imaging (MRI), and examination of the CSF as clinically indicated. For lumbar punctures performed during neurotoxicity, CSF samples should be sent to a central laboratory for cytokine analysis and for presence of CTX130. Excess sample (if available) will be stored for exploratory research. Infectious etiology should be ruled out by performing a lumbar puncture whenever possible (especially for subjects with Grade 3 or 4 ICANS). If a brain MRI is not possible, all subjects should receive a non-contrast computed tomography (CT) scan to rule out intracerebral hemorrhage. Electroencephalogram should also be considered as clinically indicated. Endotracheal intubation may be needed for airway protection in severe cases.

Non-sedating, anti-seizure prophylaxis (e.g., levetiracetam) may be considered, especially in subjects with a history of seizures, for at least 28 days following CTX130 infusion or upon resolution of neurological symptoms (unless the antiseizure medication is contributing to the detrimental symptoms). Subjects who experience Grade ≥2 ICANS should be monitored with continuous cardiac telemetry and pulse oximetry. For severe or life-threatening neurologic toxicities, intensive care supportive therapy should be provided. Neurology consultation should always be considered. Monitor platelets and for signs of coagulopathy and transfuse blood products appropriately to diminish risk of intracerebral hemorrhage. Table 29 provides neurotoxicity grading and Table 30 provides management guidance.

TABLE 29
ICANS Grading.

| Neurotoxicity | | | | |
|---|---|---|---|---|
| Domain | Grade 1 | Grade 2 | Grade 3 | Grade 4 |
| ICE score ¹ | 7-9 | 3-6 | 0-2 | 0 (subject is |
| | | | | unarousable and |
| | | | | unable to undergo |
| | | | | ICE assessment) |
| Depressed | Awakens | Awakens | Awakens only to | Subject is unarousable |
| level of | spontaneously | to voice | tactile stimulus | or requires vigorous |
| consciousness ² | | | | or repetitive tactile |
| | | | | stimuli to arise; |
| | | | | stupor or coma |
| Seizure | N/A | N/A | Any clinical seizure, | Life-threatening |
| | | | focal or generalized, | prolonged seizure |
| | | | that resolves rapidly, | (>5 min) or repetitive |
| | | | or nonconvulsive | clinical or electrical |
| | | | seizures on EEG | seizures without |
| | | | that resolve with | return to baseline |
| | | | intervention | in between |
| Motor | N/A | N/A | N/A | Deep focal motor |
| findings ³ | | | | weakness such as |
| | | | | hemiparesis or |
| | | | | paraparesis |
| Elevated | N/A | N/A | Focal/local | Diffuse cerebral |
| ICP/cerebral | | | edema on | edema on |
| edema | | | neuroimaging ⁴ | neuroimaging, |
| | | | | decerebrate or |
| | | | | decorticate posturing, |
| | | | | cranial nerve VI |
| | | | | palsy, papilledema, |
| | | | | or Cushing’s triad |

CTCAE: Common Terminology Criteria for Adverse Events; EEG: electroencephalogram; ICANS: immune effector cell-associated neurotoxicity syndrome; ICE: immune effector cell-associatedencephalopathy (assessment tool); ICP: intracranial pressure; N/A: not applicable.
Note:
ICANS grade is determined by the most severe event (ICE score, level of consciousness, seizure, motor findings, raised ICP/cerebral edema) not attributable to any other cause.
¹ A subject with an ICE score of 0 may be classified as Grade 3 ICANS if awake with global aphasia,but a subject with an ICE score of 0 may be classified as Grade 4 ICANS if unarousable (Table 24Afor ICE assessment tool).
² Depressed level of consciousness should be attributable to no other cause (e.g., sedating medication).
³ Tremors and myoclonus associated with immune effector therapies should be graded according to CTCAE v5.0 but do not influence ICANS grading.

TABLE 30
ICANS Management Guidance.

| Severity | Management |
|---|---|
| Grade 1 | Provide supportive care per institutional practice. |
| Grade 2 | Consider administering dexamethasone 10 mg IV every 6 hours (or equivalent |
| | methylprednisolone) unless subject already on equivalent dose of steroids for CRS. |
| | Continue dexamethasone use until event is grade ≤1, then taper over 3 days. |
| Grade 3 | Administer dexamethasone 10 mg IV every 6 hours, unless subject already on |
| | equivalent dose of steroids for CRS. |
| | Continue dexamethasone use until event is grade ≤1, then taper over 3 days. |
| Grade 4 | Administer methylprednisolone 1000 mg IV per day for 3 days; if improves, then |
| | manage as above. |

CRS: cytokine release syndrome; ICANS: immune effector cell-associated neurotoxicity syndrome; IV: intravenously.

Headache, which may occur in a setting of fever or after chemotherapy, is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

8.2.6 Hemophagocytic Lymphohistiocytosis (HLH)

HLH has been reported after treatment with autologous CAR T cells (Barrett et al., (2014) Curr Opin Pediatr, 26, 43-49; Maude et al., (2015) Blood, 125, 4017-4023; Porter et al., (2015) Sci Transl Med, 7, 303ra139; Teachey et al., (2013) Blood, 121, 5154-5157). HLH is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP). Neurologic findings have also been observed (Jordan et al., (2011) Blood, 118, 4041-4052; La Rosée, (2015) Hematology Am Soc Hematol Educ Program, 190-196.

CRS and HLH may possess similar clinical syndromes with overlapping clinical features and pathophysiology. HLH likely occurs at the time of CRS or as CRS is resolving. HLH should be considered if there are unexplained elevated liver function tests or cytopenias with or without other evidence of CRS. Monitoring of CRP and ferritin may assist with diagnosis and define the clinical course. Where feasible, excess bone marrow samples should be sent to a central laboratory following routine practice.

If HLH is suspected:
Frequently monitor coagulation parameters, including fibrinogen. These tests may be done more frequently than indicated in the schedule of assessments, and frequency should be driven based on laboratory findings.
Fibrinogen should be maintained ≥100 mg/dL to decrease risk of bleeding.
Coagulopathy should be corrected with blood products.
Given the overlap with CRS, manage according to Grade 3 CRS with appropriate monitoring intensity per CRS treatment guidance in Table 27B. Follow institutional guidelines for additional treatment of HLH.

8.2.7 Cytopenias

Grade 3 neutropenia and thrombocytopenia, at times lasting more than 28 days after CAR T cell infusion, have been reported in subjects treated with autologous CAR T cell products (Kymriah US prescribing information [USPI], 2017; Raje et al., (2019) N Engl J Med 380, 1726-37; Yescarta USPI, 2017). Therefore, subjects receiving CTX130 should be monitored for such toxicities and appropriately supported. Monitor platelets and for signs of coagulopathy and transfuse blood products appropriately to diminish risk of hemorrhage. Consideration should be given to antimicrobial and antifungal prophylaxis for any subject with prolonged neutropenia.

Due to the transient expression of CD70 on activated T and B lymphocytes, opportunistic infection such as viral reactivation may occur, which should be considered when clinical symptoms arise.

During dose escalation, G-CSF may be considered in cases of Grade 4 neutropenia post-CTX130 infusion. During cohort expansion G-CSF may be administered cautiously per healthcare practitioner's discretion.

8.2.8 Graft vs Host Disease (GvHD)

GvHD is seen in the setting of allogeneic HSCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic HSCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser and Blazar, (2017) N Engl J Med, 377, 2167-2179).

To support the proposed clinical study, a nonclinical Good Laboratory Practice (GLP)-compliant GvHD and tolerability study was performed in immunocompromised mice treated at 2 IV doses: a high dose of 4×10⁷ CTX130 cells per mouse (approximately 1.6×10⁹ cells/kg) and a low dose of 2×10⁷ cells per mouse (approximately 0.8×10⁹ cells/kg). Both dose levels exceed the proposed highest clinical dose by more than 10-fold when normalized for body weight. No mice treated with CTX130 developed fatal GvHD during the course of the 12-week study. At necropsy, mononuclear cell infiltration was observed in some animals in the mesenteric lymph node and the thymus Minimal to mild perivascular inflammation was also observed in the lungs of some animals. These findings are consistent with mild GvHD but did not manifest in clinical symptoms in these mice.

Further, due to the specificity of CAR insertion at the TRAC locus, it is highly unlikely for a T cell to be both CAR+ and TCR+. Remaining TCR+ cells are removed during the manufacturing process by immunoaffinity chromatography on an anti-TCR antibody column to achieve ≤0.4% TCR+ cells in the final product. A dose limit of 1×10⁵ TCR+ cells/kg is imposed for all dose levels. This limit is based on published reports on the number of allogeneic cells capable of causing severe GvHD during SCT with haploidentical donors (Bertaina et al., (2014) Blood, 124, 822-826). Through this specific editing, purification, and strict product release criteria, the risk of GvHD following CTX130 should be low, although the true incidence is unknown. However, given that CAR T cell expansion is antigen-driven and is likely occur only in TCR− cells, it is unlikely that the number of TCR+ cells would appreciably increase above the number infused.

Diagnosis and grading of GvHD should be based on published criteria (Harris et al., (2016) Biol Blood Marrow Transplant, 22, 4-10), as outlined in Table 31.

TABLE 31
Criteria for Grading Acute GvHD.

| | | Liver | | Lower GI | |
|---|---|---|---|---|---|
| | Skin | (bilirubin | | (stool | |
| Stage | (active erythema only) | mg/dL) | Upper GI | output/day) | |
| 0 | No active (erythematous) | <2 | No or | <500 | ml/day or |
| | GvHD rash | | intermittent | <3 | episodes/day |
| | | | nausea, | | |
| | | | vomiting, | | |
| | | | or anorexia | | |
| 1 | Maculopapular rash | 2-3 | Persistent | 500-999 | ml/day or |
| | <25% BSA | | nausea, | 3-4 | episodes/day |
| | | | vomiting, | | |
| | | | or anorexia | | |
| 2 | Maculopapular rash | 3.1-6 | — | 1000-1500 | ml/day or |
| | 25-50% BSA | | | 5-7 | episodes/day |
| 3 | Maculopapular rash | 6.1-15 | — | >1500 | ml/day or |
| | >50% BSA | | | >7 | episodes/day |

| | | | | |
|---|---|---|---|---|
| 4 | Generalized erythroderma | >15 | — | Severe abdominal pain |
| | (>50% BSA) plus | | | with or without ileus, or |
| | bullous formation | | | grossly bloody stool |
| | and desquamation | | | (regardless of stool |
| | >5% BSA | | | volume) |

BSA: body surface area; GI: gastrointestinal; GvHD: graft versus host disease.

Overall GvHD grade can be determined based on most severe target organ involvement.
Grade 0: No stage 1-4 of any organ.
Grade 1: Stage 1-2 skin without liver, upper GI, or lower GI involvement.
Grade 2: Stage 3 rash and/or stage 1 liver and/or stage 1 upper GI and/or stage 1 lower GI.
Grade 3: Stage 2-3 liver and/or stage 2-3 lower GI, with stage 0-3 skin and/or stage 0-1 upper GI.
Grade 4: Stage 4 skin, liver, or lower GI involvement, with stage 0-1 upper GI.

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible.

Recommendations for management of acute GvHD are outlined in Table 32. To allow for intersubject comparability at the end of the trial, these recommendations can be followed except in specific clinical scenarios in which following them could put the subject at risk.

TABLE 32
Acute GvHD Management

| Grade | Management |
|---|---|
| 1 | Skin: Topical steroids or immunosuppressants; |
| | if stage 2: prednisone 1 mg/kg (or equivalent dose). |
| 2-4 | Initiate prednisone 2 mg/kg daily (or equivalent dose). |
| | IV form of steroid such as methylprednisolone |
| | should be considered if there are concerns |
| | with malabsorption. |
| | Steroid taper may begin after improvement |
| | is seen after ≥3 days of steroids. |
| | Taper should be 50% decrease of total daily |
| | steroid dose every 5 days. |
| | GI: In addition to steroids, start anti-diarrheal |
| | agents per standard practice. |

GI: gastrointestinal; IV: intravenous.

Decisions to initiate second-line therapy should be made sooner for subjects with more severe GvHD. For example, secondary therapy may be indicated after 3 days with progressive manifestations of GvHD, after 1 week with persistent Grade 3 GvHD, or after 2 weeks with persistent Grade 2 GvHD. Second-line systemic therapy may be indicated earlier in subjects who cannot tolerate high-dose glucocorticoid treatment (Martin et al., (2012) Biol Blood Marrow Transplant, 18, 1150-1163). Choice of secondary therapy and when to initiate can be based on clinical judgment and local practice.

Management of refractory acute GvHD or chronic GvHD can be per institutional guidelines. Anti-infective prophylaxis measures should be instituted per local guidelines when treating subjects with immunosuppressive agents (including steroids).

8.2.9. On Target Off-Tumor Toxicities

Activity of CTX130 Against Activated T and B Lymphocytes, Dendritic Cells

Activated T and B lymphocytes express CD70 transiently and dendritic cells, as well as thymic epithelial cells, express CD70 to a certain degree. Thus, these cells might become a target for activated CTX130. Management of infections and cytopenias is disclosed herein.

Activity of CTX130 Against Osteoblasts

Activity of CTX130 was detected in nonclinical studies in cell culture of human primary osteoblasts. Hence, bone turnover will be monitored via calcium levels as well as 2 osteoblast-specific markers, amino-terminal propeptide of type I procollagen (PINP) and bone-specific alkaline phosphatase (BSAP), which are considered the most useful markers in the assessment of bone formation (Fink et al., 2000). Standardized assays for assessment of both markers in serum are available. The concentration of these peptide markers reflects the activity of osteoblasts and the formation of new bone collagen.

PINP and BSAP will be measured through a central laboratory assessment at screening, baseline, Days 7, 15, 22, and 28, and Months 3, 6, and 12 of the study as disclosed herein. Samples are to be collected at the same time of day (±2 hours) on the specified collection days because of the strong effect of circadian rhythm on bone turn over.

Activity of CTX130 Against Renal Tubular-Like Epithelium

Activity of CTX130 against renal tubular-like epithelial cells was detected in nonclinical studies of CTX130 in primary human kidney epithelium. Hence, subjects should be monitored for acute tubular damage by monitoring for an increase in serum creatinine of at least 0.3 mg/dL (26.5 μmon) over a 48-hour period and/or ≥1.5 times the baseline value within the previous 7 days. Serum creatinine will be assessed daily for the first 7 days post-CTX130 infusion, every other day between Days 8 through 15 of treatment, and then twice weekly until Day 28 as disclosed herein. If acute renal tubular damage is suspected, additional tests should be conducted including urine sediment analysis and fractional excretion of sodium in urine, and consultation by a nephrologist should be initiated.

8.2.10. Uncontrolled T Cell Proliferation

Upon recognition of target tumor antigen in vivo activation and expansion has been observed with CAR T cells (Grupp et al NEJM 2013). Autologous CAR T cells have been detected in peripheral blood, bone marrow, cerebrospinal fluid, ascites and other compartments (Badbaran et al Cancer 2020). If a subject develops signs of uncontrolled T cell proliferation, a sample from the clinical investigation should be submitted to the central laboratory for haplotyping to determine the origin of T cells.

### 9. Assessment of Safety

### 9.1 Definition of Adverse Event Parameters

9.1.1 Adverse Events

The International Conference on Harmonisation (ICH) Guideline for Good Clinical Practice (GCP) E6(R2) defines an AE as:

“Any untoward medical occurrence in a patient or clinical investigation subject administered a pharmaceutical product and which does not necessarily have a causal relationship with this treatment. An AE can therefore be any unfavorable and unintended sign (including an abnormal laboratory finding, for example), symptom or disease temporally associated with the use of a medicinal (investigational) product whether or not considered related to the medicinal (investigational) product.”

Additional criteria defining an AE also include any clinically significant worsening in the nature, severity, frequency, or duration of a subject's pre-existing condition. Adverse events can occur before, during or after treatment and can be either treatment-emergent (i.e., occurring post-CTX130 infusion) or nontreatment emergent. A nontreatment-emergent AE is any new sign or symptom, disease, or other untoward medical event that occurs after written informed consent has been obtained but before the subject has received CTX130.

Elective or pre-planned treatment or medical/surgical procedures (that was scheduled prior to the subject being enrolled into the study) for a documented pre-existing condition that did not worsen from baseline is not considered an AE (serious or nonserious). However, an untoward medical event occurring during the prescheduled elective procedure or routinely scheduled treatment should be recorded as an AE or SAE. Hospitalization for study treatment infusions or precautionary measures per institutional policy or as define in this study protocol are not considered AEs. Furthermore, if a subject has a planned hospitalization following CTX130 infusion, prolongation of that hospitalization for observation alone should not be reported as an SAE, unless it is associated with a medically significant event that meets other SAE criteria.

9.1.1.1 Abnormal Laboratory Findings

Abnormal laboratory findings considered to be clinically significant and should be reported as an adverse event. Whenever possible, these should be reported as a clinical diagnosis rather than the abnormal value itself. Abnormal laboratory results without clinical significance are not required to be recorded as AEs.

9.1.1.2 Disease Progression

Disease progression is an outcome and should not be reported as an AE. If a subject requires hospitalization or an intervention qualifying the AE as serious, the symptom should be reported as an SAE (e.g., spleen rupture due to local progression).

9.1.2 Serious Adverse Events

A serious adverse event (SAE) is any untoward medical occurrence that at any dose:
Results in death.
Is life-threatening.

This definition implies that the subject is at immediate risk of death from the event as it occurred. It does not include an event that, had it occurred in a more severe form, might have caused death.
Requires inpatient hospitalization or prolongation of existing hospitalization.

In general, hospitalization signifies that the subject has been at the hospital or emergency ward (usually involving at least an overnight stay) for observation and/or treatment that would not have been appropriate in an outpatient setting.
Results in persistent or significant disability/incapacity.
Results in a congenital anomaly/birth defect.
Other important/significant medical events

Medical and scientific judgment should be exercised in deciding whether expedited reporting is appropriate in other situations, such as important medical events that may not be immediately life-threatening or result in death or hospitalization but may jeopardize the subject or may require intervention to prevent one of the other outcomes listed in the definition above.

9.1.3 Adverse Events of Special Interest

An AESI (serious or non-serious) is one of scientific and medical concern specific to the product or program, for which ongoing monitoring and rapid communication can be appropriate.

Based on the reported clinical experience of autologous CAR T cells considered to be in the same pharmacological class, the following are identified as adverse events of special interest (AESIs):

1. CTX130 infusion-related reactions.

2. Grade ≥3 infections and infestations

3. Tumor lysis syndrome (TLS).

4. Cytokine release syndrome (CRS).

5. Immune effector cell associated neurotoxicity syndrome (ICANS).

6. Hemophagocytic lymphohistiocytosis (HLH).

7. Graft versus host disease (GvHD).

8. Uncontrolled T cell proliferation

In addition to the AESIs listed above, any new autoimmune disorder that the investigator determines is possibly related or related to CTX130 should be reported any time after CTX130 infusion.

### 9.2 Assessment of Adverse Events

9.2.1 Assessment of Causality

The relationship between each AE and CTX130, LD chemotherapy, and any protocol-mandated study procedure (all assessed individually) shall be assessed. The following shall be considered: (1) the temporal association between the timing of the event and administration of the treatment or procedure, (2) a plausible biological mechanism, and (3) other potential causes of the event (e.g., concomitant therapy, underlying disease) when making their assessment of causality.

The assessment of relationship is made based on the following definitions:
Related: There is a clear causal relationship between the study treatment or procedure and the AE.
Possibly related: There is some evidence to suggest a causal relationship between the study treatment or procedure and the AE, but alternative potential causes also exist.
Not related: There is no evidence to suggest a causal relationship between the study treatment or procedure and the AE.

If the relationship between the AE/SAE and the CTX130 is determined to be “possible,” the event is considered related to the CTX130 for the purposes of regulatory reporting.

An event is considered “not related” to use of the CTX130 if any of the following tests are met:
An unreasonable temporal relationship between administration of the CTX130 and the onset of the event (e.g., the event occurred either before, or too long after administration of the IP for the AE to be considered product-related).
A causal relationship between the CTX130 and the event is biologically implausible.
A clearly more likely alternative explanation for the event is present (e.g., typical adverse reaction to a concomitant drug and/or typical disease-related event).

Individual AE/SAE reports are considered “related” to use of the IP if the “not related” criteria are not met. If an SAE is assessed to be not related to any study intervention, an alternative etiology must be provided in the case report form (CRF).

9.2.1.1 Relationship to Protocol Procedures and/or Other Etiologies

An assessment of relationship of SAEs to protocol procedures may be provided, if an SAE is determined to be not related to treatment with CTX130 or LD Chemotherapy. An alternate etiology on the SAE Report Form shall be provided based on the criteria defined below:
Protocol-related Procedure/Intervention: The event occurred as a result of a procedure or intervention required during the study (e.g., blood collection, washout of an existing medication) for which there is no alternative etiology present in the subject's medical record. This is applicable to non-treatment emergent SAEs (i.e., SAEs that occur prior to the administration of CTX130) as well as treatment emergent SAEs.

9.2.2 Assessment of Severity Severity are graded according to the NCI CTCAE 5.0, except for CRS, ICANS, and GvHD, which are graded according to the criteria in Table 27, Table 29, and Table 31, respectively. The determination of severity for events where CTCAE grade or protocol-specified criteria are not available should be made based upon medical judgement (and documented in the CRF) using the severity categories of grades 1 to 5 described in Table 33.

9.2.3 Adverse Event Outcome

The outcome of an AE or SAE classified and reported as follows:
Fatal.
Not recovered/not resolved.
Recovered/resolved.
Recovered/resolved with sequelae.
Recovering/resolving.
Unknown

When recording and reporting death and fatal/grade 5 events, note that death is a subject outcome and fatal is an event outcome and should describe the SAE which was the cause of death. Subjects withdrawn from the study because of AEs are followed until the outcome is determined.

TABLE 33A
Adverse Event Severity

| | |
|---|---|
| Grade 1 | Mild; asymptomatic or mild symptoms; clinical or |
| | diagnostic observations only; intervention not indicated |
| Grade 2 | Moderate; minimal, local, or noninvasive intervention |
| | indicated; limiting age-appropriate instrumental ADL ¹ |
| Grade 3 | Severe or medically significant but not |
| | immediately life-threatening; |
| | hospitalization or prolongation of hospitalization |
| | indicated; disabling; |
| | limiting self-care ADL ² |
| Grade 4 | Life-threatening consequences; urgent |
| | intervention indicated |
| Grade 5 | Death related to AE |

ADL: Activities of Daily Living; AE: adverse event.
¹ Instrumental ADL refer to preparing meals, shopping for groceries or clothes, using the telephone, managing money, etc.
² Self-care ADL refer to bathing, dressing and undressing, feeding self, using the toilet, taking medications, and not bedridden.

See also Tables 27A, 27B, 29, and 31, and adverse event grading criteria for, e.g., CRS, ICANS, and GvHD disclosed herein.

### 10. Stopping Rules and Study Termination

### 10.1 Stopping Rules for Trial

The study may be paused if 1 or more of the following events occur:
Life-threatening (Grade 4) toxicity attributable to CTX130 that is unmanageable, unexpected, and unrelated to LD chemotherapy.
Death related to CTX130 within 30 days of infusion.
After at least 15 subjects have received CTX130, occurrence of Grade ≥2 GvHD that is steroid-refractory in >20% of the subjects.
After at least 15 subjects have been enrolled, determination of unexpected, clinically significant or unacceptable risk to subjects that occurred in >35% of the subjects (e.g., Grade 3 neurotoxicity not resolving within 7 days to Grade ≤2).
New malignancy (distinct from recurrence/progression of previously treated malignancy).

Part B (cohort expansion) is a single-arm study conducted using an optimal Simon 2 stage design. In the first stage, 22 subjects are to be treated with CTX130. If ≥7 subjects achieve an objective response (CR or PR) post-CTX130 infusion, it may be decided to expand enrollment to include an additional 48 treated subjects (71 total) in the second stage. If the decision is made to end the trial after the first stage, enrollment can be suspended, all available data are reviewed, and health authorities are notified as required.

In the event enrollment is permanently suspended, subjects who are already enrolled in the study may not proceed with LD chemotherapy and CTX130 infusion. Subjects who have already been treated with CTX130 remain in the study and continue to be followed per the study protocol or are required to transition to a long-term safety follow-up study.

### 10.2 Stopping Rules for Individual Subjects

Stopping rules for individual subjects are as follows:
Any medical condition that would put the subject at risk during continuing study-related treatments or follow-up.
If a subject is found not to have met eligibility criteria or has a major protocol deviation before the start of LD chemotherapy.

### 10.3 End of Study Definition

The end of the study is defined as the time at which the last subject completes the Month 60 visit (the last protocol-defined assessment), or, is considered lost to follow-up, withdraws consent, or dies.

### 10.4 Study Termination

This study may be discontinued at any time due to safety concerns, failure to meet expected enrollment goals, and/or administrative reasons. In the event this study is terminated early, subjects who have received CTX130 are required to participate in a separate long-term follow-up study for up to 15 years post-CTX130 infusion.

### 11. Statistical Methods

### 11.1 General Methods

Study data is summarized for disposition, demographic and baseline characteristics, safety, and clinical antitumor activity.

Categorical data is summarized by frequency distributions (number and percentages of subjects) and continuous data will be summarized by descriptive statistics (mean, standard deviation [SD], median, minimum, and maximum).

Subjects treated during the dose escalation phase will be pooled with those receiving the same dose of CTX130 during the expansion phase, unless otherwise specified. All summaries, listings, figures, and analyses will be performed by dose level.

Primary analysis time is defined as when 71 subjects in Part B have completed the 3-month disease response assessment, or are lost to follow-up, withdraw from the study, or die, whichever occurs first (defined in full analysis set [FAS]). The study data will be analyzed and reported in the primary clinical study report (CSR) based on primary analysis time. Additional data cumulated from primary analysis time to end of study will be reported. Full details of statistical analyses will be specified in the statistical analysis plan (SAP).

### 11.2 Study Objectives and Hypotheses

The primary objective of Part A is to assess the safety of escalating doses of CTX130 in subjects with unresectable or metastatic ccRCC.

The primary objective of Part B is to assess the efficacy of CTX130 in subjects with unresectable or metastatic ccRCC as measured by ORR according to RECIST v1.1.

### 11.3 Study Endpoints

11.3.1 Primary Endpoints

Part A (Dose Escalation): The incidence of dose-limiting toxicities (DLTs), and definition of RPBD.

Part B (Cohort Expansion): The objective response rate (ORR) defined as complete response (CR)+partial response (PR) according to the Response Evaluation Criteria in Solid Tumors (RECIST 1.1).

11.2.2 Parts A and B Secondary Endpoints

11.2.2.1 Efficacy per RECIST 1.1 Response Criteria
ORR: the proportion of subjects who have achieved a best overall response of CR or PR according to RECIST v1.1, as assessed by the investigator.
Best overall response: CR, PR, SD, progressive disease (PD), or not evaluable (NE).
Time to response (TTR): Time between the date of CTX130 infusion until first radiographically documented response (PR/CR).
Duration of response (DoR): Time between first objective response of PR/CR and date of disease progression or death due to any cause. This will be reported only for subjects who have had PR/CR events.
Progression-free survival (PFS): The difference between the date of CTX130 infusion and the date of disease progression or death due to any cause. Subjects who have not progressed and are still on study at the data cutoff date will be censored at their last RECIST assessment date.
Overall survival (OS): Time between the date of CTX130 infusion and death due to any cause. Subjects who are alive at the data cutoff date will be censored at the last date the subject was known alive.

11.2.2.2 Safety

The incidence and severity of AEs and clinically significant laboratory abnormalities are summarized and reported according to CTCAE version 5.0, except for CRS, which are graded according to Lee criteria (Lee et al., (2014) Blood 124, 188-195), neurotoxicity, which are graded according to ICANS (Lee et al., (2018) Biol Blood Marrow Transplant 25(4):625-638) and CTCAE v5.0, and GvHD, which are graded according to MAGIC criteria (Harris et al., (2016) Biol Blood Marrow Transplant, 22, 4-10).

11.2.2.3 Pharmacokinetics

The levels of CTX130 in blood and other tissues over time are assessed using a PCR assay that measures copies of CAR construct per μg DNA. Complementary analyses using flow cytometry to confirm the presence of CAR protein on the cellular surface may also be performed.

Such analyses may be used to confirm the presence of CTX130 in blood and to further characterize other cellular immunophenotypes.

11.2.3 Parts A and B Exploratory Endpoints
Levels of CTX130 in tissues. The expansion and persistence of CTX130 in tumor biopsy or CSF may be evaluated in any of these samples collected as per protocol-specific sampling.
Incidence of anti-CTX130 antibodies.
Immunoprofiling of lymphocyte populations.
Cytokine profile following administration of CTX130 product.
Impact of anti-cytokine therapy on effectiveness treating CRS, CTX130 proliferation, and the clinical response.
Incidence and type of subsequent (post CTX130) anti-cancer therapy.
Time to CR: Time between the date of the CTX130 infusion until documented CR.
Time to disease progression, defined as time between the date of CTX130 infusion until first evidence of disease progression.
First or second subsequent therapy-free survival: between date of the CTX130 infusion and date of first subsequent therapy or death due to any cause, or PFS.
Change from baseline in PROs, as measured by EORTC QLQ-C30, EQ-5D-5L, FKSI-19, and FACT-G questionnaires
Change from baseline in cognitive outcomes, as assessed by ICE
Other genomic, protein, metabolic, or pharmacodynamic endpoints.

### 11.3 Analysis Sets

The following analysis sets will be evaluated and used for presentation of the data:

### Part A (Dose Escalation)

The DLT-evaluable set will include all subjects who receive CTX130 and either have completed the DLT evaluation period following the initial infusion or have discontinued earlier after experiencing a DLT.

### Part A+Part B

Safety analysis set (SAS): All subjects who were enrolled and received at least 1 dose of study treatment. Subjects will be classified according to the treatment received, where treatment received is defined as the assigned dose level/schedule if it was received at least once, or the first dose level/schedule received if assigned treatment was never received. The SAS will be the primary set for the analysis of safety data.
Full analysis set (FAS): All subjects who were enrolled and received CTX130 infusion and have at least 1 baseline and 1 post baseline scan assessment. The FAS will be the primary analysis set for clinical activity assessment.

### 11.4 Sample Size and Power Consideration

Part A (dose escalation) sample size is approximately 6 to 18 evaluable subjects, depending on the number of dose levels evaluated and the occurrence of DLTs.

Part B (cohort expansion) will be a single-arm study conducted using an optimal Simon 2-stage design. In the first stage, at least 23 subjects will be enrolled and treated with CTX130. If ≥5 subjects achieve an objective response (CR or PR), it may be decided to expand the study to include an additional 48 treated subjects (71 total) in the second stage; otherwise, the enrollment will be paused. A sample size of 71 subjects will have 80% power (α=0.05, 2-sided test) to reject the null hypothesis that the ORR equals the historical response rate of 15% (Barata et al., 2018; Nadal et al., 2016; Powles et al., 2018), assuming the true ORR is 30%.

### 11.5 Statistical Analyses

Part A

Dose-limiting toxicities will be listed and their incidence summarized by Medical Dictionary for Regulatory Activities (MedDRA) primary System Organ Class (SOC) and/or Preferred Term (PT), worst grade based on CTCAE v5.0, type of AE, and dose level. The DLT-evaluable set will be the primary analysis set for evaluating DLTs in Part A.

Part B

The primary endpoint of ORR will be evaluated for subjects who have receive CTX130 at the RPBD in both Parts A and B. The FAS will be the primary analysis set for efficacy. Objective response rate will be summarized, and 95% confidence intervals (CIs) will be calculated.

Sensitivity analyses of ORR based on investigator review of disease assessments will also be performed.

General Efficacy Analysis

Time-to-event endpoints will be analyzed using Kaplan-Meier methods where appropriate. Estimates of the median and other quantiles (including 25th percentile and 75th percentile) based on the Kaplan-Meier method will be calculated and the associated 95% CIs will be provided. The survival rate at specific time points, based on the Kaplan-Meier method, will be produced. The time-to-event endpoints to be analyzed include:
Duration of response: Among responders only, DoR will be calculated as the date of the first occurrence of response to the date of documented disease progression or death, whichever occurs first. Subjects without disease progression or death will be censored at the last evaluable response assessment date.
Progression-free survival: Defined as duration from first date of study treatment until documented objective tumor progression or death. Subjects without disease progression or death will be censored at the last evaluable response assessment date.
Overall survival: Defined as the time between date of CTX130 infusion and death due to any cause. Subjects who are alive at the data cutoff date will be censored at the last date the subject was known alive.

General Safety Analysis

The SAS will be used for all listings and summaries of safety data. Safety data will be summarized by dose level.

Adverse Events

AEs will be graded according to CTCAE v5.0, except for CRS (ASTCT criteria), neurotoxicity (ICANS and CTCAE v5.0), and GvHD (MAGIC criteria). The incidence of treatment-emergent adverse events (TEAEs) will be summarized according to MedDRA by SOC and/or PT, severity (based on CTCAE v5.0), and relation to study treatment. Summaries of all TEAEs will be produced.

All AEs, regardless of start and end time, will be listed, and a flag indicating TEAE or not will be presented in the listing.

Laboratory Abnormalities
For laboratory tests covered by the CTCAE v5.0, laboratory data will be graded accordingly. For laboratory tests covered by CTCAE, Grade 0 will be assigned for all non-missing values not graded as 1 or higher.
The following summaries will be generated separately for hematology and chemistry laboratory tests:
   Descriptive statistics for the actual values (and/or change from baseline) or frequencies of clinical laboratory parameters over time
   Tables of the worst on-treatment CTCAE grades
   Listing of all laboratory data with values flagged to show the corresponding CTCAE grades and the classifications relative to the laboratory normal ranges

In addition to the above-mentioned tables and listings, graphical displays of key safety parameters, such as scatter plots of actual or change in laboratory tests over time or box plots may be specified in the SAP.

### 11.5 Interim Analyses

11.5.1 Efficacy Interim Analysis

One interim analysis for futility is performed and reviewed by the DSMB. The interim analysis occurs no later than when 22 subjects have been treated and have 3 months of evaluable response data. If the true response rate to CTX130 is not different from standard of care, the likelihood of stopping for futility at this analysis is 70%.

11.6.3 Biomarker Analysis

Incidence of anti-CTX130 antibodies, levels of CTX130 CAR+ T cells in blood, and levels of cytokines in serum are summarized.

Tumor, blood, possibly bone marrow and aspirate (only in subjects with treatment-emergent HLH), and possibly CSF samples (only in subjects with treatment-emergent neurotoxicity) will be collected to identify genomic, metabolic, and/or proteomic biomarkers that may be indicative of clinical response, resistance, safety, disease, pharmacodynamic activity, or the mechanism of action of CTX130.

Analysis of CTX130 Levels (Pharmacokinetic Analysis)

Analysis of levels of transduced CD70− directed CAR⁺ T cells will be performed on blood samples collected according to the schedule described in Table 21 and Table 22. In subjects experiencing signs or symptoms of CRS, additional blood samples should be drawn every 48 hours between scheduled collections. The time course of the expansion and persistence of CTX130 in blood will be described using a polymerase chain reaction (PCR) assay that measures copies of CAR construct. Complementary analyses using more sensitive genomic technology or flow cytometry to confirm the presence of CAR protein on the cellular surface may also be performed.

Samples for analysis of CTX130 levels should be sent to a central laboratory from blood, CSF (only in subject with treatment-emergent neurotoxicity), bone marrow (only in subjects with treatment-emergent HLH) or tumor biopsy performed following CTX130 infusion. The expansion and persistence of CTX130 in blood, CSF, bone marrow or tumor tissue may be evaluated in any of these samples collected as per protocol-specified sampling.

Cytokines

Cytokines including, but not limited to, CRP, IL-1β, sIL-1Rα, IL-2, sIL-2Rα, IL-4, IL-6, IL-8, IL-10, IL-12p70, IL-13, IL-15, IL-17a, interferon γ, tumor necrosis factor α, and GM-CSF, will be analyzed in a central laboratory. Correlational analysis performed in multiple prior CAR T cell clinical studies have identified these cytokines, and others, as potential predictive markers for severe CRS, as summarized in a recent review (Want et al, 2018). Blood for cytokines will be collected at specified times as described in Table 21 and Table 22. In subjects experiencing signs or symptoms of CRS, initial sample collection to occur at onset of symptoms, and additional samples should be drawn every 12 hours (±5 hours) until resolution.

Anti-CTX130 Antibody

The CAR construct is composed of humanized scFv. Blood is collected throughout the study to assess for potential immunogenicity following disclosures provided in this study.

Exploratory Research Biomarkers

Exploratory research may be conducted to identify molecular (genomic, metabolic, and/or proteomic) biomarkers and immunophenotypes that may be indicative or predictive of clinical response, resistance, safety, disease, pharmacodynamic activity, and/or the mechanism of action of treatment. Samples will be collected per Table 21 and Table 22. Refer to the Laboratory Manual for instructions on collection of blood, tumor, bone marrow, and CSF samples to support exploratory research.

Investigation of additional biomarkers may include assessment of blood cells and products, tumor tissue, and other subject-derived tissue. These assessments may evaluate DNA, RNA, proteins, and other biologic molecules derived from those tissues. Such evaluations inform understanding of factors related to patient disease, response to CTX130, and the mechanism of action of CTX130.

### Results

To date, all subjects that participated in this study have completed Stage 1 (eligibility screening) within 14 days. After having met the eligibility criteria, three subjects started lymphodepleting therapy within 24 hours of completing Stage 1. All eligible subjects have completed the screening period (stage 1) and received LD chemotherapy in less than 8 days, with two subject completing screening and starting an LD chemo dose within 72 hrs. All subjects receiving LD chemotherapy have progressed to receiving the DL1 dose of CTX130 within 2-3 days following completion of the LD chemotherapy.

None of the treated subjects in this study exhibited any DLTs so far. Similarly, no DTLs were observed in a parallel study using CTX130 to treat subjects with a T or B cell malignancy. See, e.g., U.S. Patent Application No. 62/934,945 filed Nov. 13, 2019 and U.S. Patent Application No. 63/034,510 filed Jun. 4, 2020. Further, the allogeneic CAR-T cell therapy exhibited desired pharmacokinetic features in the treated human subjects, including CAR-T cell expansion and persistence after infusion. Significant CAR T cell distribution, expansion and persistence has been observed as early as DL1. Up to 87-fold expansion of CTX130 in peripheral blood over T₀ has been observed in the one RCC subject evaluated to date and persistence of CTX130 cells can be detected in DL1 subjects at least 28 days following infusion. Similar patterns of CAR T cell distribution, expansion and persistence are observed in the corresponding T or B cell malignancy study, where 20-fold expansion of CTX130 has been observed and CTX130 cells have been detected up to 14 days post-infusion.

The eligible subjects in this study have clear cell RCC, some with minority fractions of sarcoid differentiation. Results obtained from the first two RCC subjects are summarized below.
The first subject receiving the DL1 dose experienced RCC stabilization of their tumor lesions without any new lesions or progression of exciting lesions per the CT scan at 42 days following CTX130 infusion. In addition, a lytic bone metastasis showed clear sings of recalcification in the same CT scan. The subject remained in stable disease at 12 weeks.
The second subject receiving the DL1 dose experienced at least a partial response at 42 days according to RECIST 1.1 with a drastic reduction of a subpleural target lesion and three non-target lesions in the thorax.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same FAShion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method for treating renal cell carcinoma (RCC), the method comprising:
(i) subjecting a human patient having RCC to a first lymphodepletion treatment;
(ii) administering to the human patient a first dose of a population of genetically engineered T cells after step (i), wherein the population of genetically engineered T cells comprises T cells expressing a chimeric antigen receptor (CAR) that binds CD70, a disrupted TRAC gene, a disrupted □2M gene, and a disrupted CD70 gene, and wherein a nucleotide sequence encoding the CAR is inserted into the disrupted TRAC gene.

2. The method of claim 1, wherein the first lymphodepletion treatment in step (i) comprises co-administering to the human patient fludarabine at 30 mg/m² and cyclophosphamide at 500 mg/m² intravenously per day for three days.

3. The method of claim 1, wherein prior to step (i), the human patient does not show one or more of the following features:
(a) significant worsening of clinical status,
(b) requirement for supplemental oxygen to maintain a saturation level of greater than 90%,
(c) uncontrolled cardiac arrhythmia,
(d) hypotension requiring vasopressor support,
(e) active infection, and
(f) Grade ≥2 acute neurological toxicity.

4. The method of claim 1, wherein step (i) is performed about 2-7 days prior to step (ii).

5. The method of claim 1, wherein step (ii) is performed by administering the population of genetically engineered T cells to the human patient intravenously at the first dose, which is about 1×10⁶ CAR⁺ cells to about 1×10⁹ CAR⁺ cells, optionally about 3×10⁷ to about 9×10⁸ CAR⁺ cells.

6. The method of claim 1, wherein prior to step (ii) and after step (i), the human patient does not show one or more of the following features:
(a) active uncontrolled infection,
(b) worsening of clinical status compared to the clinical status prior to step (i), and
(c) Grade ≥2 acute neurological toxicity.

7. The method of claim 1, further comprising (iii) monitoring the human patient for development of acute toxicity after step (ii).

8. The method of claim 7, wherein acute toxicity comprises cytokine release syndrome (CRS), neurotoxicity, tumor lysis syndrome, GvHD, on target off-tumor toxicity, and uncontrolled T cell proliferation, optionally wherein the neurotoxicity is immune effector cell-associated neurotoxicity (ICANS), and optionally wherein the on target off-tumor toxicity comprises activity of the population of genetically engineered T cells against activated T lymphocytes, B lymphocytes, dendritic cells, osteoblasts and/or renal tubular-like epithelium.

9. The method of claim 1, further comprising (iv) subjecting the human patient to a second lymphodepletion treatment, and (v) administering to the human patient a second dose of the population of genetically engineered T cells, wherein optionally the second dose is administered to the human patient about 8 weeks to about 2 years, optionally about 8-10 weeks or about 14-18 weeks, after the first dose.

10. The method of claim 9, further comprising (vi) subjecting the human patient to a third lymphodepletion treatment, and (vii) administering to the human patient a third dose of the population of genetically engineered T cells, wherein optionally the third dose is about 8 weeks to about 2 years, optionally about 8-10 weeks or about 14-18 weeks, after the second dose.

11. The method of claim 9 wherein the human patient does not show one or more of the following after step (ii) and/or after step (v):
(a) dose-limiting toxicity (DLT),
(b) Grade ≥3 CRS that does not resolve to ≤Grade 2 within 72 hours following step (ii) and/or step (v),
(c) Grade >1 GvHD,
(d) Grade ≥3 ICANS,
(e) active infection,
(f) hemodynamically unstable, and
(g) organ dysfunction.

12. The method of claim 9, wherein the second lymphodepletion treatment in step (iv), the third lymphodepletion treatment in step (vi), or both comprise co-administering to the human patient fludarabine at 30 mg/m² and cyclophosphamide at 500 mg/m² intravenously per day for 1-3 days.

13. The method of claim 9, wherein step (v) is performed 2-7 days after step (iv) and/or wherein step (vii) is performed 2-7 days after step (vi).

14. The method of claim 9, wherein step (v) and/or step (vii) is performed by administering the population of genetically engineered T cells to the human patient intravenously at the second dose and/or the third dose, which is about 1×10⁶ CAR⁺ cells to about 1×10⁹ CAR⁺ cells.

15. The method of claim 14, wherein the second dose and/or the third dose is about 3×10⁷ to about 9×10⁸ CAR+ cells.

16. The method of claim 9, wherein the human patient achieved a partial response (PR) or complete response (CR) after step (ii) and step (v) if applicable, and subsequently progressed within 2 years.

17. The method of claim 9, wherein the human patient achieved PR or stable disease (SD) after step (ii) and step (v) if applicable.

18. The method of claim 9, wherein the human patient is confirmed to have CD70⁺ RCC at replace prior to step (v) and step (vii) if applicable.

19. The method of claim 9, wherein the human patient shows stable disease or disease progress.

20. The method of claim 1, wherein the first dose, the second dose, and/or the third dose of the population of genetically engineered T cells is 1×10⁶ CAR⁺ cells, 3×10⁷ CAR⁺ cells, 1×10⁸ CAR⁺ cells, or 1×10⁹ CAR⁺ cells, optionally wherein the first dose, the second dose, and/or the third dose of the population of genetically engineered T cells is 1.5×10⁸ CAR⁺ cells, 4.5×10⁸ CAR⁺ cells, 6×10⁸ CAR⁺ cells, 7.5×10⁸ CAR⁺ cells, or 9×10⁸ CAR⁺ cells.

21. The method of claim 9, wherein the first dose of the population of genetically engineered T cells is the same as the second and/or third dose of the population of genetically engineered T cells.

22. The method of claim 9, wherein the first dose of the population of genetically engineered T cells is lower than the second and/or third dose of the population of genetically engineered T cells.

23. The method of claim 1, wherein the human patient has unresectable or metastatic RCC.

24. The method of claim 1, wherein the human patient has relapsed or refractory RCC.

25. The method of claim 1, wherein the human patient has clear cell differentiation.

26. The method of claim 1, wherein the human patient has undergone a prior anti-cancer therapy.

27. The method of claim 26, wherein the prior anti-cancer therapy comprises a checkpoint inhibitor, a tyrosine kinase inhibitor, a vascular growth factor inhibitor, or a combination thereof.

28. The method of claim 1, wherein the human patient is subject to an anti-cytokine therapy.

29. The method of claim 1, wherein the human patient is subject to an additional anti-cancer therapy after treatment with the population of genetically engineered T cells.

30. The method of claim 1, wherein the human patient has one or more of the following features:
(a) Karnofsky performance status (KPS) ≥80%, and
(b) adequate organ function,
(c) free of treatment with prior anti-CD70 or adoptive T cell or NK cell therapy,
(d) free of contraindications to lymphodepletion therapy,
(e) free of central nervous system (CNS) manifestation of malignancy,
(f) free of prior central nervous system disorders,
(g) free of pleural effusion or ascites or pericardial infusion,
(h) free of unstable angina, arrhythmia, and/or myocardial infarction,
(i) free of diabetes mellitus,
(j) free of uncontrolled infections,
(k) free of immunodeficiency disorders or autoimmune disorders that require immunosuppressive therapy,
(l) free of liver vaccine or herbal medicines, and
(m) free of solid organ transplantation or bone marrow transplant.

31. The method of claim 1, wherein the human patient is monitored for at least 28 days for development of toxicity after each administration of the population of genetically engineered T cells.

32. The method of claim 31, wherein the human patient is subject to toxicity management if development of toxicity is observed.

33. The method of claim 1, wherein the human patient is an adult.

34. The method of claim 1, wherein the CAR that binds CD70 comprises an extracellular domain, a CD8 transmembrane domain, a 4-1BB co-stimulatory domain, and a CD3ζ cytoplasmic signaling domain, and wherein the extracellular domain is a single-chain antibody fragment (scFv) that binds CD70.

35. The method of claim 34, wherein the scFv comprises a heavy chain variable domain (V_{H}) comprising SEQ ID NO: 49, and a light chain variable domain (VL) comprising SEQ ID NO: 50.

36. The method of claim 35, wherein the scFv comprises SEQ ID NO: 48.

37. The method of claim 34, wherein the CAR comprises SEQ ID NO: 46.

38. The method of claim 1, wherein the disrupted TRAC gene is produced by a CRISPR/Cas9 gene editing system, which comprises a guide RNA comprising a spacer sequence of SEQ ID NO: 8 or 9.

39. The method of claim 38, wherein the disrupted TRAC gene has a deletion of the region targeted by the spacer sequence of SEQ ID NO: 8 or 9, or a portion thereof.

40. The method of claim 1, wherein the disrupted β2M gene is produced by a CRISPR/Cas9 gene editing system, which comprises a guide RNA comprising a spacer sequence of SEQ ID NO: 12 or 13.

41. The method of claim 1, wherein the disrupted CD70 gene is produced by a CRISPR/Cas9 gene editing system, which comprises a guide RNA comprising a spacer sequence of SEQ ID NO: 4 or 5.


SEQUENCE LISTING (69 sequences)

SEQ ID NO: 1 - protein, 1368 aa, Streptococcus pyogenes [sequence omitted]

SEQ ID NO: 2 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuuuggucccauuggucgcguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence; identical to SEQ ID NO: 2
  Synthetic

SEQ ID NO: 4 - RNA, 20 nt, Artificial Sequence
  Synthetic
  misc_feature (1)..(4): modified with 2′-O-methyl phosphorothioate
gcuuuggucccauuggucgc

SEQ ID NO: 5 - RNA, 20 nt, Artificial Sequence
  Synthetic
gcuuuggucccauuggucgc

SEQ ID NO: 6 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
agagcaacagugcuguggccguuuuagagcuagaaauagcaaguuaaaauaaggcuaguccguuaucaacuugaaaaaguggcaccgagucggugcuuuu

SEQ ID NO: 7 - RNA, 100 nt, Artificial Sequence; identical to SEQ ID NO: 6
  Synthetic

SEQ ID NO: 8 - RNA, 20 nt, Artificial Sequence
  Synthetic
  misc_feature (1)..(4): modified with 2′-O-methyl phosphorothioate
agagcaacagugcuguggcc

SEQ ID NO: 9 - RNA, 20 nt, Artificial Sequence
  Synthetic
agagcaacagugcuguggcc

SEQ ID NO: 10 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuacucucucuuucuggccguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence; identical to SEQ ID NO: 10
  Synthetic

SEQ ID NO: 12 - RNA, 20 nt, Artificial Sequence
  Synthetic
  misc_feature (1)..(4): modified with 2′-O-methyl phosphorothioate
gcuacucucucuuucuggcc

SEQ ID NO: 13 - RNA, 20 nt, Artificial Sequence
  Synthetic
gcuacucucucuuucuggcc

SEQ ID NO: 14 - DNA, 23 bp, Artificial Sequence
  Synthetic
gctttggtcccattggtcgcggg

SEQ ID NO: 15 - DNA, 20 bp, Artificial Sequence
  Synthetic
gctttggtcccattggtcgc

SEQ ID NO: 16 - DNA, 23 bp, Artificial Sequence
  Synthetic
agagcaacagtgctgtggcctgg

SEQ ID NO: 17 - DNA, 20 bp, Artificial Sequence
  Synthetic
agagcaacagtgctgtggcc

SEQ ID NO: 18 - DNA, 23 bp, Artificial Sequence
  Synthetic
gctactctctctttctggcctgg

SEQ ID NO: 19 - DNA, 20 bp, Artificial Sequence
  Synthetic
gctactctctctttctggcc

SEQ ID NO: 20 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 21 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 22 - RNA, 114 nt, Artificial Sequence
  Synthetic
  misc_feature (1)..(17): n is a, c, g, or u
  misc_feature (18)..(30): may be absent
  misc_feature (109)..(114): may be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 23 - DNA, 19 bp, Artificial Sequence
  Synthetic
aagagcaacaaatctgact

SEQ ID NO: 24 - DNA, 39 bp, Artificial Sequence
  Synthetic
aagagcaacagtgctgtgcctggagcaacaaatctgact

SEQ ID NO: 25 - DNA, 33 bp, Artificial Sequence
  Synthetic
aagagcaacagtgctggagcaacaaatctgact

SEQ ID NO: 26 - DNA, 34 bp, Artificial Sequence
  Synthetic
aagagcaacagtgcctggagcaacaaatctgact

SEQ ID NO: 27 - DNA, 19 bp, Artificial Sequence
  Synthetic
aagagcaacagtgctgact

SEQ ID NO: 28 - DNA, 41 bp, Artificial Sequence
  Synthetic
aagagcaacagtgctgtgggcctggagcaacaaatctgact

SEQ ID NO: 29 - DNA, 38 bp, Artificial Sequence
  Synthetic
aagagcaacagtgctggcctggagcaacaaatctgact

SEQ ID NO: 30 - DNA, 41 bp, Artificial Sequence
  Synthetic
aagagcaacagtgctgtgtgcctggagcaacaaatctgact

SEQ ID NO: 31 - DNA, 79 bp, Artificial Sequence
  Synthetic
cgtggccttagctgtgctcgcgctactctctctttctgcctggaggctatccagcgtgagtctctcctaccctcccgct

SEQ ID NO: 32 - DNA, 78 bp, Artificial Sequence
  Synthetic
cgtggccttagctgtgctcgcgctactctctctttcgcctggaggctatccagcgtgagtctctcctaccctcccgct

SEQ ID NO: 33 - DNA, 75 bp, Artificial Sequence
  Synthetic
cgtggccttagctgtgctcgcgctactctctctttctggaggctatccagcgtgagtctctcctaccctcccgct

SEQ ID NO: 34 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 35 - DNA, 55 bp, Artificial Sequence
  Synthetic
cgtggccttagctgtgctcgcgctatccagcgtgagtctctcctaccctcccgct

SEQ ID NO: 36 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 37 - DNA, 58 bp, Artificial Sequence
  Synthetic
cacaccacgaggcagatcaccaagcccgcgcaatgggaccaaagcagcccgcaggacg

SEQ ID NO: 38 - DNA, 61 bp, Artificial Sequence
  Synthetic
cacaccacgaggcagatcaccaagcccgcgaaccaatgggaccaaagcagcccgcaggacg

SEQ ID NO: 39 - DNA, 48 bp, Artificial Sequence
  Synthetic
cacaccacgaggcagatcaccaatgggaccaaagcagcccgcaggacg

SEQ ID NO: 40 - DNA, 59 bp, Artificial Sequence
  Synthetic
cacaccacgaggcagatcaccaagcccgcgccaatgggaccaaagcagcccgcaggacg

SEQ ID NO: 41 - DNA, 59 bp, Artificial Sequence
  Synthetic
cacaccacgaggcagatcaccaagcccgcaccaatgggaccaaagcagcccgcaggacg

SEQ ID NO: 42 - DNA, 35 bp, Artificial Sequence
  Synthetic
cacaccacgaggcagatcaccaagcccgcaggacg

SEQ ID NO: 43 - DNA, 4688 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 45 - DNA, 1527 bp, Artificial Sequence
  Synthetic
atggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataa

SEQ ID NO: 46 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 735 bp, Artificial Sequence
  Synthetic
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 48 - protein, 245 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 49 - protein, 118 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 50 - protein, 111 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 51 - DNA, 16 bp, Artificial Sequence
  Synthetic
ggggsggggsggggsg

SEQ ID NO: 52 - protein, 22 aa, Artificial Sequence
  Synthetic
MLLLVTSLLLCELPHPAFLLIP

SEQ ID NO: 53 - protein, 21 aa, Artificial Sequence
  Synthetic
MALPVTALLLPLALLLHAARP

SEQ ID NO: 54 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 55 - protein, 23 aa, Artificial Sequence
  Synthetic
IYIWAPLAGTCGVLLLSLVITLY

SEQ ID NO: 56 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 42 aa, Artificial Sequence
  Synthetic
KRGRKKLLYIFKQPFMRPVQTTQEEDGCSCRFPEEEEGGCEL

SEQ ID NO: 58 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 59 - protein, 40 aa, Artificial Sequence
  Synthetic
SKRSRLLHSDYMNMTPRRPGPTRKHYQPYAPPRDFAAYRS

SEQ ID NO: 60 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 61 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 63 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 64 - DNA, 49 bp, Artificial Sequence
  Synthetic
aataaaatcgctatccatcgaagatggatgtgtgttggttttttgtgtg

SEQ ID NO: 65 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 66 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcccgcaggacgcacccauaguuuuagagcuagaaauagcaaguuaaaauaaggcuaguccguuaucaacuugaaaaaguggcaccgagucggugcuuuu

SEQ ID NO: 67 - RNA, 100 nt, Artificial Sequence; identical to SEQ ID NO: 66
  Synthetic

SEQ ID NO: 68 - RNA, 20 nt, Artificial Sequence
  Synthetic
  misc_feature (1)..(4): modified with 2′-O-methyl phosphorothioate
gcccgcaggacgcacccaua

SEQ ID NO: 69 - RNA, 20 nt, Artificial Sequence
  Synthetic
gcccgcaggacgcacccaua